(19) United States
(12) Patent Application Publication
(10) Pub. No.: US 2025/0369955 A1
(43) Pub. Date: Dec. 4, 2025

(54) MOUSE MODELS OF CYTOKINE RELEASE SYNDROME

(71) Applicant: The Jackson Laboratory, Bar Harbor, ME (US)

(72) Inventor: James Keck, Granite Bay, CA (US)

(73) Assignee: The Jackson Laboratory, Bar Harbor, ME (US)

(21) Appl. No.: 18/876,747

(22) PCT Filed: Jun. 26, 2023
(86) PCT No.: PCT/US2023/026194

§ 371 Date: Dec. 19, 2024

Related U.S. Application Data
(60) Provisional application No. 63/355,941, filed on Jun. 27, 2022.

(51) Int. Cl. G01N 33/50; A01K 67/0271; A01K 67/0276; A01K 67/0278

(52) U.S. Cl. CPC G01N 33/5088; A01K 67/0271; A01K 67/0276; A01K 67/0278; A01K 2207/12; A01K 2207/15; A01K 2217/072; A01K 2217/075; A01K 2227/105; A01K 2267/0387; G01N 2333/5412; G01N 2333/5428; G01N 2333/57

(57) ABSTRACT

Provided herein are humanized mouse models generated using T cell-negative fractions or peripheral blood mononuclear cells obtained from T cell-negative fractions, and methods of using the mouse models to assess the efficacy and/or side effects of a therapeutic agent. Immune cell therapies require a large number of cells. Most commonly, the cells are collected using a process referred to as apheresis. Apheresis collection of the mononuclear cell (MNC) layer has been shown to be a safe and efficient method of collecting the large number of T cells.

### RELATED APPLICATION

This application claims the benefit under 35 U.S.C. § 119 (e) of U.S. provisional application No. 63/355,941, filed Jun. 27, 2022, which is incorporated by reference herein in its entirety.

### BACKGROUND

Adoptive cell therapy (ACT), such as chimeric antigen receptor (CAR) immune cell therapy (e.g., CAR T cell therapy or CAR-natural killer cell (CAR-NK) therapy) has become a revolutionary cancer treatment. It has proven to be an effective treatment for hematological malignancies and is currently being developed to treat solid tumor cancers. ACT utilizes gene transfer to reprogram immune cells expressing an engineered antigen receptor, which enables immune cells (e.g., T cells, B cells, and/or NK cells) to recognize and target (bind to) cell surface antigens specific to a diseased cell, such as a tumor cell, further eliminating diseased cells carrying the antigen. Currently, there are six Food and Drug Administration (FDA)-approved CAR T cell products, for example: three for the treatment of B-cell lymphoma, two for multiple myeloma and one for the treatment of advanced mantle cell lymphoma (MCL). Similarly, NK cells play a pivotal role as the body's first-line defense against virally infected and malignant cells.

### SUMMARY

Immune cell therapies require a large number of cells. Most commonly, the cells are collected using a process referred to as apheresis. Apheresis collection of the mononuclear cell (MNC) layer has been shown to be a safe and efficient method of collecting the large number of T cells, for example. Circulating mature lymphocytes can be found within the MNC layer; therefore, isolation of this layer provides the cells to begin manufacturing engineered T cells. Following the collection of the T cells, the “leftover components,” collectively referred to as a T cell-negative fraction, are often discarded as waste. The inventors have demonstrated, as described herein, that the T cell-negative fraction is not waste but rather is a rich source of immune cells, such as peripheral blood mononuclear cells (PBMCs), that may be used to humanize immunodeficient mouse models, which may in turn be used to test the efficacy and/or side-effects of an intended (or candidate) T cell therapy or other therapeutic modality.

This model is particularly useful, in some embodiments, for assessing immune cell therapies for patients who suffer from a late-stage (e.g., Stage 3/Stage4) cancer, for example, those who have already undergone one or more anti-cancer therapies. PBMCs obtained from late-stage cancer patients that have undergone treatment include a population of (treated) T cells that induce graft-versus-host disease (GVHD) when administered to immunodeficient mice; thus, they cannot be engrafted in the mice and cannot be used to humanize the mice. The inventors have solved this GVHD problem by using the T cell-negative fraction(s) obtained from patients, or the PBMCs obtained from the T cell-negative fraction(s), to humanize the mice. The T cell-negative fractions minimize the risk of GVHD in mice, thus can be used as a source of human immune cells to humanize immunodeficient mice, providing models with which to test immune cell therapies and other therapies for cancer patients, such as late-stage cancer patients.

The most common side effects of immune cell therapy (such as ACT, e.g., CAR T cell, CAR B cell, and/or CAR NK cell therapy), which can be assessed using the mouse models of the present disclosure, are cytokine release syndrome (CRS) and encephalopathy syndrome (neurotoxicity)—two major complications that can lead to significant morbidity and mortality. CRS is a cytokine-mediated systemic inflammatory response caused by multiple cytokines following in vivo immune cell (e.g., T cell, B cell, NK cell) activation and expansion. Immune cells, for example, those comprising an engineered antigen receptor, and diseased cells (e.g., tumor cells) can contribute to the induction of CRS by releasing cytokines. The main cytokines associated with pathogenesis of CRS include interleukin (IL) 6, IL10, interferon (IFN)-γ, monocyte chemoattractant protein 1 (MCP-1), and granulocyte-macrophage colony-stimulating factor (GM-CSF). Several other cytokines, including but not limited to tumor necrosis factor (TNF), IL1, IL2, IL2 receptor alpha (IL2Rα), and IL8 have also been implicated in CRS development. Although the mechanism of CRS is not well understood, several factors contributing to this toxicity include the structure of a chimeric antigen receptor, high tumor burden, higher engineered immune cell (e.g., T cell, B cell, NK cell) infusion dose, and other patient-specific factors, such as pre-existent state of inflammation and baseline endothelial activation.

Preclinical models of CRS, such as those provided herein, are useful for identifying agents effective for CRS treatment that do not interfere with the cytokine-mediated anti-tumor effects of engineered immune cells (e.g., engineered T cells, engineered B cells, and/or engineered NK cells). In addition, preclinical models of CRS are helpful for evaluating which engineered immune cells induce the least cytokine release and remain therapeutically effective.

It should be understood that the term “engineered immune cell” herein refers to any immune cell (e.g., T cell, B cell, or NK cell) that comprises and/or expresses an engineered antigen receptor, i.e., a non-naturally-occurring receptor that specifically binds to a cell surface antigen of interest or that comprises one or more other genomic modification(s) (e.g., mutation/substitution, insertion, deletion, or indel (insertion and deletion)). For example, a “CAR immune cell” such as a “CAR T cell”, “CAR B cell”, or “CAR NK cell” is considered an “engineered immune cell.” Other examples of engineered immune cells include T cells with an engineered T cell receptor (TCR), engineered (e.g., genome-edited) tumor infiltrating lymphocytes (eTIL) and engineered regulatory T cells (eTregs).

Some aspects relate to a method comprising: administering a T cell-negative fraction, or cells from the T cell-negative fraction, to a mouse, optionally an immunodeficient mouse, wherein the T cell-negative fraction is from a cancer patient.

In some embodiments, the method further comprises administering human cancer cells to the mouse. For example, the human cancer cells may be from the cancer patient.

In some embodiments, the method further comprises administering a therapeutic agent to the mouse.

In some embodiments, the method further comprises assaying the mouse for one or more human cytokines prior to the onset of graft-versus-host disease (GVHD) in the mouse. GVHD is condition that occurs when donated stem cells or bone marrow (the graft) see the healthy tissues in the host (e.g., mouse) as foreign and attack them. GVHD can cause damage to the host's tissues and organs, especially the skin, liver, intestines, eyes, mouth, hair, nails, joints, muscles, lungs, kidneys, and genitals. The signs and symptoms may be severe and life threatening. GVHD in humans can occur within the first few months after transplant (acute) or much later (chronic). In some embodiments, GVHD in a mouse can occur about 4 weeks to about 10 weeks (e.g., about 4, 5, 6, 7, 8, 9, or about 10 weeks) following administration of a T cell-negative fraction or cells from the T cell-negative fraction.

In some embodiments, about 1×10\hat{ }6 to about 1×10\hat{ }8 cells, optionally about 0.5×10\hat{ }7 to about 3×10\hat{ }7 cells, from the T cell-negative fraction are administered to the mouse. In some embodiments, about 1.5×10\hat{ }7 cells from the T cell-negative fraction are administered to the mouse.

In some embodiments, the T cell-negative fraction comprises about 1×10\hat{ }6 to about 1×10\hat{ }8 cells, optionally about 0.5×10\hat{ }7 to about 3×10\hat{ }7 cells. In some embodiments, the T cell-negative fraction comprises about 1.5×10\hat{ }7 cells.

In some embodiments, the cells are human peripheral blood mononuclear cells (PBMCs).

In some embodiments, the cancer patient is a Stage 3 or Stage 4 cancer patient, optionally wherein the cancer patient has undergone one or more anti-cancer therapies.

In some embodiments, the cancer patient is younger than 18 years old (e.g., younger than 15 years old, younger than 10 years old, or younger than 5 years old).

In some embodiments, the therapeutic agent is administered within 10 days (e.g., within 8 days, or about 3 to 10 days, about 3 to 8) of administering the T cell-negative fraction, or the cells from the T cell-negative fraction, to the mouse.

In some embodiments, the therapeutic agent is selected from an engineered immune cell, a recombinant protein, a nucleic acid, and a small molecule drug. In some embodiments, the therapeutic agent is an engineered immune cell. For example, the engineered immune cell may be a T cell, an NK cell, or a B cell. In some embodiments, the engineered immune cell is a T cell, for example, a regulatory T cell (Treg) or a tumor infiltrating lymphocyte (TIL).

In some embodiments, the engineered immune cell comprises a chimeric antigen receptor (CAR). In other embodiments, the engineered immune cell comprises a T cell receptor.

In some embodiments, the recombinant protein is an antibody, for example, an antibody fragment.

In some embodiments, the nucleic acid is an antisense oligonucleotide (ASO), a short interfering RNA (siRNA), a messenger RNA (mRNA), or a viral vector, for example, an adeno-viral vector (AAV).

In some embodiments, the assaying is within 10 days (e.g., within 8 days, or about 3 to 10 days, about 3 to 8) of administering the therapeutic agent to the mouse.

In some embodiments, the one or more human cytokines is selected from interleukin-6 (IL-6), IL-10, and interferon (IFN)-γp (e.g., from sera from the mouse) In some embodiments, IL-6, IL-10 and IFN-γ is measured.

In some embodiments, the mouse has undergone a myeloablative treatment, for example, gamma irradiation or chemical treatment.

In some embodiments, the mouse is an immunodeficient mouse. For example, the mouse may have an NSG®, BRG or NCG genetic background.

In some embodiments, the mouse has a non-obese diabetic (NOD) genetic background.

In some embodiments, the mouse comprises a null mutation in a Prkdc gene, for example, comprises a Prkdc^{scid} allele. In some embodiments, the mouse comprises a null mutation in an Il2rg gene, for example, comprises a IL2rg^{tmlwJl} allele.

In some embodiments, the mouse comprises a null H2-Abl gene, for example, comprises a H2-Abl^{emlMvw} allele. In some embodiments, the mouse comprises a null MHC Class I H2-Kl gene, for example, comprises a H2-Kl^{tmlBpe} allele. In some embodiments, the mouse comprises a null MHC Class I H2-Dl gene, for example, comprises a H2-Dl^{tmlBpe} allele.

In some embodiments, the mouse comprises a transgene encoding human interleukin-3 (IL-3), a transgene encoding human granulocyte/macrophage-colony stimulating factor 2 (GM-CSF), a transgene encoding human stem cell factor (SCF), and optionally further comprises a transgene encoding human macrophage colony-stimulating factor 1 (CSF1) and/or a transgene encoding human IL-15.

In some embodiments, the mouse comprises a nucleic acid encoding human FLT3L protein and/or a null mutation in a mouse Flt3 gene, optionally a Flt3^{em}2Mvw allele.

In some embodiments, the administering is intravenous, for example, by tail vein injection. In some embodiments, the administering is intraperitoneal.

In some embodiments, the T cell-negative fraction is a CD3⁻T cell-negative fraction, for example, a CD3⁻CD4⁻T cell-negative fraction and/or CD3⁻CD8⁻T cell-negative fraction.

### BRIEF DESCRIPTION OF THE DRAWINGS

FIG. 1A depicts mean body weight change in three mouse strains (DKO, SGM3xIL15, and FLT3L) after engraftment of human patient CD3-negative fraction (1.5×10⁷ cells/mouse). There were 2 mice per group and data are presented as mean±SEM.

FIG. 1B depicts the change in body weight over time in different strains bodyweight change of individual FLT3L mice after engraftment of human CD3-negative fraction 1.5×10⁷ cells/mouse. Each line represents one mouse.

FIG. 1C depicts the change in body weight over time in different strains bodyweight change of individual SGM3xIL15 mice after engraftment of human CD3-negative fraction 1.5×10⁷ cells/mouse. Each line represents one mouse.

FIG. 1D depicts the change in body weight over time in different strains bodyweight change of individual DKO mice after engraftment of human CD3-negative fraction 1.5×10⁷ cells/mouse. Each line represents one mouse.

FIG. 2A shows the absolute counts of human CD45+ cells per microliter of blood samples collected 5, 7, 9 days after the injection of human negative fraction cells (Day 5, Day 7, Day 9). Data are presented as mean±SEM.

FIG. 2B depicts CD3+, CD19+, CD14+/16+, CD56+ cell populations as a percentage of human CD45+ cells (gated on human CD45+cells) for the blood samples collected from FLT3L mice. Data are presented as mean±SEM.

FIG. 2C depicts CD3+, CD19+, CD14+/16+, CD56+ cell populations as a percentage of human CD45+ cells (gated on human CD45+ cells) for the blood samples collected from SGM3xIL 15 mice. Data are presented as mean±SEM.

FIG. 2D depicts CD3+, CD19+, CD14+/16+, CD56+ cell populations as a percentage of human CD45+ cells (gated on human CD45+ cells) for the blood samples collected from DKO mice. Data are presented as mean±SEM.

FIG. 3 depicts the body weight loss as a result of toxicity induced by allogeneic chimeric antigen receptor T (CAR T) treatment. After 6 days, the engraftment of human negative fraction cells, each mouse was treated with CAR T cells (5×10⁶ cells/mouse). The number of mice for each group was two for FLT3L strain, one for SGM3xIL15 strain and one for DKO strain. Each line represents one mouse. One FLT3L mouse and one SGM3xIL15 mouse showed significant body weight loss 3 days after the CAR T treatment.

FIG. 4 depicts clinical scores for cytokine release syndrome (CRS) symptoms after CAR T treatment. CRS 0=normal activity; CRS 1=normal activity, hunched +/− piloerection; CRS 2=hunched with reduced activity, but will still move around the cage without continued stimulation; CRS 3=not moving unless being stimulated; will move when touched, but will stop moving soon after the hand is removed; CRS 4=moribund (non-responsive to touch). Each line represents one mouse.

FIG. 5A depicts the human CD45+ cell counts per microliter in FLT3L, SGM3xIL15, and DKO mice from the blood collected on the day of CAR T treatment (Pre-CAR T) and 6 days after the CAR T treatment (6D post CAR T).

FIG. 5B depicts CD3+, CD19+, CD14+/16+, CD56+ cell populations as a percentage of human CD45+ cells (gated on human CD45+ cells) for the blood samples collected from CAR T-treated FLT3L mice.

FIG. 5C depicts CD3+, CD19+, CD14+/16+, CD56+ cell populations as a percentage of human CD45+ cells (gated on human CD45+ cells) for the blood samples collected from a CAR T-treated SGM3xIL 15 mouse.

FIG. 5D depicts CD3+, CD19+, CD14+/16+, CD56+ cell populations as a percentage of human CD45+ cells (gated on human CD45+ cells) for the blood samples collected from a CAR T-treated DKO mouse.

FIG. 6A shows cytokine concentrations measured on the day of treatment (Pre-CAR T) and 2 and 6 days after CAR T treatment in FLT3L mice that were engrafted with human CD3-negative fraction cells.

FIG. 6B shows cytokine concentrations measured on the day of treatment (Pre-CAR T) and 2 and 6 days after CAR T treatment in a SGM3xIL15 mouse that was engrafted with human CD3-negative fraction cells.

FIG. 6C shows cytokine concentrations measured on the day of treatment (Pre-CAR T) and 2 and 6 days after CAR T treatment in a DKO mouse that was engrafted with human CD3-negative fraction cells.

FIG. 7A shows fold changes of cytokines measured on 2- and 6-days post CAR T treatment (light gray and dark gray, respectively) relative to the cytokines measured on Pre-CART in FLT3L mice.

FIG. 7B shows fold changes of cytokines measured on 2- and 6-days post CAR T treatment (light gray and dark gray, respectively) relative to the cytokines measured on Pre-CART in a SGM3xIL 15 mouse.

FIG. 7C shows fold changes of cytokines measured on 2- and 6-days post CAR T treatment (light gray and dark gray, respectively) relative to the cytokines measured on Pre-CART in a DKO mouse.

FIG. 8A shows body weight change over time in SGM3xIL15 mice after engraftment of human patient CD3-negative fraction, the raw fraction and the processed PBMCs (1.5×10⁷ cells/mouse).

FIG. 8B shows body weight change over time in SGM3xIL15 mice after engraftment of human patient CD3-negative fraction, the raw negative fraction or processed PBMCs from the negative fraction (1.5×10⁷ cells/mouse) and then administration of CAR T therapy (arrow).

FIG. 9 depicts human cell engraftment (number of live cells) in SGM3xIL15 mice six days after engraftment with 1.5×10⁷cells/mouse of the raw negative fraction or processed PBMCs from the negative fraction.

FIG. 10 depicts allogeneic CAR T efficacy (decrease of CD19+ cells) in SGM3xIL15 mice humanized with processed PBMCs from the negative fraction (“pre”) and six days after administration of allogeneic CAR T therapy (“6D”).

FIG. 11 depicts cytokine concentrations measured 2 and 6 days after allogeneic CAR T treatment in a SGM3xIL15 mice that were engrafted with human CD3-negative fraction cells (raw or processed PBMCs).

FIG. 12 depicts allogeneic CAR T cell expansion in SMG3xIL15 mice humanized with 1.5×10⁷ cells/mouse of the raw negative fraction or processed PBMCs from the negative fraction and administration of allogeneic CAR T treatment. Data is from before administration of CAR T treatment (“pre”) and six days later (“6D”).

### DETAILED DESCRIPTION

Engineered immune cell therapies use gene transfer to reprogram immune cells (e.g., T cells, B cells, NK cells) so that they express at least one engineered antigen receptor (e.g., CAR or TCR), enabling the resulting immune cells to recognize and target cell surface antigens specific to a particular disease (e.g., cancer) or cell type. For example, CAR T cells eliminate malignant cells after recognizing and binding to an antigen expressed on the surface of the malignant cells. In this way, engineered immune cell (e.g., T cell, B cell, or NK cell) therapy is used to treat hematological malignancies and is currently being developed to treat solid tumor cancers. Similarly, engineered immune cells can be used to target (e.g., bind to) cell surface antigens specific to other diseased cells, for example, those associated with cardiovascular disease, metabolic disease, or other pathological states.

Engineered immune cell therapies, such as engineered T cell therapies, have several known side effects, such as cytokine release syndrome (CRS) and T cell-related encephalopathy syndrome (neurotoxicity). Either or both complications can lead to significant morbidity and mortality.

An additional or alternative cancer immunotherapy includes the use of CARs to reprogram natural killer cells. CAR natural killer cell (CAR NK) therapy can be an off-the-shelf (e.g., universal) therapy, as NK cells do not require strict human leukocyte antigen (HLA) matching or carry the risk of graft-versus-host disease. CAR NK therapy is developing, as primary NK cell isolation, expansion, and transduction are still being refined.

Other immune cells, such as B cells, dendritic cells, monocytes/macrophages, and neutrophils, may also be reprogramed to express at least one engineered antigen receptor (e.g., CAR).

As described herein, T cell-negative fractions (e.g., apheresis products with the T cell populations removed) or PBMCs obtained from T cell-negative fractions from late-stage cancer patients were used to effectively humanize mice. A T cell-negative fraction includes human immune cells (e.g., PBMCs, monocytes and NK cells) that are capable of releasing human cytokines. In this way, the humanized mouse models described herein more precisely represent in vivo CRS induction and enable a more accurate assessment of human cytokine release, for example, in late-stage cancer patients. Thus, the mouse models described herein may be used to assess whether a particular therapeutic agent, such as an engineered immune cell therapy, is likely to be associated with CRS or other side effect(s). The mouse models describe herein may also be used to assess the effectiveness of therapeutic agents (e.g., immune cells) effective for treating certain diseases (e.g., cancers) without inducing CRS, or to identify candidate agents for treating CRS without interfering with the therapeutic efficacy of the therapeutic agent.

### T Cell-Negative Fractions

In some embodiments, immunodeficient mice are engrafted with a T cell-negative fraction of human immune cells, thereby humanizing the mice. As used herein, “T cell-negative fraction” refers to a biological sample that has been obtained from a subject (e.g., an apheresis sample) and processed to remove T cells (e.g., CD4+ helper T cells and/or CD8+ killer T cells), for example, using CD3/CD28 antibody selection. In some embodiments, a T cell-negative fraction lacks T cells entirely. In other embodiments, less than 5%, less than 4%, less than 3%, less than 2%, less than 1%, less than 0.5%, or less than 0.1% of the cells of a T cell-negative fraction comprises T cells. In some embodiments, the T cell-negative fraction is obtained from a blood (e.g., serum or plasma) sample, for example, drawn to obtain T cells for a T cell therapy.

In other embodiments, human PBMCs are obtained from (e.g., isolated/purified from) a T cell-negative fraction, and then administered to an immunodeficient mouse. Thus, in some embodiments, the immunodeficient mice are engrafted with human PBMCs obtained from a T cell-negative fraction, thereby humanizing the mice.

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of PBMCs: lymphocytes and monocytes. The majority (˜70-90%) of an enriched human PBMC sample is composed of lymphocytes (white blood cells), which include CD4+ helper T cells, CD8+ killer T cells, B cells, and Natural Killer (NK) cells. Monocytes make up a smaller portion (˜10-30%) of the enriched human PBMC sample. Monocytes, when stimulated, can differentiate into macrophages or dendritic cells. As described herein, T cell-negative fraction or PBMCs obtained from a T cell-negative fraction may be used for engraftment.

The T cell-negative fraction may be isolated from whole blood samples, and in some embodiments, further processed to obtain the PBMCs, for example, using a Ficoll gradient. T cell-negative fractions from a subject (e.g., a human subject) with a current or previous diagnosis of cancer or an autoimmune disease may be used. In some embodiments, the subject is a pediatric subject, e.g., a pediatric subject is younger than 18 years old (e.g., 1, 2, 3, 4, 5, 6, 7, 8, 9, 10, 11, 12, 13, 14, 15, 16, or 17 years old). In some embodiments, a pediatric is younger than a year old. In some embodiments, a subject is older than 18 years old. In some embodiments, a subject (e.g., a pediatric subject) has a current diagnosis of Stage 3 cancer (e.g., locally advanced cancer). In some embodiments, a subject (e.g., a pediatric subject) has a current diagnosis of Stage 4 cancer (e.g., advanced cancer, metastatic cancer). In some embodiments, the subject is a pediatric subject having a current diagnosis of Stage 3 or Stage 4 cancer.

### Therapeutic Modalities

Therapeutic modalities are the different approaches and strategies used in the treatment of various diseases and health conditions in a subject. Herein, the terms “subject,” “patient,” and “individual” are used interchangeably. In some embodiments, a subject is a human subject. Other animal subjects are also contemplated herein. Some of the most common therapeutic modalities include pharmacotherapy, which involves the use of drugs to treat diseases and manage symptoms. Other therapeutic modalities include gene therapy and immunotherapy, which use genetic manipulation and the immune system, respectively, to treat diseases such as cancer and genetic disorders. There are many therapeutic modalities available, and the choice of treatment depends on the patient's condition, medical history, and the expertise of the healthcare provider.

The therapeutic modality, in some embodiments, is a targeted therapeutic. Targeted therapy is a type of treatment, for example, cancer, anti-inflammatory, or infection disease treatment, that uses drugs or other substances to identify and attack cells more precisely than standard therapies. Unlike chemotherapy, for example, which can affect healthy cells as well as cancer cells, targeted therapy is designed to interfere with specific molecules (e.g., cancer antigens) or pathways involved in cancer cell growth and survival. Targeted therapy is based on the principle that diseased cells often have certain genetic or molecular abnormalities that distinguish them from normal cells. By targeting these specific abnormalities, targeted therapies can be more effective and less toxic than traditional therapies, such as chemotherapy. Non-limiting examples of targeted therapies include drugs that block the activity of specific enzymes or growth factor receptors, as well as immunotherapies that stimulate the immune system to recognize and attack cancer cells.

In some embodiments, the mouse models provided herein are used to assess the on-target (e.g., on tumor) and off-target (e.g., off-tumor) effects of a therapeutic modality. Non-limiting examples of therapeutic modalities that may be used as provided herein include antibodies, small molecule drugs, gene therapies, cell therapies, vaccines, hormones, enzyme replacement therapies, and nucleic acid-based therapies.

Antibodies are proteins produced by the immune system that can specifically recognize and bind to foreign substances, such as viruses and bacteria, and help neutralize or eliminate them from the body. Antibodies can also be designed and produced in the laboratory and used as therapeutics to target specific proteins or cells in the body. A therapeutic antibody used herein may be a full-length antibody or an antibody fragment. Antibody fragments are smaller fragments of a full-length antibody that have antigen-binding capacity. Some of the most commonly used antibody fragments include Fab (fragment antigen-binding) fragments, F(ab′)2 (fragment antigen-binding dimer) fragments, single-chain variable fragment (scFv), nanobodies, bispecific antibodies, diabodies, triabodies, and domain antibodies (dAbs). Fab fragments are the variable regions of the antibody that contain the antigen-binding site. Fab fragments can be produced by enzymatic cleavage of the antibody molecule and are often used in diagnostic applications, for example. F(ab′)2 fragments are the Fab fragments joined together by a disulfide bond, resulting in a fragment that can bind two antigen molecules simultaneously. Single-chain variable fragments are recombinant antibody fragments that include the variable regions of the heavy and light chains of an antibody connected by a short linker peptide. Single-chain variable fragments can be produced in bacteria or yeast and are often used for targeting tumors or other disease-related antigens, for example. Nanobodies are single-domain antibody fragments derived from camelid or shark antibodies that have a small size and high stability. Nanobodies can be produced by genetic engineering, for example. Bispecific antibodies are antibodies that can bind to two different antigens simultaneously. Bispecific antibodies can be produced by fusing two different Fab or scFv fragments together or by engineering a single antibody molecule to contain two different antigen-binding sites. Diabodies are artificially engineered antibodies consisting of two different single-chain variable fragments (scFv) joined together. Diabodies have a small size and can bind to two different antigens simultaneously. Triabodies are artificially engineered antibodies consisting of three different single-chain variable fragments (scFv) joined together. Triabodies have a small size and can bind to three different antigens simultaneously. Domain antibodies are antibody fragments consisting of a single variable domain of the antibody that can be produced in bacteria or yeast. dAbs have a small size and high stability.

In some embodiments, the therapeutic modality is a therapeutic antibody, such as a monoclonal antibody. Non-limiting examples of therapeutic antibodies include Trastuzumab (HERCEPTIN®): Rituximab (RITUXAN®), Bevacizumab (AVASTIN®), Pembrolizumab (KEYTRUDA®), Nivolumab (OPDIVO®), Atezolizumab (TECENTRIQ®), Durvalumab (IMFINZI®), Cetuximab (ERBITUX®), Panitumumab (VECTIBIX®), and Daratumumab (DARZALEX®). Trastuzumab is a monoclonal antibody that targets HER2, a protein that is overexpressed in some types of breast cancer, and is used to treat HER2-positive breast cancer. Rituximab is a monoclonal antibody that targets CD20, a protein found on the surface of B cells, and is used to treat B-cell non-Hodgkin lymphoma, chronic lymphocytic leukemia, and other B-cell malignancies. Bevacizumab is a monoclonal antibody that targets vascular endothelial growth factor (VEGF) and is used to treat certain types of cancer, including colorectal, lung, and kidney cancer. Pembrolizumab is a monoclonal antibody that targets programmed death receptor-1 (PD-1) and is used to treat certain types of cancer, including melanoma, lung cancer, and head and neck cancer. Nivolumab is a monoclonal antibody that also targets PD-1 and is used to treat certain types of cancer, including melanoma, lung cancer, and renal cell carcinoma. Atezolizumab is a monoclonal antibody that targets programmed death-ligand 1 (PD-L1) and is used to treat certain types of cancer, including bladder cancer and non-small cell lung cancer. Durvalumab is a monoclonal antibody that also targets PD-L1 and is used to treat certain types of cancer, including bladder cancer and non-small cell lung cancer. Cetuximab is a monoclonal antibody that targets the epidermal growth factor receptor (EGFR) and is used to treat certain types of cancer, including head and neck cancer and colorectal cancer. Panitumumab is a monoclonal antibody that also targets EGFR and is used to treat colorectal cancer. Daratumumab is a monoclonal antibody that targets CD38, a protein found on the surface of multiple myeloma cells, and is used to treat multiple myeloma.

Small molecule drugs are low molecular weight (e.g., less than 10 kDa) compounds that can bind to and modify the activity of specific proteins in the body. Small molecule drugs are often used to treat diseases such as cancer, hypertension, and diabetes, for example. Non-limiting examples of small molecule drugs that may be used as a therapeutic modality include Imatinib (GLEEVEC®), Erlotinib (TARCEVA®), Sorafenib (NEXAVAR®), Everolimus (AFINITOR®), Crizotinib (XALKORI®), Venetoclax (VENCLEXTA®), Olaparib (LYNPARZA®), Enzalutamide (XTANDI®), Ibrutinib (IMBRUVICA®), and Palbociclib (IBRANCE®). Imatinib is a tyrosine kinase inhibitor that is used to treat chronic myeloid leukemia (CML) and some types of gastrointestinal stromal tumors (GIST). Erlotinib is a tyrosine kinase inhibitor that is used to treat non-small cell lung cancer (NSCLC) that has a specific mutation in the epidermal growth factor receptor (EGFR). Sorafenib is a tyrosine kinase inhibitor that is used to treat advanced renal cell carcinoma (RCC) and some types of liver cancer. Everolimus is a mammalian target of rapamycin (mTOR) inhibitor that is used to treat advanced RCC and some types of breast cancer. Crizotinib is a tyrosine kinase inhibitor that is used to treat NSCLC that has a specific mutation in the anaplastic lymphoma kinase (ALK) gene. Venetoclax is a B-cell lymphoma-2 (BCL-2) inhibitor that is used to treat chronic lymphocytic leukemia (CLL) and some types of lymphoma. Olaparib is a poly ADP-ribose polymerase (PARP) inhibitor that is used to treat some types of ovarian and breast cancer that have specific mutations in the BRCA genes. Enzalutamide is an androgen receptor inhibitor that is used to treat advanced prostate cancer. Ibrutinib is a Bruton's tyrosine kinase (BTK) inhibitor that is used to treat some types of leukemia and lymphoma. Palbociclib is a cyclin-dependent kinase (CDK) 4/6 inhibitor that is used to treat some types of breast cancer.

Gene therapies involve the delivery of genetic material, such as DNA or RNA, to cells in the body to correct genetic defects or modify cellular function, for example. In cancer, gene therapy can be used to modify cancer cells or immune cells to help them better target and destroy cancer cells. Non-limiting examples of some of the most commonly studied gene therapies used to treat cancer include CAR T-cell therapy, oncolytic virus therapy, tumor suppressor gene therapy, suicide gene therapy, gene editing therapy, RNA interference (RNAi) therapy, T-cell receptor (TCR) gene therapy, NK cell therapy, and immune checkpoint inhibitor gene therapy. CAR T-cell therapy is a type of gene therapy that involves modifying a patient's own T cells to express a chimeric antigen receptor (CAR) that can recognize and attack cancer cells. CAR T-cell therapy has been approved for the treatment of certain types of leukemia and lymphoma. Oncolytic virus therapy is a type of gene therapy that involves using viruses that have been modified to selectively infect and kill cancer cells. Oncolytic viruses can also be engineered to express genes that stimulate the immune system to attack cancer cells. Tumor suppressor gene therapy involves introducing genes that encode tumor suppressor proteins, such as p53, into cancer cells to help inhibit their growth and survival. Suicide gene therapy involves introducing genes that can cause cancer cells to self-destruct, such as the herpes simplex virus thymidine kinase (HSV-TK) gene, which can be activated by a prodrug called ganciclovir. Gene editing therapy involves using technologies such as CRISPR/Cas9 to selectively modify the genes in cancer cells to help inhibit their growth and survival. RNA interference (RNAi) therapy involves using small RNA molecules to selectively silence specific genes that are involved in cancer growth and progression. T-cell receptor (TCR) gene therapy involves modifying a patient's own T cells to express a TCR that can recognize and attack cancer cells. NK cell therapy involves using natural killer (NK) cells, a type of immune cell, that have been genetically modified to express chimeric antigen receptors (CARs) or other genes that enhance their ability to recognize and attack cancer cells. Immune checkpoint inhibitor gene therapy involves introducing genes that encode immune checkpoint inhibitors, such as PD-1 or CTLA-4, into immune cells to help enhance their ability to attack cancer cells.

Cell therapies involve the transplantation or modification of cells in the body to replace damaged or diseased cells or tissues, for example. Non-limiting examples of cell therapies include stem cell therapy, CAR T-cell therapy, gene editing using CRISPR/Cas9, mesenchymal stem cell therapy, retinal pigment epithelial cell therapy, natural killer cell therapy, tumor-infiltrating lymphocyte therapy, dendritic cell therapy, cord blood stem cell therapy, and tissue engineering. Stem cell therapy involves the transplantation of stem cells, which can differentiate into various cell types in the body, to replace or regenerate damaged or diseased tissues. CAR T-cell therapy involves the modification of a patient's own T cells to express chimeric antigen receptors (CARs) that can recognize and eliminate cancer cells. Gene editing using CRISPR/Cas9 and other endonuclease-based system that involve the modification of the DNA sequence of cells to correct genetic defects or modify cellular function, for example. Mesenchymal stem cell therapy involves the use of mesenchymal stem cells, which have anti-inflammatory and immunomodulatory properties, to treat inflammatory and autoimmune disorders. Retinal pigment epithelial cell therapy involves the use of retinal pigment epithelial cells to treat age-related macular degeneration. Natural killer cell therapy involves the use of natural killer cells, which can recognize and kill cancer cells and infected cells, to treat cancer and viral infections. Tumor-infiltrating lymphocyte (TIL) therapy involves the isolation and expansion of tumor-infiltrating lymphocytes, which are immune cells that have infiltrated a tumor, and their reinfusion into the patient to enhance the anti-tumor immune response. Dendritic cell therapy involves the isolation and activation of dendritic cells, which are immune cells that can stimulate an immune response, and their use as a cancer vaccine. Cord blood stem cell therapy involves the use of stem cells isolated from umbilical cord blood, which have the ability to differentiate into various cell types in the body, to treat diseases such as leukemia and sickle cell anemia. Tissue engineering involves the use of cells, biomaterials, and growth factors to create functional tissues or organs in the laboratory for transplantation into the patient.

Vaccines are biological preparations that stimulate the immune system to produce a protective immune response against a specific infectious agent, such as a virus or bacteria. There are several types of vaccines, each of which uses a different method to stimulate the immune response. Some of the most common types of vaccines include inactivated vaccines, live attenuated vaccines, subunit, recombinant, and conjugate vaccines, mRNA vaccines, viral vector vaccines, and DNA vaccines. Inactivated vaccines contain killed or inactivated pathogens that cannot cause disease but can still stimulate the immune system to produce an immune response. Examples of inactivated vaccines include the polio vaccine and the hepatitis A vaccine. Live attenuated vaccines contain weakened, but still live, pathogens that can stimulate the immune system to produce a strong and long-lasting immune response.

Examples of live attenuated vaccines include the measles, mumps, and rubella (MMR) vaccine and the yellow fever vaccine. Subunit, recombinant, and conjugate vaccines contain specific parts of the pathogen, such as proteins or sugars, that can stimulate the immune system to produce an immune response. Examples of subunit vaccines include the human papillomavirus vaccine and the hepatitis B vaccine. Recombinant vaccines use genetically engineered proteins or particles to stimulate an immune response, while conjugate vaccines combine bacterial proteins with sugars to stimulate an immune response. mRNA vaccines use fragments of messenger RNA (mRNA) to instruct cells to produce a specific protein (e.g., antigen) from the pathogen, which can then stimulate the immune system to produce an immune response. Examples of mRNA vaccines include the Moderna COVID-19 vaccine and the Pfizer-BioNTech COVID-19 vaccine. Viral vector vaccines use a harmless virus, such as an adenovirus or a poxvirus, to deliver genetic material from the pathogen into cells, which can then produce a specific protein from the pathogen and stimulate an immune response. Examples of viral vector vaccines include the Johnson & Johnson COVID-19 vaccine and the Ebola vaccine. DNA vaccines use fragments of DNA from the pathogen to stimulate an immune response. The DNA is usually delivered using a harmless virus or by direct injection.

Hormones are chemical messengers produced by the endocrine system that regulate various physiological functions in the body. Hormones can be used as therapeutics to treat a variety if conditions such as diabetes, thyroid disorders, and growth hormone deficiency. Non-limiting examples of therapeutic hormones include insulin, thyroid hormone, growth hormone, adrenocorticotropic hormone (ACTH), gonadotropins, estrogen and progesterone, androgens, cortisol, parathyroid hormone, and vasopressin.

Enzyme replacement therapies involve the administration of enzymes to replace or supplement enzymes that are deficient or missing in the body. Enzyme replacement therapy is often used to treat lysosomal storage disorders, such as Gaucher disease and Fabry disease.

Nucleic acid-based therapies involve the delivery of nucleic acids, such as DNA or RNA, to cells in the body to modify gene expression or cellular function. Nucleic acid-based therapies include antisense oligonucleotide therapy and RNA interference therapy. RNA interference (RNAi) therapy is a type of gene therapy that involves the use of small RNA molecules to silence or “knock down” the expression of specific genes in the body. Examples of types of RNAi molecules include short interfering RNA (siRNA), microRNA (miRNA), short hairpin RNA (shRNA), ribozymes, aptamers, antisense RNA, and CRISPR RNA (crRNA). Short interfering RNA is a double-stranded RNA molecule that is typically 21-23 nucleotides in length. siRNA molecules are used to silence specific genes by targeting their mRNA for degradation. MicroRNAs are small, single-stranded RNA molecules that are typically 20-24 nucleotides in length. miRNA molecules are involved in regulating the expression of multiple genes by targeting their mRNAs for degradation or translation inhibition. Short hairpin RNA is a single-stranded RNA molecule that is typically 19-29 nucleotides in length and folds back on itself to form a hairpin structure. shRNA molecules are used to silence specific genes by targeting their mRNA for degradation. Ribozymes are RNA molecules that have enzymatic activity and can cleave specific RNA molecules, including mRNA. Aptamers are RNA molecules that can bind to specific targets, such as proteins or other molecules, with high affinity and specificity. Antisense RNA is a single-stranded RNA molecule that is complementary to a specific mRNA molecule. Antisense RNA molecules are used to inhibit translation of the target mRNA by forming a double-stranded RNA molecule that is degraded by the cell. CRISPR RNA is a RNA molecule that is part of the CRISPR-Cas9 system, a genome editing tool that can be used to target specific genes for deletion or modification.

Some of the most commonly studied RNAi therapies include Patisiran (ONPATTRO®), Inclisiran (LEQVIO®), Lumasiran (OXLUMO®), Givosiran (GIVLAARI®), Fitusiran, ALN-TTRsc02, RG6346, Danvatirsen, QPI-1007, and IONIS-TTR-LRx. Patisiran is an RNAi therapeutic approved by the FDA for the treatment of hereditary transthyretin-mediated (hATTR) amyloidosis, a rare and progressive genetic disorder that causes the accumulation of amyloid fibrils in various organs. Inclisiran is an RNAi therapeutic approved by the FDA for the treatment of high cholesterol. It targets PCSK9, a protein that regulates the levels of low-density lipoprotein (LDL) cholesterol in the blood. Lumasiran is an RNAi therapeutic approved by the FDA for the treatment of primary hyperoxaluria type 1 (PH1), a rare genetic disorder that causes the buildup of oxalate in the kidneys and other organs. Givosiran is an RNAi therapeutic approved by the FDA for the treatment of acute hepatic porphyria (AHP), a group of rare genetic disorders that affect the production of heme, a component of hemoglobin. Fitusiran is an RNAi therapeutic currently in clinical trials for the treatment of hemophilia A and B, rare bleeding disorders caused by deficiencies in clotting factors. ALN-TTRsc02 is an RNAi therapeutic currently in clinical trials for the treatment of hATTR amyloidosis. RG6346 is an RNAi therapeutic currently in clinical trials for the treatment of age-related macular degeneration, a leading cause of blindness in older adults. Danvatirsen is an RNAi therapeutic currently in clinical trials for the treatment of cancer, specifically non-small cell lung cancer and multiple myeloma. QPI-1007 is an RNAi therapeutic currently in clinical trials for the treatment of rare inherited eye diseases, including Leber hereditary optic neuropathy (LHON) and autosomal dominant optic atrophy (ADOA). IONIS-TTR-LRx is an RNAi therapeutic currently in clinical trials for the treatment of hATTR amyloidosis.

### Engineered Immune Cell Therapies

In some embodiments, the mouse models provided herein are used to assess efficacy and/or side effects (e.g., cytokine response) following administration of a cell therapy, such as a human immune cell therapy. In some embodiments, a human immune cell (e.g., used for therapy) comprises a receptor that specifically binds to a cell surface antigen on a diseased human cell. In some embodiments, the human immune cell is one that may be used in adoptive cell therapy (ACT). As used herein “adoptive cell therapy” or “ACT” refers to a cell-based immunotherapy that relates to the transfusion of autologous or allogenic immune cells, genetically modified or not, that have been expanded ex vivo prior to the transfusion. The human immune cells, in some embodiments, are engineered human immune cells. Any human immune cell that comprises an engineered T cell receptor is considered to be an “engineered” human immune cell.

In some embodiments, a human immune cell comprises an engineered T cell receptor (eTCR). As described herein, “eTCR” refers to a dimeric heterologous cell surface signaling protein forming an alpha-beta or gamma-delta receptor typically involved in recognizing an antigen presented by a major histocompatibility complex (MHC) molecule (i.e., antigen recognition in the context of an MHC molecule). This differs from CAR T cell therapy, in which antibody fragments that bind to specific surface antigens of cancer cells are used. In some embodiments, eTCRs are modified to target or recognize histocompatibility antigen 1 (HA1), Wilms tumor 1 (WT1), cytomegalovirus (CMV), melanoma antigen (MAGE), glycoprotein 100 (gp100), MAR-1, human papillomavirus-16 E6 protein (HPV-16 E6), New York esophageal squamous cell carcinoma (NY-ESO-1), hepatitis B virus (HBV), protein 53 (P53), carcinoembryonic antigen (CEA), HPV E7, HIVgag-specific peptide SLYNTVATL (SL9), transforming growth factor-beta 2 (TGFβ2), monocyte chemotactic protein (MCP [symbol] V), TNF-related apoptosis-inducing ligand (TRAIL), preferentially expressed antigen in melanoma (PRAME), Epstein-Barr virus (EBV), or Kirsten rat sarcoma virus (KRAS) (Zhao et al., Front. Immunol., 11 Oct. 2019). In some embodiments, a human immune cell is a T cell with an eTCR.

In some embodiments, a human immune cell is an engineered tumor-infiltrating lymphocyte (TIL). In TIL therapy (or engineered TIL therapy, eTIL therapy), TILs are removed from a subject's tumor (e.g., during a biopsy or surgical resection) and grown and expanded ex vivo with interleukin-2 (IL-2) and/or other cytokines. The TILs, which are naturally present in some tumors and are capable of recognizing and killing cancer cells, are then administered to the subject (e.g., by infusion). In some embodiments, the TILs are engineered TILs (eTILs), which have been modified to increase tumor homing ability, cytotoxicity and/or to improve longevity (prevent exhaustion) (Jimenez-Reinoso et al., Front. Oncol., 16 Feb. 2021). For example, in some embodiments, the eTILs may be transfected with TRAIL, IL-12, CXCL8, and/or CXCR2.

In some embodiments, a human immune cell is a regulatory T cell (Treg). As used herein, a Treg, also known as a suppressor T cell, is a subpopulation of T cells that modulate the immune system, maintain tolerance to self-antigens, and prevent autoimmune disease. Tregs are CD4⁺CD25⁺FoxP3⁺, immunosuppressive, and generally suppress or downregulate induction and proliferation of T effector cells. In some embodiments, administration of the Tregs may treat or prevent cancer. For example, it has been found that administration of Tregs downregulates inflammation, blocking the development of bacteria-triggered colitis and colorectal cancer (Poutahidis et al., Carcinogenesis. 2007 December;28 (12): 2614-23. doi: 10.1093/carcin/bgm180. Epub 2007 Aug. 27). In some embodiments, the Tregs comprise a chimeric antigen receptor (CAR) as described below (Mohseni et al., Front. Immunol., 24 July 2020).

It should be understood that while many embodiments describe herein are directed to assessing the effects of cell therapies for treating cancer, the disclosure is not so limited. The mouse models described herein may be used to assess a myriad of engineered immune cell therapies, particularly those associated with the induction of CRS. Thus, in some embodiments, the human immune cells are human T cells. In some embodiments, the human immune cells are human B cells. In some embodiments, the human immune cells are human NK cells. In some embodiments, the human immune cells are human CAR T cells (e.g., a CD8+ or a CD4+ T cell). In some embodiments, the human immune cells are human CAR B cells. In some embodiments, the human immune cells are human CAR NK cells. In some embodiments, the human immune cells are human eTCR T cells. In some embodiments, the human immune cells are human eTILs. In some embodiments, the human immune cells are human eTregs. Any one or more of the human immune cells may comprise an engineered receptor that specifically binds to a cell surface antigen on diseased human cells, for example, cancer cells, or other cells associated with cardiovascular disease, metabolic disease, or other pathological states. In some embodiments, a human immune cell comprises an engineered receptor that specifically binds to a cell surface antigen on a cancer cell. In some embodiments, a human immune cell comprises an engineered receptor that specifically binds to a cell surface antigen on a diseased cardiovascular cell. In some embodiments, a human immune cell comprises an engineered receptor that specifically binds to a cell surface antigen on a diseased metabolic cell.

### Chimeric Antigen Receptor (CAR) Immune Cell Therapies

A “chimeric antigen receptor” refers to an artificial immune cell receptor that is engineered to recognize and bind to an antigen expressed by diseased cells (e.g., tumor cells). Generally, a CAR is designed for a T cell and is a chimera of a signaling domain of the T cell receptor (TcR) complex and an antigen-recognizing domain (e.g., a single chain fragment (scFv) of an antibody or other antibody fragment) (Enblad et al., Human Gene Therapy. 2015; 26 (8): 498-505). A T cell that expresses a CAR is referred to as a “CAR T cell.” In some embodiments, the T cell is a Treg (CD4⁺CD25⁺FoxP3⁺) and resulting CAR T cell is referred to as a “CAR Treg cell.”

There are five generations of CARs, each of which contains different components. First generation CARs join an antibody-derived scFv to the CD3zeta ((or z) intracellular signaling domain of the T cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-ζ chain. Third-generation costimulatory domains may include, e.g., a combination of CD3z, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125 (26): 4017-4023; Kakarla and Gottschalk, Cancer J. 2014; 20 (2): 151-155).

In some embodiments, the chimeric antigen receptor (CAR) is a T cell redirected for universal cytokine killing (TRUCK), also known as a fourth generation CAR. TRUCKs are CAR-redirected T cells used as vehicles to produce and release a transgenic cytokine, IL-12, that accumulates in the targeted tissue, e.g., a targeted tumor tissue. The transgenic cytokine is released upon CAR engagement of the target. This may result in therapeutic concentrations at the targeted site and avoid systemic toxicity.

In some embodiments, the CAR T cell is a fifth generation CAR or next-generation CAR. Fifth generation CAR T cells are based on second generation CARs, having additional intracellular domains of cytokine receptors. In some embodiments, the additional intracellular domain is a cytoplasmic IL-2 receptor (e.g., IL-2Rβ having a STAT3/5 binding motif), which is a binding site for STAT3/5, a transcription factor (Tokarew et al., British Journal of Cancer. 2019; 120:26-37). By including the binding site, the CAR is capable of producing all three synergistic signals necessary to physiologically to drive full T cell activation and proliferation: TCR (through the CD3ζ domains), co-stimulatory (CD28 domain), and cytokine (JAK-STAT3/5) signaling.

CARs typically differ in their functional properties. The CD3ζ signaling domain of the T cell receptor, when engaged, will activate and induce proliferation of T cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T cell to modulate the T cell response. See, for example, Enblad et al., Human Gene Therapy. 2015; 26 (8): 498-505; Chmielewski and Hinrich, Expert Opinion on Biological Therapy. 2015;15 (8): 1145-1154. As noted above, fifth generation CARs further comprise cytokine receptor domains and are able to trigger cytokine signaling, further enhancing T cell proliferation and maintenance (Tokarew et al., British Journal of Cancer. 2019; 120:26-37).

Other immune cells may be reprogramed using CAR technology. For example, NK cells, B cells, dendritic cells, monocytes/macrophages, and neutrophils may also be reprogramed to expression at least one CAR.

NK cells are derived from the bone marrow and defend against viruses and prevent cancer. These cells can kill cells (e.g., virus-infected cells) by injecting a combination of chemicals lethal to the cell. They have been investigated for cancer immunotherapy (Xie et al., EBioMedicine, 2020, 59:102975; Wang et al., Cancer Letters, 2020, 472:175-180; Pfefferle et al., Cancers (Basal), 2020, 12 (3): 76; Habib et al., Ochsner Journal, 2019, 19 (3): 186-187).

B cells (B-lymphocytes) are immune cells that develop in the bone marrow from hematopoietic stem cells and produce antibodies. B cells are “trained” so that they do not produce antibodies against healthy tissue, and when they encounter foreign (non-self) material, they mature into plasma cells or memory cells.

Dendritic cells are antigen-presenting cells that process antigen material and present it on their respective cell surfaces to T cells. In this way, they act as a liaison between the innate and adaptive immune systems. CAR dendritic cells (CAR-DC) have been used in conjunction with CAR T cells to improve anti-cancer cytotoxicity (Suh et al., Journal of Clinical Oncology, 2018, 35 (7): 144).

Monocytes (macrophages) are phagocytic cells of the immune system found in all tissues. They play a role in both adaptive and innate immunity, and in some instances, work with T cells to kill microorganisms. CAR macrophages (CAR-M) have been shown to reduce or eliminate tumor cells in an ovarian cancer cell line (Klichinsky et al., Nature Biotechnology, 2020, 38:947-53).

Neutrophils (polymorphonuclear leukocytes, granulocytes) also develop in the bone marrow, and leave the blood stream to accumulate in infected tissues. Typically, during the acute phase of an infection, a neutrophil will migrate to the site of inflammation, where they ingest organisms to kill them.

In some embodiments, the engineered immune cell (e.g., T cell, B cell, or NK cell) therapy is autologous; that is, for example, a subject's T cells are collected and used to generate the CAR T cells that are later used to treat the subject.

In some embodiments, the engineered immune cells are universal allogeneic engineered immune cells (e.g., “off-the-shelf” engineered immune cells). Allogeneic engineered immune cells use donor immune cells; that is, immune cells from a source other than the subject (recipient) who undergoes the engineered immune cell (e.g., T cell, B cell, or NK cell) therapy. In some embodiments, the donor immune cells are from a healthy human (e.g., adult or child). Allogeneic engineered immune cells (e.g., CAR T cells) can cause graft-versus-host disease (GVHD) in a subject after administration, for example, if the engineered immune cell recognizes cell surface HLA class I and class II molecules on the subject's cells as “non-self” and attacks them. In order to circumvent this issue with respect to T cells, the T cell αβ receptor (TCRαβ) of the CAR T cell may be knocked out using gene editing tools (e.g., zinc finger nucleases, transcription activator like effector nucleases, or clustered regularly interspaced short palindromic repeats (CRISPR)/CRISPR-associated protein 9 (Cas9)) (Kim et al., Biomolecules. 2020; 10 (2): 263). In this way, universal allogeneic CAR T cells may be administered to any subject (recipient).

In some embodiments, a chimeric antigen receptor (CAR) comprises an extracellular domain comprising an antigen binding domain, a transmembrane domain, and a cytoplasmic domain. In some embodiments, a CAR is fully human. In some embodiments, the antigen binding domain of a CAR is specific for one or more antigens. In some embodiments, a “spacer” domain or “hinge” domain is located between an extracellular domain (comprising the antigen binding domain) and a transmembrane domain of a CAR, or between a cytoplasmic domain and a transmembrane domain of the CAR. A “spacer domain” refers to any oligopeptide or polypeptide that functions to link the transmembrane domain to the extracellular domain and/or the cytoplasmic domain in the polypeptide chain. A “hinge domain” refers to any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR of the disclosure comprises an antigen binding domain, such as a single chain Fv (scFv) specific for an antigen (e.g., a tumor antigen). The choice of binding domain depends upon the type and number of ligands that define the surface of a cell. For example, the antigen binding domain may be chosen to recognize a ligand that acts as a cell surface marker on cells associated with a particular disease state, such as cancer, cardiovascular disease, metabolic disease, neurobiological disease, or an autoimmune disease. Thus, examples of cell surface markers that may act as ligands for the antigen binding domain in the CAR of the present disclosure include those associated with cancer cells and/or other forms of diseased cells. In some embodiments, a CAR is engineered to target a tumor antigen of interest by way of engineering a desired antigen binding domain that specifically binds to a surface antigen of an Epstein-Barr virus (EBV) or papillomavirus particle.

An antigen binding domain (e.g., an scFv) that “specifically binds” to a target or an epitope is a term understood in the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antigen binding domain (e.g., an scFv) that specifically binds to a first target antigen may or may not specifically bind to a second target antigen. As such, “specific binding” does not necessarily require (although it can include) exclusive binding.

In some embodiments, immune cells expressing a CAR are genetically modified to recognize multiple targets or antigens, which permits the recognition of unique target or antigen expression patterns on diseased cells (e.g., tumor cells). Examples of CARs that can bind multiple targets include: “split signal CARs,” which limit complete immune cell activation to tumors expressing multiple antigens; “tandem CARs” (TanCARs), which contain ectodomains having two scFvs; and “universal ectodomain CARs,” which incorporate avidin or a fluorescein isothiocyanate (FITC)-specific scFv to recognize tumor cells that have been incubated with tagged monoclonal antibodies (mAbs).

In some embodiments, the target is CD19; that is, the CAR is a CD19 CAR. In some embodiments, the target is CD22; that is, the CAR is a CD22 CAR. In some embodiments, the target is CD123; that is, the CAR is a CD123 CAR. Other targets include, but are not limited to CD20, B cell maturation antigen (BCMA), C-type lectin-like molecule-1 (CLL-1), tyrosine-protein kinase transmembrane receptor 1 (ROR-1), IL13Ra2, CD20, CD138, CD33, prostate specific membrane antigen (PSMA), CD171, epidermal growth factor receptor variant III (EGFRvIII), fibroblast activation protein (FAP), folate receptor (FR), glypican-3, human epidermal growth factor receptor 2 (HER2), mucin 1, cell surface associated (MUC1), mesothelin, and natural killer group 2D (NKG2D). In some embodiments, the CAR is specific to any target or antigen of interest that is found on the surface of a cancer cell. In other embodiments, the CAR is specific to any target or antigen of interest that is found on the surface of a diseased cell.

A CAR is considered “bispecific” if it recognizes two distinct antigens (has two distinct antigen recognition domains). In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013;2: e105, incorporated herein by reference).

In some embodiments, a CAR is an antigen-specific inhibitory CAR (iCAR), which may be used, for example, to avoid off-tumor toxicity (Fedorov, VD et al. Sci. Transl. Med. published online Dec. 11, 2013, incorporated herein by reference). iCARs contain an antigen-specific inhibitory receptor, for example, to block nonspecific immunosuppression, which may result from extratumor target expression. iCARs may be based, for example, on inhibitory molecules CTLA-4 or PD-1, to block immunosuppression, or on a pan-leukocyte antigen, such as CD52, to block leukocyte destruction. In some embodiments, these iCARs block T cell responses from T cells activated by either their endogenous T cell receptor or an activating CAR.

In some embodiments, CARs are engineered for use in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5 (3): 211-226; Maude et al., Blood. 2015; 125 (26): 4017-4023, each of which is incorporated herein by reference).

### Antibody and Other Recombinant Protein Therapies

In some embodiments, the mouse models provided herein are used to assess efficacy and/or side effects (e.g., cytokine response) following administration of an antibody therapy or other recombinant protein therapy.

“Antibody” encompasses antibodies or immunoglobulins of any isotype, including but not limited to humanized antibodies and chimeric antibodies. An antibody may be a single-chain antibody (scAb) or a single domain antibody (dAb) (e.g., a single domain heavy chain antibody or a single domain light chain antibody; see Holt et al. (2003) Trends Biotechnol. 21:484). The term “antibody” also encompasses fragments of antibodies (antibody fragments) that retain specific binding to an antigen. “Antibody” further includes single-chain variable fragments (scFvs), which are fusion proteins of the variable regions of the heavy (VH) and light chains (VL) of antibodies, connected with a short linker peptide, and diabodies, which are noncovalent dimers of scFv fragments that include the VH and VL connected by a small peptide linker (Zapata et al., Protein Eng. 8 (10): 1057-1062 (1995)). Other fusion proteins that comprise an antigen-binding portion of an antibody and a non-antibody protein are also encompassed by the term “antibody.”

“Antibody fragments” comprise a portion of an intact antibody, for example, the antigen binding or variable region of the intact antibody. Examples of antibody fragments include an antigen-binding fragment (Fab), Fab′, F(ab′)₂, a variable domain Fv fragment (Fv), an Fd fragment, and an antigen binding fragment of a chimeric antigen receptor.

Papain digestion of antibodies produces two identical antigen-binding fragments, referred to as “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and-binding site. This region includes a dimer of one heavy-chain variable domain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” fragments contain the constant domain of the light chain and the first constant domain (CH₁) of the heavy chain. Fab fragments differ from Fab′ fragments by the addition of a few residues at the carboxyl terminus of the heavy chain CH₁ domain including at least one cysteine from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“scFv” antibody fragments comprise the V_{H} and V_{L} of an antibody, wherein these regions are present in a single polypeptide chain. In some cases, the Fv polypeptide further comprises a polypeptide linker between the V_{H} and V_{L} regions, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

“Diabody” refers to a small antibody fragment with two antigen-binding sites, which fragments comprise a V_{H} connected to a V_{L} in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, Hollinger et al. Proc. Natl. Acad. Sci. USA 90:6444-6448 (1993).

An antibody can be monovalent or bivalent. An antibody can be an Ig monomer, which is a “Y-shaped” molecule that consists of four polypeptide chains: two heavy chains and two light chains connected by disulfide bonds.

Antibodies can be detectably labeled, e.g., with a radioisotope, an enzyme that generates a detectable product, and/or a fluorescent protein. Antibodies can be further conjugated to other moieties, such as members of specific binding pairs, e.g., biotin member of biotin-avidin specific binding pair. Antibodies can also be bound to a solid support, including, but not limited to, polystyrene plates and/or beads.

An “isolated” antibody is one that has been identified and separated and/or recovered from a component of its natural environment (i.e., is not naturally occurring). Contaminant components of its natural environment are materials that would interfere with uses (e.g., diagnostic or therapeutic uses) of the antibody, and can include enzymes, hormones, and other proteinaceous or nonproteinaceous solutes. In some cases, an antibody is purified (1) to greater than 90%, greater than 95%, or greater than 98% by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or non-reducing conditions using Coomassie blue or silver stain. Isolated antibodies encompass antibodies in situ within recombinant cells, as at least one component of the antibody's natural environment will not be present. In some embodiments, an isolated antibody is prepared by at least one purification step.

A “monoclonal antibody” is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

A “complementarity determining region (CDR)” is the non-contiguous antigen combining sites found within the variable region of both heavy and light chain polypeptides. CDRs have been described by Lefranc et al. (2003) Developmental and Comparative Immunology 27:55; Kabat et al., J. Biol. Chem. 252:6609-6616 (1977); Kabat et al., U. S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1,” “CDR-L2,” and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

A “framework” when used in reference to an antibody variable region includes all amino acid residues outside the CDR regions within the variable region of an antibody. A variable region framework is generally a discontinuous amino acid sequence that includes only those amino acids outside of the CDRs. A “framework region” includes each domain of the framework that is separated by the CDRs.

A “humanized antibody” is an antibody comprising portions of antibodies of different origin, wherein at least one portion comprises amino acid sequences of human origin. For example, the humanized antibody can comprise portions derived from an antibody of nonhuman origin with the requisite specificity, such as a mouse, and from antibody sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing at least one chain comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242:423-426 (1988)), regarding single chain antibodies.

In some embodiments, a humanized antibody is produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17:5404 (1989)); Sato, K., et al., Cancer Research, 53:851-856 (1993); Daugherty, B. L. et al., Nucleic Acids Res., 19 (9): 2471-2476 (1991); and Lewis, A. P. and J. S. Crowe, Gene, 101:297-302(1991)). Using these or other suitable methods, variants can also be readily produced. For example, cloned variable regions can be mutagenized, and sequences encoding variants with the desired specificity can be selected (e.g., from a phage library; see e.g., Krebber et al., U. S. Pat. No. 5,514,548; Hoogenboom et al., WO 93/06213, published Apr. 1, 1993).

Other non-limiting examples of recombinant protein-based therapies include antibody-drug conjugates, PEGylated proteins, albumin fusion proteins, and Fc fusion proteins.

### Nucleic Acid-Based Therapies

In some embodiments, the mouse models provided herein are used to assess efficacy and/or side effects (e.g., cytokine response) following administration of nucleic acid-based therapy. Non-limiting examples of nucleic acid-based therapies include antisense oligonucleotides (ASOs), siRNA and mRNA therapies (e.g., delivered via lipid nanoparticle (LNP)), and viral gene therapies (e.g., adeno-associated viral (AAV) and lentiviral therapies). See, e.g., Kulkarni J A et al. Nature Nanotechnology 16, pages 630-643 (2021), incorporated herein by reference.

ASOs are short synthetic nucleic acids that hybridize with cellular RNA using classic Watson-Crick base pairing to modulate gene expression. By binding pre-mRNA or mRNA, ASOs can post-transcriptionally regulate protein synthesis through mechanisms including modification of pre-mRNA processing and splicing, competitive inhibition, steric blockade of translational machinery, and degradation of bound target RNA. The latter mechanism leverages endogenous RNA degradation pathways mediated by RNase H1 (recruited by DNA: RNA duplexes) or Argonaute 2 (recruited by RNA: RNA duplexes through the RNA interference pathway). The ability to interact with pre-mRNA enables ASOs to target splicing processes and dramatically increases the amount of RNA sequence that can be selected for ASO binding, which can also limit off-target effects.

GalNAc conjugation represents an efficient way of increasing siRNA target organ accumulation and of facilitating their cellular uptake. In the absence of a protective delivery vector, siRNA has to be chemically modified to ensure stability in the circulation following parenteral administration. To silence disease-causing genes in hepatocytes, these therapeutics are composed of siRNA conjugated to a triantennary GalNAc moiety targeting the asialoglycoprotein receptor (ASGPR). This receptor is predominantly expressed on hepatocytes and thus provides access to a defined cell type within the liver. ASGPR specifically binds carbohydrates with terminal galactose or GalNAc residues43. After ligand binding, the receptor-ligand complex is internalized by clathrin-dependent receptor-mediated endocytosis. Abundant (around 500,000 ASGPR per cell) and predominantly expressed (>95% of total expression) on the hepatocyte sinusoidal membrane, ASGPR is an ideal receptor for hepatic siRNA delivery. Further, its high internalization and recycling rate (within minutes) allow continuous uptake of siRNA molecules, thereby increasing cell concentration. Different species exhibit the same carbohydrate recognition pattern, which is an important consideration in preclinical and translational study design.

RNA therapeutics are a class of medications based on ribonucleic acid (RNA). The main types of RNA therapeutics are those based on messenger RNA (mRNA), antisense RNA (asRNA), RNA interference (RNAi), and RNA aptamers. Of the four types, mRNA-based therapy is the only type which is based on triggering synthesis of proteins within cells, making it particularly useful in vaccine development. Antisense RNA is complementary to coding mRNA and is used to trigger mRNA inactivation to prevent the mRNA from being used in protein translation. RNAi-based systems use a similar mechanism, and involve the use of both small interfering RNA (siRNA) and micro RNA (miRNA) to prevent mRNA translation. RNA aptamers are short, single stranded RNA molecules produced by directed evolution to bind to a variety of biomolecular targets with high affinity thereby affecting their normal in vivo activity.

Wild-type AAV is a small, non-enveloped parvovirus (˜25 nm) with a ˜4.7-kb single-stranded DNA (ssDNA) genome. Recombinant AAV vectors exploited as delivery vehicles generally contain the same capsid components and structure as wild-type AAV but all viral coding sequences are replaced by therapeutic gene expression cassettes, maximizing packing capacity and reducing immunogenicity. Because AAV tropism has broad range, AAVs can be used for targeted transduction in a variety of tissues, potentially providing cellular specificity. Variable regions within capsid protein sequences define AAV serotypes and dictate tropism by modifying the binding interactions between capsids and receptors that facilitate their cellular uptake. Receptor-bound AAVs enter cells by endocytosis, are released into the cytosol by acidification of the early endosome, and, following perinuclear accumulation, enter the nucleus. Tissue specificity and transduction efficiency can be further modified by viral pseudotyping, chimeric capsid engineering and peptide library screening, among others.

### Small Molecule Drug Therapies

In some embodiments, the mouse models provided herein are used to assess efficacy and/or side effects (e.g., cytokine response) following administration of a small molecule drug therapy. Small-molecule drugs are chemical compounds with a molecular weight in the range of 0.1-1 kDa. They are smaller than biologics or bio-therapeutic modalities, which are generally more than 1 kDa in molecular size. Owing to the small size, they possess an advantage over biologics to target not only the extracellular components like cell surface receptors or protein domains attached to the cell membranes like glycoproteins but also the intracellular proteins like different kinases, as they can easily cross the outer plasma membrane of the cell.

### Cytokine Release Syndrome

Cytokine release syndrome (CRS) occurs with activation of T cells and Natural Killer (NK) cells as well as other immune cell populations (e.g., macrophages). With the addition of engineered immune cells (e.g., T cells, B cells, or NK cells), the activation of immune cells can lead to the release of high levels of cytokines and downstream injury and possibly death. T cells, NK cells, monocytes, and myeloid cells have been found to be sources of CRS in response to certain immunomodulators (Wing M. G. et al. (1995) Ther. Immunol. 2:183-190; Carson W. E., (1999) J Immunol 162;4943-4951). With different immunomodulators and the activation of various immune cell populations, CRS can manifest with high levels of cytokine release that can vary with the various activated immune cell populations.

The main cytokines associated with pathogenesis of CRS include interleukin-6 (IL-6), interleukin-10 (IL-10), interferon (IFN)-γ, monocyte chemoattractant protein 1 (MCP-1) and granulocyte-macrophage colony-stimulating factor (GM-CSF). Several other cytokines, including TNF, IL1, IL2, IL-2-receptor-a, and IL8 have also been associated with CRS. Several factors contributing to CRS toxicity in cancer patients, particularly those undergoing engineered immune cell (e.g., T cell, B cell, or NK cell) therapy include: the structure of the CAR, high tumor burden, higher immune cell infusion dose, and other patient-specific factors such as pre-existent state of inflammation and baseline endothelial activation.

One approach for preventing life-threatening high-grade CRS toxicity is to administer an anti-cytokine therapy early in CRS development (e.g., when a subject first exhibits symptoms of CRS). Tocilizumab (an IL6 antagonist), for example, has been approved by U.S. Food and Drug Administration (FDA) for the treatment of severe or life-threatening CAR T cell-induced CRS. Other treatments for CAR T cell-induced CRS include anti-IL-6 antibodies (e.g., siltuximab), corticosteroids (e.g., methylprednisone), anti-TNF-α drugs (e.g., etanercept), IL-1R inhibitors (e.g., anakinra), GM-CSF inhibitors, and small molecule inhibitors (e.g., ruxolitinib (JAK 1/2 inhibitor) and Bruton's tyrosine kinase inhibitor). In some embodiments, the mouse models provided herein are used to assess the therapeutic efficacy and/or side effects associated with candidate CRS treatments, for example, in subjects undergoing engineered immune cell (e.g., T cell, B cell, or NK cell) therapy, as described in more detail elsewhere herein.

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat, and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The first Lab Code appended to a strain symbol identifies and credits the creator of the strain. The Lab Code at the end of a strain symbol indicates the current source for obtaining mice of that strain. Different Lab Codes appended to the same strain symbol distinguish sublines and alert the user that there may be genetic divergence between the different sublines. Lab Codes are assigned from a central registry to assure that each is unique. The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson MT, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

A mouse model of disease may be modified to enable the assessment of a disease or a therapy targeting the disease. Any system (e.g., immune, respiratory, nervous, or circulatory), organ (e.g., blood, heart, blood vessels, spleen, thymus, lymph nodes, or lungs), tissue (e.g., epithelial, connective, muscle, or nervous), or cell type (e.g., lymphocytes or macrophages) may be modified, either independently or in combination, to enable studying disease or a therapy targeting the disease in the models provided herein.

Three conventional methods used for the production of genome-modified mice (e.g., knockout mice, transgenic mice) include DNA microinjection (Gordon and Ruddle, Science 1981:214: 1244-124), embryonic stem cell-mediated gene transfer (Gossler et al., Proc. Natl. Acad. Sci. 1986, 83:9065-9069) and retrovirus-mediated gene transfer (Jaenisch, Proc. Natl. Acad. Sci. 1976, 73:1260-1264), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) may also be used.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring.

New mouse models can also be created by breeding parental lines. With the variety of available mutant, knockout, knockin, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. An allele is one of two or more alternative forms of a gene that arise by mutation and are found at the same location on a chromosome. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise diploid organism in which both copies of the gene are missing. The mice described herein may by homozygous, heterozygous, hemizygous, or homozygous null for any one or more of the alleles described herein.

### Immunodeficient Mouse Models

Provided herein, in some embodiments, are immunodeficient mouse strains. Immunodeficient mouse strains are genetically engineered mice that have impaired or disrupted immune systems, making them useful models for studying human diseases and/or developing new therapies. These mice lack one or more key components of the immune system, such as T cells, B cells, and/or natural killer cells, which makes them unable to mount a proper immune response to infections or foreign substances. These mouse strains are commonly used in preclinical research to study cancer, infectious diseases, autoimmune disorders, and transplant rejection. For example, human tumor cells can be transplanted into immunodeficient mice to study cancer biology and test new cancer therapies. Immunodeficient mice can also be used to study infectious diseases by infecting them with human pathogens, such as viruses. Some immunodeficient mice have specific deficiencies in MHC class I and/or II or defects in the production and/or function of cells selected from B cells, T cells, natural killer (NK) cells, myeloid cells (e.g., granulocytes and/or monocytes), macrophage cells, and dendritic cell. Some immunodeficient mice also, or alternatively, have immunodeficiency due to knockdown of certain genes coding for cytokines, cytokine receptors, TLR receptors, and/or a variety of transducers and/or transcription factors of signaling pathways. Examples of immunodeficiency mouse models include the single-gene mutation models such as nude-mice (nu) strains, the severe combined immunodeficiency (scid) strains, non-obese diabetic (NOD) strains, RAG (recombination activating gene) strains with targeted gene deletion and a variety of hybrids originated by crossing doubly and triple mutation mice strains with additional defects in innate and adaptive immunity.

In some embodiments, an immunodeficient mouse comprises an interleukin-2 receptor gamma null (IL-2R [symbol] ^{null}) allele. An IL-2R [symbol] ^{null} allele is a null mutation in the gene encoding the interleukin 2 receptor gamma chain (IL2Rγ, homologous to IL2RG in humans), which blocks natural killer (NK) cell differentiation, thereby removing an obstacle that prevents the efficient engraftment of primary human cells (Cao et al., 1995; Greiner et al., 1998; and Shultz et al., 2005, each of which is incorporated herein by reference). In some embodiments, an immunodeficient mouse is homozygous for an IL-2R [symbol] ^{null} allele.

In some embodiments, an immunodeficient mouse comprises a Prkdc^{scid} allele. The Prkdc^{scid} mutation is a loss-of-function (null) mutation in the mouse homolog of the human PRKDC gene—this mutation essentially eliminates adaptive immunity (see, e.g., Blunt et al., 1995; Greiner, Hesselton, & Shultz, 1998), each of which is incorporated herein by reference). In some embodiments, an immunodeficient mouse is homozygous for a Prkdc^{scid} allele.

In some embodiments, an immunodeficient mouse comprises a Ragl^{null} allele. The Ragl^{null} mutation renders the mice B and T cell deficient. In some embodiments, an immunodeficient mouse is homozygous for a Ragl^{null} allele.

Non-limiting examples of spontaneous and transgenic immunodeficient mouse models include the following mouse strains:
NOD [Kikutani H et al. Adv Immunol 1992; 51:285-322; and Anderson M S et al. Ann Rev Immunol 2005; 23:447-85];
Nude (nu) [Flanagan S P. Genet Res 1966; 8:295-309; and Nehls M et al. Nature 1994; 372:103-7];
Scid (scid) [Bosma G C et al. Nature 1983; 301:527-30; Mosier DE et al. Nature 1988; 335:256-9; and Greiner D L et al. Stem Cells 1998; 16:166-77];
RAGI and RAG2 (rag) [Mombaerts P et al. Cell 1992; 68:869-77; Shinkai U et al. Cell 1992; 68:855-67];
NOD-scid [Greiner DL et al. 1998; Shultz L D et al. J Immunol 1995; 154:180-91; Melkus M W et al. Nature Med 2006; 12:1316-22; and Denton P W et al. PLOS Med 2008; 4 (12): e357];
IL2rg^{null} [DiSanto J P et al. Proc Natl Acad Sci USA 1995; 92:377-81];
B2m^{null} [Christianson S W et al. J Immunol 1997; 158:3578-86];
NSG® mice (NOD-scid IL2rγ^{null}) [Shultz L D et al. Nat Rev Immunol 2007; 7:118-30; Ito M et al. Blood 2002; 100:3175-82; Ishikawa I et al. Blood 2005; 106:1565-73; and Macchiarini F et al. i J Exp Med 2005; 202:1307-11];
NRG mice (NOD.Cg-Ragl^{tmlMom} Il2rg^{tmlWjl}/SzJ) [Pearson T et al. Clin Exp Immunol 2008 November; 154 (2): 270-84]
NOG mice (NOD.cg-Prkdc^{scid} Il2rg^{tmlSug}) [Shultz L D et al. Nat Rev Immunol 2007; 7:118-30];
NCG mice (NOD-Prkdc^{em26Cd52} Il2rg^{em26Cd22}/NjuCrl);
NOD-scid B2mnull [Shultz et al. 2007; Shultz L D et al. Transplantation 2003; 76:1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90 (9): 3913-7; and Roy C J et al. Infect Immun 2005; 73 (4): 2452-60]. See, e.g., Belizario J E The Open Immunology Journal, 2009; 2:79-85;
BRG mice (BALB/cA-Rag2^{null}Il2ry^{null}) [Goldman J P et al. Br J Haematol. 1998;103:335-342]; and
MISTRG mice (C;129S4-Rag2^{tml.lFlv} Csfl^{tml(CSF1)Flv} Csf2/Il3^{tml.l(CSF2,IL3)Flv} Thpo^{tml.l(TPO)/Flv} Il2rg^{tml.lFlv} Tg (SIRPA) 1Flv/J) [Rongvaux A et al. Nat Biotechnol. 2014 April;32 (4): 364-72].

Provided herein, in some embodiments, are immunodeficient mouse models having the non-obese diabetic (NOD) mouse genotype. The NOD mouse (e.g., Jackson Labs Stock #001976, NOD-ShiLtJ) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background has a genetic background (“background”) selected from NOD-Cg.-Prkdc^{scid} IL2rg^{tmlwJl}/SzJ (NSG®), a NOD.Cg-Ragl^{tmlMom} Il2rg^{tmlWjl}/SzJ (NRG), NOD.Cg-Prkdc^{scid}Il2rg^{tmlSug}/ShiJic (NOG), and NOD-Prkdc^{em26Ca52}Il2rg^{em26Cd22}/NjuCrl (NCG). Other immunodeficient mouse strains are contemplated herein.

In some embodiments, an immunodeficient mouse model based on the NOD background has an NOD-Cg.-Prkdc^{scid}Il2rg^{tmlwJl}/SzJ (NSG®) genetic background. The NSG® mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG® mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rgm^{tmlWjl} targeted mutation. The Il2rg^{tmlWjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The extreme immunodeficiency of NRG allows the mice to be humanized by engraftment of human CD34⁺ hematopoietic stem cells (HSC), human peripheral blood mononuclear cells, (PBMCs), and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-γ chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The

NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., α chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344, the contents of which are incorporated by reference herein.

An NSG-SGM3 mouse is the NSG derivative mouse NOD.Cg-Prkdc^{scid} Il2rg^{tmlWjl} Tg (CMV-IL3,CSF2,KITLG) 1Eav/MloySzJ (Jackson Laboratory Stock No: 013062). The transgenic NSG-SGM3 mice express three human cytokines: human Interleukin-3 (IL-3), human Granulocyte/Macrophage-colony stimulating factor 2 (GM-CSF), and human Stem Cell Factor (SCF). NSG-SGM3 mice combine the features of the highly immunodeficient NSG mouse with expression of human cytokines IL-3, GM-CSF, and SCF that support stable engraftment of myeloid lineages (e.g., monocytes, dendritic cells) and regulatory T cell populations.

In some embodiments, an NSG® mouse transgenically expresses human IL15. An NSG-IL-15 mouse, NOD.Cg-Prkdc^{scid} Il2rg^{tmlWjl} Tg (IL15) 1Sz/SzJ (Jackson Laboratory Stock No: 030890), expresses human IL15 and is combined with the highly immunodeficient NOD scid gamma (NSG) mouse. Expression of human IL15, in some embodiments, enhances the development of human NK cells in immunodeficient mice engrafted with human stem cells.

In some embodiments, an NSG-SGM3 mouse transgenically expresses human IL15 (i.e., NSG-SGM3 (hu-IL-15)).

Assessing an impaired immune system in mice can involve various methods and techniques, often depending on the specific type of immune impairment suspected or the specific research question at hand. Non-limiting examples of such methods include complete blood count (CBC), flow cytometry, cytokine analysis, immune challenge, histology and immunohistochemistry, T-cell proliferation assays, and serum antibody level analysis. CBC is a basic test that measures different components of the blood, including red blood cells, white blood cells, and platelets. Changes in these counts can indicate an immune response or immune deficiency. Flow cytometry can be used to analyze specific populations of immune cells in the blood, spleen, lymph nodes, or other tissues. By using antibodies tagged with fluorescent markers that bind to specific proteins on the surface of immune cells (known as cell markers or cluster of differentiation (CD) markers), researchers can identify and quantify different types of immune cells (e.g., T cells, B cells, macrophages, neutrophils). By measuring the levels of specific cytokines in the blood or tissues (using techniques like ELISA or multiplex assays), one can gain insights into the immune response. In immune challenge tests, mice are exposed to a specific antigen or pathogen, and the immune response is measured. This could involve measuring the response to a vaccine, the ability to clear a bacterial or viral infection, or the reaction to an inflammatory stimulus. Tissue samples from the mouse can be examined under a microscope to look for signs of inflammation or immune cell infiltration. With immunohistochemistry, specific types of immune cells can be labeled and visualized. T-cell proliferation assays measure the ability of T cells to proliferate in response to stimulation, which can be a key indicator of immune function. Measurement of specific antibodies in response to an antigen can indicate the functionality of the humoral immune response.

Assessing cell signaling deficiencies typically involves a combination of molecular biology techniques, cellular assays, and sometimes in vivo animal studies, for example. Non-limiting examples of methods for assessing cell signaling deficiencies include Western blot, flow cytometry, immunofluorescence, quantitative PCR (qPCR), reporter gene assays, RNA sequencing (RNA-Seq), protein arrays and mass spectrometry. Western blotting is commonly used to measure the levels of specific proteins, including those involved in cell signaling. It can be used to detect the presence of a signaling protein and to assess changes in protein level or modifications (like phosphorylation) that may indicate activation or inhibition of a signaling pathway. Flow cytometry can be used to assess cell surface markers or intracellular signaling proteins, enabling the identification and quantification of cell populations with specific signaling characteristics. It can also be used to measure changes in signaling proteins following stimulation or inhibition of cells. Immunofluorescence can be used to visualize the location and expression level of signaling proteins within cells using fluorescently labeled antibodies. Quantitative PCR can be used to measure changes in gene expression levels, which can be indicative of changes in cell signaling pathways. This can be particularly useful when studying transcription factors or other signaling molecules that regulate gene expression. Reporter gene assays involve engineering cells to express a reporter gene (like luciferase or green fluorescent protein) under the control of a promoter that is responsive to a specific signaling pathway. Activation or inhibition of the pathway can then be assessed by measuring the output of the reporter gene. RNA Sequencing can be used to look at global changes in gene expression, which can provide a broad view of the activation or inhibition of cell signaling pathways. Protein arrays and mass spectrometry can be used to look at changes in protein-protein interactions or post-translational modifications that can indicate changes in cell signaling.

Human FLT3L Transgene and/or Mouse Flt3 Inactivation

Some aspects provide an immunodeficient mouse that comprises a human FLT3L transgene (e.g., integrated into the genome of the mouse) or otherwise expresses or has been administered human FLT3L protein. Human FLT3L encodes FMS-related receptor tyrosine kinase 3 ligand (FLT3L), which controls the development of dendritic cells (DCs) and is particularly important for plasmacytoid DCs and CD8-positive classical DCs and their CD103-positive tissue counterparts. In some embodiments, an immunodeficient mouse expresses human FLT3L protein. In some embodiments, human FLT3L is expressed at a level of at least 5,000 pg/ml or at least 10,000 pg/ml. For example, human FLT3L may be expressed at a level of at least 5,000 pg/ml, 7,500 pg/ml, 10,000 pg/ml, 12,500 pg/ml, 15,000 pg/ml, 17,500 pg/ml, 20,000 pg/ml, 22,500 pg/ml, 25,000 pg/ml, 27,500 pg/ml, 30,000 pg/ml, 32,500 pg/ml, 35,000 pg/ml, 37,500 pg/ml, 40,000 pg/ml, 42,500 pg/ml, 45,000 pg/ml, 47,500 pg/ml, or 50,000 pg/ml. In some embodiments, human FLT3L is expressed at a level of 10,000 pg/ml to 30,000 pg/ml. In some embodiments, human FLT3L is expressed at a level of 15,000+/−1000 pg/mL to 17,000+/−100 pg/ml. Methods of detecting FLT3L protein expression are known and may be used as provided herein. For example, flow cytometry and/or an ELISA (enzyme-linked immunosorbent assay) using an anti-FLT3L antibody may be used to detect the level of human FLT3L protein present in mouse tissue and/or blood. A nonlimiting example of an immunodeficient mouse comprising a human FLT3L transgene is described in International Publication No. WO 2020/168029.

In some embodiments, an immunodeficient mouse may also comprise an endogenous Flt3L allele. Thus, in some embodiments, an immunodeficient mouse may also express endogenous mouse FLT3L protein. In some embodiments, mouse FLT3L is expressed at a level of at least 1,000 pg/ml or at least 2,000 pg/ml. For example, mouse FLT3L may be expressed at a level of 3,000 pg/ml, 4,000 pg/ml, 5,000 pg/ml, 6,000 pg/ml, 7,000 pg/ml, 8,000 pg/ml, 9,000 pg/ml, or 10,000 pg/ml. In some embodiments, mouse FLT3L is expressed at a level of 5,000 pg/ml to 10,000 pg/ml. In some embodiments, mouse FLT3L is expressed at a level of 6,000 pg/ml to 8,000 ml.

In some embodiments, an endogenous Flt3 allele is inactivated in the immunodeficient mouse, for example, as described in International Publication No. WO 2020/168029. Thus, in some embodiments, a method comprises administering cells from a T cell-negative fraction to an immunodeficient mouse, wherein the immunodeficient mouse has been engrafted with diseased human cells, has a null mutation in one or more Flt3 alleles (e.g., does not express endogenous Flt3), and expresses human FLT3L. In other embodiments, a method comprises administering diseased human cells to an immunodeficient mouse, and administering cells from a T cell-negative fraction to the immunodeficient mouse, wherein the immunodeficient mouse has a null mutation in one or more Flt3 alleles (e.g., does not express endogenous Flt3), and expresses human FLT3L.

In some embodiments, a human FLT3L protein is delivered to an immunodeficient mouse, for example, by tail vein injection. Thus, in some embodiments, a human FLT3L protein is not expressed by the genome of the immune deficient mouse, but rather is delivered as a protein or is delivered via a vector encoding the protein (e.g., an extrachromosomal

### Myeloablation

In some embodiments, immunodeficient mice are treated to deplete and/or suppress any remaining murine immune cells (e.g., chemically and/or with radiation). In some embodiments, immunodeficient mice are treated only chemically or only with radiation. In other embodiments, immunodeficient mice are treated both chemically and with radiation.

In some embodiments, immunodeficient mice are administered a myeloablative agent, that is, a chemical agent that suppresses or depletes murine immune cells. Examples of myeloablative agents include busulfan, treosulfan, dimethyl mileran, melphalan, and thiotepa.

In some embodiments, immunodeficient mice are irradiated prior to engraftment with human cells,, such as human T cell-negative PBMC fractions. It is thought that irradiation of an immunodeficient mouse destroys mouse immune cells in peripheral blood, spleen, and bone marrow, which facilitates engraftment of human cells, such as human PBMCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG® mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 125, 150, 175, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy.

### Humanization

In some embodiments, the immunodeficient mice are engrafted with a T cell-negative fraction or PBMCs obtained from a T cell-negative fraction, thereby humanizing the mice.

Engraftment refers to the process of the human cells (e.g., cells of a T cell-negative fraction) migrating to, and incorporating into, an existing tissue of interest in vivo. The human cells may be engrafted after irradiation and before engraftment of other human cells (e.g., human diseased cells), after irradiation and concurrently with engraftment of other human cells, or after irradiation and after engraftment of other human cells.

Peripheral blood mononuclear cells are a type of white blood cell that is found in the bloodstream and have a round nucleus. These mononuclear blood cells recirculate between tissues and blood and are a critical component in the immune system to fight infection and adapt to intruders. There are two main types of PBMCs: lymphocytes and monocytes. The majority (˜70-90%) of an enriched human PBMC sample is composed of lymphocytes (white blood cells), which include CD4+ helper T cells, CD8+ killer T cells, B cells, and Natural Killer (NK) cells. Monocytes make up a smaller portion (˜10-30%) of the enriched human PBMC sample. Monocytes, when stimulated, can differentiate into macrophages or dendritic cells.

Methods of engrafting immunodeficient mice with human cells to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32 (2): 194-2020; Yaguchi et al., Cell & Mol Immunol, 2018, 15:953-962).

In some embodiments, an immunodeficient mouse is administered (e.g., engrafted with) 0.5×106-50x106 human cells (e.g., from a T cell-negative fraction). For example, an immunodeficient mouse may be administered (e.g., engrafted with) 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human cells (e.g., from a T cell-negative fraction). In some embodiments, an immunodeficient mouse is administered (e.g., engrafted with) about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human cells (e.g., from a T cell-negative fraction).

In some embodiments, an immunodeficient mouse is administered (e.g., engrafted with) human cells (e.g., from a T cell-negative fraction), for example, following a myeloablative treatment, such as sublethal irradiation or chemical ablation. In some embodiments, an immunodeficient mouse is administered (e.g., engrafted with) human cells (e.g., from a T cell-negative fraction) about 15 minutes, 30 minutes, 45 minutes, 1 hour, or more following the myeloablative treatment. In some embodiments, an immunodeficient mouse is administered (e.g., engrafted with) human cells (e.g., from a T cell-negative fraction) about 1 to 5 days, about 1 to 10 days, or about 1 to 20 days, e.g., about 1, 2, 3, 4, 5,6, 7, 8, 9, 10, 11, 12, 13, 14, 15, 16, 17, 18, 19, or 20 days following the myeloablative treatment.

Engrafted human cells may express and/or secrete cell signaling molecules, such as cytokines, for example. Cytokines are a broad category of small proteins that are important in cell signaling. They are released by cells and affect the behavior of other cells. Cytokines include chemokines, interferons, interleukins, lymphokines, and tumor necrosis factors, among others. They're produced by a broad range of cells, including immune cells like macrophages, B lymphocytes, T lymphocytes and mast cells, as well as endothelial cells, fibroblasts, and various stromal cells. Cytokines play a crucial role in the body's response to disease and infection, as well as in the regulation of immune responses, inflammation, and the formation of blood cells (hematopoiesis). They can be either pro-inflammatory (promoting inflammation) or anti-inflammatory (reducing inflammation). Non-limiting example of human serum cytokines include interleukins (ILs), interferons (IFNs), tumor necrosis factors (TNFs), growth factors, and chemokines. Interleukins are a group of cytokines that were first seen to be expressed by white blood cells (leukocytes). Examples include IL-1, IL-2, IL-4, IL-6, IL-10, IL-12, IL-17, etc. Each has specific effects on the interactions and communications between cells. Interferons are a group of signaling proteins that are released by host cells in response to the presence of several pathogens, such as viruses, bacteria, parasites, and also tumor cells. Examples include IFN-alpha, IFN-beta, and IFN-gamma. Tumor necrosis factors are a group of cytokines that can cause cell death (apoptosis). An example is TNF-alpha, which plays a role in systemic inflammation and is involved in the acute phase reaction. Growth Factors are proteins that stimulate cell growth, proliferation, and differentiation. Examples include Epidermal Growth Factor (EGF), Fibroblast Growth Factor (FGF), and Vascular Endothelial Growth Factor (VEGF). Chemokines are a family of small cytokines, or signaling proteins secreted by cells. They induce chemotaxis in nearby responsive cells, guiding the migration of cells. Examples include CXCL8 (IL-8), CCL2 (MCP-1), and CXCL10 (IP-10).

### Diseased Cell Engraftment

In some embodiments, a human cell is a diseased cell. There are numerous cell types that can become diseased in the human body. For instance, cancerous cells are a type of diseased cell that divide uncontrollably and can invade nearby tissues. Other examples include infected cells such as those infected with viruses, bacteria, or fungi, which can cause diseases such as influenza, tuberculosis, or meningitis. In autoimmune diseases such as rheumatoid arthritis, the body's immune system mistakenly attacks healthy cells, leading to inflammation and tissue damage. Additionally, in genetic disorders like sickle cell anemia, red blood cells become misshapen and unable to carry oxygen effectively, leading to a host of health problems. Finally, in neurological diseases such as Alzheimer's, neurons in the brain become damaged and die, leading to cognitive decline and memory loss.

In some embodiments, the diseased human cells are cancerous cells. Cancerous cells are cells that divide uncontrollably and can invade nearby tissues. In some embodiments, the diseased human cells are infected cells. Infected cells are cells that are infected with viruses, bacteria, fungi, or parasites that cause diseases such as influenza, tuberculosis, meningitis, and HIV. In some embodiments, the diseased human cells are autoimmune cells.

Autoimmune cells are cells in the immune system that mistakenly attack healthy cells and tissues, leading to autoimmune diseases such as rheumatoid arthritis, multiple sclerosis, and lupus. In some embodiments, the diseased human cells are red blood cells. Red blood cells are cells in the blood that carry oxygen throughout the body, which can be affected in conditions such as sickle cell anemia, hemophilia, and thalassemia. In some embodiments, the diseased human cells are white blood cells. White blood cells are cells in the immune system that help fight infections and diseases, which can be affected in conditions such as leukemia, lymphoma, and myeloma. In some embodiments, the diseased human cells are platelets. Platelets are cells in the blood that help with blood clotting, which can be affected in conditions such as thrombocytopenia and hemophilia. In some embodiments, the diseased human cells are neurons. Neurons are cells in the brain and nervous system that transmit information, which can be affected in conditions such as Alzheimer's, Parkinson's, and multiple sclerosis. In some embodiments, the diseased human cells are cardiac cells. Cardiac cells are cells in the heart that control its function, which can be affected in conditions such as heart failure, arrhythmia, and cardiomyopathy. In some embodiments, the diseased human cells are lung cells. Lung cells are cells in the lungs that help with breathing, which can be affected in conditions such as asthma, chronic obstructive pulmonary disease (COPD), and lung cancer. In some embodiments, the diseased human cells are liver cells. Liver cells are cells in the liver that help with detoxification and metabolism, which can be affected in conditions such as hepatitis, cirrhosis, and liver cancer.

There are many different types of cancer, each of which originates from different types of cells in the body. Non-limiting examples of cancerous (cancer) cells include breast cancer, lung cancer, prostate cancer, colorectal cancer, skin cancer, stomach cancer, leukemia, lymphoma, brain cancer, pancreatic cancer, and ovarian cancer cells.

In some embodiments, the diseased human cells are immortalized cells, i.e., from a cell line. Immortalized cells are cells that has been manipulated, either through genetic modification or via exposure to specific viruses or chemicals, to proliferate indefinitely. Thus, they don't undergo the normal process of senescence (cellular aging) and cell death (apoptosis) that would naturally limit their lifespan. Non-limiting examples of immortalized cell lines include Hela cells, MCF-7 cells, PC-3 cells, A549 cells, HepG2 cells, Jurkat cells, and U87 cells.

In some embodiments, a patient-derived xenograft (PDX) or cells of a PDX are delivered to a mouse model provided herein. A PDX is a cancerous tissue from a patient that is implanted into an immunodeficient mouse. A PDX is used, in some embodiments, to maintain the heterogeneity and architecture of the original tumor, enabling researchers to study cancer in a more natural and clinically relevant environment compared to traditional in vitro methods. Because PDX models preserve the complexity of the cancer, including its unique genetics and the interactions between the cancer cells and their microenvironment, they can provide more accurate predictions about how the tumor may respond to various therapies.

The number of diseased human cells administered to an immunodeficient mouse to establish a tumor, for example, can vary widely depending on the type of cells, the mouse strain, and the specific experimental design. In some embodiments, 10\hat{ }5 to 10\hat{ }7 (100,000 to 10,000,000) cells are administered to the mouse. In other embodiments, a lower number of cells may be administered to see if they can establish a tumor, and then an increased number of cells may be administered if needed. In other embodiments, for example, if the cancer cells are very aggressive and form tumors easily, fewer cells are administered. The method of injection can influence the number of cells needed. For example, cells injected directly into the tissue (orthotopic injection) might establish a tumor with fewer cells compared to cells injected into the bloodstream (intravenous injection) or under the skin (subcutaneous injection).

Diseased cells, in some embodiments, are administered systemically, as described elsewhere herein. When cells are introduced into a mouse via intravenous injection through the tail vein, they often first accumulate in the lungs. This happens because the pulmonary capillary system acts as a filter for the cells, trapping them in the lungs due to the narrow and complex network of blood vessels. Whether these cells engraft and integrate into the lung tissue depends on several factors including the type of cell, the status of the recipient mouse (e.g., any pre-conditioning like irradiation or chemotherapy), and whether the injected cells have the appropriate receptors for adhesion and interaction with lung tissue. The use of intravenous tail vein injection, for example, in mice is a common method for assessing the metastatic potential of cancer cells. This is because cancer cells often have the ability to invade, migrate, and establish new tumors (metastases) in distant organs like the lungs.

### Cells and Cell Lines

In some embodiments, the immunodeficient mice are engrafted with primary cells. Human primary cells are isolated directly from tissues and retain the morphological and functional characteristics of their tissue of origin. In some embodiments, a primary cell is a cancer cell. In some embodiments, a primary cell is a neuronal cell. In some embodiments, a primary cell is a metabolic cell. In some embodiments, a primary cell is a cardiac cell. Other primary cells are contemplated herein.

In some embodiments, the immunodeficient mice are engrafted with stem cells, such as induced pluripotent stem cells (iPSCs). iPSCs are a type of pluripotent stem cell that can be generated directly from a somatic cell (see, e.g., Takahashi K et al. Cell. 2006; 126 (4): 663-76).

In some embodiments, the immunodeficient mice are engrafted with immortalized cells (immortalized cell lines). Immortalized cell lines are cells that have been manipulated to proliferate indefinitely and can thus be cultured for long periods of time. Non-limiting examples of commonly used immortalized cell lines include 3T3 cells, HeLa cells, COS cells, 293/293T/HEK-293T cells, MDCK cells, CHO cells, S2 cells, PC12 cells, Neuro-2a/N2a cells, and SH-SY5Y cells. Other immortalized cells are contemplated herein and described below.

In some embodiments, the immunodeficient mice are engrafted with tumor cells from tumor cell lines. Tumor cell lines are known in the art and are publicly accessible, for example, through ATCC or other collections. In some embodiments, the cell line is from a human tumor. In some embodiments, Raji, a cell line associated with human B cell lymphoma is used. In some embodiments, Jeko-1, a cell line associated with human mantle cell lymphoma is used. Examples of tumor cell lines include, but are not limited to, human lung carcinoma cell lines, such as A549 (SRCC768), Calu-1 (SRCC769), Calu-6 (SRCC770), H157 (SRCC771), H441 (SRCC772), H460 (SRCC773), SKMES-1 (SRCC774), SW900 (SRCC775), H522 (SRCC832), and H810 (SRCC833).

In some embodiments, the cell line is associated with human lung tumors, such as SRCC724 (adenocarcinoma, abbreviated as “AdenoCa”) (LT1), SRCC725 (squamous cell carcinoma, abbreviated as “SqCCa) (LTla), SRCC726 (adenocarcinoma) (LT2), SRCC727 (adenocarcinoma) (LT3), SRCC728 (adenocarcinoma) (LT4), SRCC729 (squamous cell carcinoma) (LT6), SRCC730 (adeno/squamous cell carcinoma) (LT7), SRCC731 (adenocarcinoma) (LT9), SRCC732 (squamous cell carcinoma) (LT10), SRCC733 (squamous cell carcinoma) (LT11), SRCC734 (adenocarcinoma) (LT12), SRCC735 (adeno/squamous cell carcinoma) (LT13), SRCC736 (squamous cell carcinoma) (LT15), SRCC737 (squamous cell carcinoma) (LT16), SRCC738 (squamous cell carcinoma) (LT 17), SRCC739 (squamous cell carcinoma) (LT18), SRCC740 (squamous cell carcinoma) (LT19), SRCC741 (lung cell carcinoma, abbreviated as “LCCa”) (LT21), SRCC811 (adenocarcinoma) (LT22), SRCC825 (adenocarcinoma) (LT8), SRCC886 (adenocarcinoma) (LT25), SRCC887 (squamous cell carcinoma) (LT26), SRCC888 (adeno-BAC carcinoma) (LT27), SRCC889 (squamous cell carcinoma) (LT28), SRCC890 (squamous cell carcinoma) (LT29), SRCC891 (adenocarcinoma) (LT30), SRCC892 (squamous cell carcinoma) (LT31), SRCC894 (adenocarcinoma) (LT33). Also included are human lung tumors designated SRCC1125 [HF-000631], SRCC1127 [HF-000641], SRCC1129 [HF-000643], SRCC1133 [HF-000840], SRCC1135 [HF-000842], SRCC1227 [HF-001291], SRCC1229 [HF-001293], SRCC1230 [HF-001294], SRCC1231 [HF-001295], SRCC1232 [HF-001296], SRCC1233 [HF-001297], SRCC1235 [HF-001299], and SRCC1236 [HF-001300].

In some embodiments, the cell line is associated with human colon cancers. Examples of colon cancer cell lines include, but are not limited to, SW480 (adenocarcinoma, SRCC776), SW620 (lymph node metastasis of colon adenocarcinoma, SRCC777), Colo320 (carcinoma, SRCC778), HT29 (adenocarcinoma, SRCC779), HM7 (a high mucin producing variant of ATCC colon adenocarcinoma cell line, SRCC780), CaWiDr (adenocarcinoma, SRCC781), HCT116 (carcinoma, SRCC782), SKCO1 (adenocarcinoma, SRCC783), SW403 (adenocarcinoma, SRCC784), LS174T (carcinoma, SRCC785), Colo205 (carcinoma, SRCC828), HCT15 (carcinoma, SRCC829), HCC2998 (carcinoma, SRCC830), and KM12 (carcinoma, SRCC831). Primary colon tumors include colon adenocarcinomas designated

CT2 (SRCC742), CT3 (SRCC743), CT8 (SRCC744), CT10 (SRCC745), CT12 (SRCC746), CT14 (SRCC747), CT15 (SRCC748), CT16 (SRCC749), CT17 (SRCC750), CT1 (SRCC751), CT4 (SRCC752), CT5 (SRCC753), CT6 (SRCC754), CT7 (SRCC755), CT9 (SRCC756), CT11 (SRCC757), CT18 (SRCC758), CT19 (adenocarcinoma, SRCC906), CT20 (adenocarcinoma, SRCC907), CT21 (adenocarcinoma, SRCC908), CT22 (adenocarcinoma, SRCC909), CT23 (adenocarcinoma, SRCC910), CT24 (adenocarcinoma, SRCC911), CT25 (adenocarcinoma, SRCC912), CT26 (adenocarcinoma, SRCC913), CT27 (adenocarcinoma, SRCC914),CT28 (adenocarcinoma, SRCC915), CT29 (adenocarcinoma, SRCC916), CT30 (adenocarcinoma, SRCC917), CT31 (adenocarcinoma, SRCC918), CT32 (adenocarcinoma, SRCC919), CT33 (adenocarcinoma, SRCC920), CT35 (adenocarcinoma, SRCC921), and CT36 (adenocarcinoma, SRCC922). Also included are human colon tumors designated SRCC1051 [HF-000499], SRCC1052 [HF-000539], SRCC1053 [HF-000575], SRCC1054 [HF-000698], SRCC1142 [HF-000762], SRCC1144 [HF-000789], SRCC1146 [HF-000795] and SRCC1148 [HF-000811].

In some embodiments, the cell line is associated with human breast cancers. Examples of human breast carcinoma cell lines include, for example, HBL100 (SRCC759), MB435s (SRCC760), T47D (SRCC761), MB468 (SRCC762), MB175 (SRCC763), MB361 (SRCC764), BT20 (SRCC765), MCF7 (SRCC766), and SKBR3 (SRCC767), and human breast tumor center designated SRCC1057 [HF-000545]. Also included are human breast tumors designated SRCC1094, SRCC1095, SRCC1096, SRCC1097, SRCC1098, SRCC1099, SRCC1100, SRCC1101, and human breast-met-lung-NS tumor designated SRCC893 [LT 32].

In some embodiments, the cell line is associated with human kidney cancers. Examples of human kidney tumor cell lines include SRCC989 [HF-000611] and SRCC1014 [HF-000613].

In some embodiments, the cell line is associated with human testicular cancers. Examples of human testis tumor center includes SRCC1001 [HF-000733] and testis tumor margin SRCC999 [HF-000716].

In some embodiments, the cell line is associated with human parathyroid cancers. Examples of human parathyroid tumor cell lines include SRCC1002 [HF-000831] and SRCC1003 [HF-000832].

Other human tumor cell lines are contemplated herein.

In some embodiments, the immunodeficient mice are injected with an appropriate amount of tumor cells from a cancer cell line. In some embodiments, the immunodeficient mice are injected with 10\hat{ }5 to 10\hat{ }7 (100,000 to 10,000,000) cells.

In some embodiments, the cancer cells are introduced into the mice before the PBMCs are engrafted and the human immune cells (e.g., human B cells or T cells or NK cells) appear. In some embodiments, the tumor cells are introduced immediately after myeloablation.

### Routes of Administration

Cells (e.g., human cells) and/or therapeutic modalities (or other substances/agents) of the present disclosure may be administered to an immunodeficient mouse via systemic administration or via local administration, for example.

In some embodiments, a cell or therapeutic modality is administered via systemic administration. Systemic routes of administration in mice involve the methods by which drugs or therapeutic modalities are introduced into the body of mice to achieve systemic distribution and desired effects. Intravenous (IV) injection is a commonly used route in mice, involving the direct delivery of drugs or therapeutic modalities into veins such as the tail vein, lateral tail vein, retro-orbital sinus, or jugular vein. IV injection provides rapid and direct access to the systemic circulation, ensuring immediate distribution throughout the body. This route is suitable for substances requiring quick systemic effects. Intraperitoneal (IP) injection involves the delivery of drugs or therapeutic modalities into the peritoneal cavity of mice. The substance is absorbed through the peritoneal membrane and enters the systemic circulation. This route provides widespread distribution of the drug within the abdominal cavity and systemic circulation, making it suitable for drugs requiring extensive contact with abdominal organs. Subcutaneous (SC) injection involves the delivery of drugs or therapeutic modalities into the subcutaneous tissue, typically in the dorsal region or behind the neck of mice. The substance is absorbed into the systemic circulation through the capillaries in the subcutaneous tissue. SC injection provides slower but sustained release of the drug into the systemic circulation, making it suitable for substances requiring a longer duration of action. Intramuscular (IM) injection entails the delivery of drugs or therapeutic modalities directly into the muscle tissue of mice, such as the quadriceps or gastrocnemius muscle. The substance is absorbed through the capillaries within the muscle and enters the systemic circulation. IM injection allows for sustained release and longer duration of action compared to other routes, making it suitable for substances requiring a sustained effect. Oral gavage involves the administration of drugs or therapeutic modalities directly into the stomach of mice using a feeding needle or oral gavage needle. This route is commonly used for substances that are orally bioavailable and stable in the gastrointestinal tract. Oral gavage allows for systemic distribution through absorption in the gastrointestinal tract, making it suitable for substances that can be administered orally. Inhalation involves the administration of drugs or therapeutic modalities through inhalation of aerosolized substances. Inhalation chambers or specialized devices are used to deliver the substance to the respiratory system of mice. Inhalation allows for targeted delivery to the lungs and systemic distribution through absorption in the respiratory tract. This route is suitable for substances targeting the respiratory system or requiring direct delivery to the lungs.

In some embodiments, a cell or therapeutic modality is administered via local administration. Local routes of administration in mice involve delivering drugs or therapeutic modalities directly to specific target tissues or regions of interest within the mouse body. These routes focus on localized delivery for localized effects, as opposed to systemic routes that aim for widespread distribution. Various local routes of administration are commonly used in mice for specific research objectives. Intradermal (ID) injection is a local route that delivers drugs or therapeutic modalities into the dermis, the layer of skin directly beneath the epidermis. This route is suitable for substances targeting the skin or requiring localized effects in the skin tissue. Subcutaneous (SC) injection, traditionally associated with systemic administration, can also be employed for local administration in mice. By targeting specific subcutaneous regions or anatomical sites, drugs or therapeutic modalities can be delivered directly to the desired local area. Intramuscular (IM) injection serves as both a systemic and local route of administration. In the context of local administration, the drug or therapeutic agent is injected directly into the muscle tissue at the specific site of interest. Intraperitoneal (IP) injection, primarily considered a systemic route, can also be utilized for local administration within the abdominal cavity. By delivering the drug or therapeutic agent into the peritoneal cavity, localized effects can be achieved in organs or tissues within the abdominal region. Intra-articular injection involves delivering drugs or therapeutic modalities directly into the joint space. Intranasal administration entails delivering drugs or therapeutic modalities through the nasal cavity. This local route allows for targeted effects in the nasal passages or the potential to target the central nervous system through the olfactory route. Topical administration involves the application of drugs or therapeutic modalities directly onto the skin or mucous membranes. This local route allows for localized effects on the skin or mucosal surfaces, such as the eyes, ears, or genitals.

In some embodiments, cells and/or agents are administered orthotopically. Orthotopic administration refers to the delivery of drugs or therapeutic modalities directly to the anatomically correct or appropriate location within an organism, mimicking the natural or original site of the disease or condition being studied. In the context of animal research, particularly in mice, orthotopic administration aims to reproduce the physiological and anatomical characteristics of a specific organ or tissue to study disease progression, treatment response, or other relevant biological processes. Orthotopic administration in mice involves various techniques to target specific organs or tissues. One commonly used approach is orthotopic tumor implantation, where tumor cells or tissues are injected or surgically placed directly into the corresponding anatomical site of interest. This method allows researchers to study tumor growth, metastasis, and treatment response in a manner that closely resembles the natural environment of the tumor. Another approach is organ-specific injection, where drugs or therapeutic modalities are delivered directly into a specific organ or tissue of interest. By injecting cells or other substances into organs like the liver, lungs, brain, or other organs, researchers can investigate organ-specific effects, disease models, or therapeutic interventions. Orthotopic transplantation is another technique used in mice, involving the surgical transfer or transplantation of tissues or cells to their anatomically correct location within the recipient mouse. This method is commonly used in transplantation studies to assess graft survival, integration, and functionality. Orthotopic infusion or instillation involves the direct introduction of substances into organs or cavities through a catheter or needle. For example, instilling drugs into the bladder or bronchi can mimic the physiological conditions of urinary or respiratory diseases, enabling researchers to study localized effects or treatment approaches.

In some embodiments, cells of the disclosure (e.g., human cells) are administered to a mammary fat pad of an immunodeficient mouse. The mammary fat pad of a mouse model refers to a specialized region of adipose (fat) tissue located within the mammary gland area of female mice. In female mice, the mammary glands are situated in pairs along the abdominal region. Each mammary gland is composed of multiple lobes and ductal structures embedded within the surrounding mammary fat pad.

In some embodiments, cells of the disclosure (e.g., human cells) are administered to a renal capsule of an immunodeficient mouse. The renal capsule of a mouse refers to the outer layer or covering that encapsulates the kidneys. It is a fibrous layer composed of connective tissue that surrounds and protects the kidneys, providing structural support. The renal capsule acts as a barrier, separating the kidneys from the surrounding tissues and organs. The renal capsule is often utilized for various procedures, including transplantation or implantation of cells, tissues, or therapeutic modalities into the kidney. This can involve making an incision in the renal capsule to access the kidney and perform the desired manipulation.

### Methods for Assessing Efficacy of a Therapeutic Agent

In some embodiments, the methods provided herein are for assessing the efficacy of a therapeutic agent. The terms “therapeutic agent” and “therapeutic modality” are used interchangeably herein. Non-limiting examples of therapeutic agents include engineered immune cell (e.g., T cell, B cell, or NK cell) therapies, antibody and/or other recombinant protein therapies, nucleic acid therapies (e.g., mRNA, antisense, and/or RNAi), and small molecule drug (e.g., less than 1000 g/mol or less than 1 kDa) therapies.

In some embodiments, a humanized mouse disease model can be established by engrafting a myeloablated (e.g., an irradiated) immunodeficient mouse with diseased cells. After the diseased cells have grown for a sufficient amount of time in vivo, the mouse is treated with the prospective therapeutic agent. In some embodiments, a humanized mouse cancer model can be established by engrafting an irradiated immunodeficient mouse with tumor cells (e.g., cells from a tumor cell line or a PDX). After the tumor has grown for a sufficient time in vivo, the mouse is treated with the prospective therapeutic agent. By “sufficient time” it is meant that the tumor is the size or has the number of tumor cells needed to study the effects of the therapeutic agent, or in the case of a non-cancer disease mouse model, “sufficient time” refers to the amount of time needed to obtain the number of diseased cells necessary to study the effects of the therapeutic agent.

The human cells (e.g., PBMCs) from a T cell negative fraction may be administered before or after the diseased cells are administered. In some embodiments, the diseased cells are administered, then the human cells (e.g., PBMCs) from a T cell negative fraction are administered (e.g., up to 5 days later). In other embodiments, the human cells (e.g., PBMCs) from a T cell negative fraction are administered, then the diseased cells are administered (e.g., up to 5 days later). In some embodiments, the diseased cells and the human cells (e.g., PBMCs) from a T cell negative fraction are administered at the same time (e.g., within a few minutes or hours of each other).

With respect to cancer mouse models, the tumor cells can be grown in the mouse to establish varying tumor burdens (e.g., the longer the mouse is untreated, the higher the tumor burden). As used herein, “tumor burden” refers to the total number of cancer cells or the total size of the tumor(s) in the model mouse. Tumor burdens can be low, moderate, or high and may be expressed as a percentage of the total body weight of the mouse. High tumor burdens are >5% of the body weight of the mouse (e.g., 5%, 5.1%, 5.2%, 5.3%, 5.4%, 5.5%, 5.6%, 5.7%, 5.8%, 5.9%, 6% or more). In some embodiments, the high tumor burden is 5-5.2%, 5-5.3%, 5-5.4%, 5-5.5%, 5-5.6%, 5-5/8%, 5-5.8%,5-5.9%, 5-2%, 5.5-5.2%, 5.5-5.3%, 5.5-5.4%, 5.5-5.5%, 5.5-5.6%, 5.5-5.7%, 5.5-5.8%, 5.5-5.9%, 5.5-2%, 5.2-5.3%, 5.2-5.4%, 5.2-5.5%, 5.2-5.6%, 5.2-5.7%, 5.2-5.8%, 5.2-5.9%, 5.2-2%, 5.3-5.4%, 5.3-5.5%, 5.3-5.6%, 5.3-5.7%, 5.3-5.8%, 5.3-5.9%, 5.3-2%, 5.4-5.5%, 5.4-5.6%, 5.4-5.7%, 5.4-5.8%, 5.4-5.9%, 5.4-2%, 5.5-5.6%, 5.5-5.7%, 5.5-5.8%, 5.5-5.9%, 5.5-6%, 5.6-5.7%, 5.6-5.8%, 5.6-5.9%, 5.6-2%, 5.7-5.8%, 5.7-5.9%, 5.7-2%, 5.8-5.9%, 5.8-2%, or 5.9-6%. Moderate tumor burdens are 1<5% of the body weight of the mouse (e.g., 1%, 1.25%, 1.5%, 1.6%, 1.7%, 1.8%, 1.9%, 1.95%, 1.96%, 1.97%, 1.98%, 1.99%, 2%, 2.25%, 2.5%, 2.75%, 3%, 3.25%, 3.5%, 3.75%, 4%, 4.25% 4.5%, 4.75%). In some embodiments, the moderate tumor burden is 1.5-1.6%, 1.5-1.7%, 1.5-1.8%, 1.5-1.9%, 1.6-1.7%, 1.6-1.8%, 1.6-1.9%, 1.7-1.8%, 1.7-1.9%, or 1.8-1.9%. In some embodiments, the moderate tumor burden is 1-2%, 1-2.25%, 1-2.5%, 1-2.75%, 1-3%, 1-3.25%, 1-3.5%, 1-3.75%, 1-4%, 1-4.25%, 1-4.5%, 1-4.75%, 2-2.25%, 2-2.5%, 2-2.75%, 2-3%, 2-3.25%, 2-3.5%, 2-3.75%, 2-4%, 2-4.25%, 2-4.5%, 2-4.75%, 2.5-2.75%, 2.5-3%, 2.5-3.25%, 2.5-3.5%, 2.5-3.75%, 2.5-4%, 2.5-4.25%, 2.5-4.5%, 2.5-4.75%, 3-3.25%, 3-3.5%, 3-3.75%, 3-4%, 3-4.25%, 3-4.5%, 3-4.75%, 3.5-3.75%, 3.5-4%, 3.5-4.25%, 3.5-4.5%, 3.5-4.75%, 4-4.25%, 4-4.5%, 4-4.75%, or 4.5-4.75%. Low tumor burdens are less than 1% of the mouse's body weight (e.g., 0.05%, 0.1%, 0.2%, 0.3%, 0.4%, 0.45%, 0.46%, 0.47%, 0.48%, or 0.49%). In some embodiments, the low tumor burden is 0.1-0.2%, 0.1-0.3%, 0.1-0.4%, 0.2-0.3%, 0.2-0.4%, or 0.3-0.4%.

In some embodiments, the therapeutic agent is administered 1, 2, 3, 4, 5, 6, 7, 8, 9, 10, 11, 12, 13, or 14 days after the mouse was injected with the diseased cells (e.g., tumor cells) (e.g., 1-2 days, 1-3 days, 1-4 days, 1-5 days, 1-6 days, 1-7 days, 1-8 days, 1-9 days, 1-10 days, 1-11 days, 1-12 days, 1-13 days, 1-14 days, 2-3 days, 2-4 days, 2-5 days, 2-6 days, 2-7 days, 2-8 days, 2-9 days, 2-10 days, 2-11 days, 2-12 days, 2-13 days, 2-14 days, 3-4 days, 3-5 days, 3-6 days, 3-7 days, 3-8 days, 3-9 days, 3-10 days, 3-11 days, 3-12 days, 3-13 days, 3-14 days, 4-5 days, 4-6 days, 4-7 days, 4-8 days, 4-9 days, 4-10 days, 4-11 days, 4-12 days, 4-13 days, 4-14 days, 5-6 days, 5-7 days, 5-8 days, 5-9 days, 5-10 days, 5-11 days, 5-12 days, 5-13 days, 5-14 days, 6-7 days, 6-8 days, 6-9 days, 6-10 days, 6-11 days, 6-12 days, 6-13 days, 6-14 days, 7-8 days, 7-9 days, 7-10 days, 7-11 days, 7-12 days, 7-13 days, 7-14 days, 8-9 days, 8-10 days, 8-11 days, 8-12 days, 8-13 days, 8-14 days, 9-10 days, 9-11 days, 9-12 days, 9-13 days, 9-14 days, 11-12 days, 11-13 days, 11-14 days, 12-13 days, 12-14 days, or 13-14 days). Optionally, the human cells (e.g., PBMCs) from a T cell negative fraction may be administered with a therapeutic agent (e.g., in the same dose or in a different dose simultaneously).

In some embodiments, the therapeutic agent is an engineered immune cell (e.g., T cell, B cell, or NK cell) therapy and may be administered at a dose of 1-20×10⁶ engineered immune cells (e.g., T cells, B cells, or NK cells) cells, for example 1-2×10⁶, 1-3×10^{6, 1}-4×10^{6, 1}-5×10⁶, 1-6×10⁶, 1-7×10⁶, 1-8×10⁶, 1-9×10⁶, 1-10×10⁶, 1-12×10⁶, 1-14×10⁶, 1-16×10⁶, 1-18×10⁶, 1-20×10⁶, 2-3×10⁶, 2-4×10⁶, 2-5×10⁶, 2-6×10⁶, 2-7×10⁶, 2-8×10⁶, 2-9×10⁶, 2-10×10⁶, 2-12×10⁶, 2-14×10⁶, 2-16×10⁶, 2-18×10⁶, 2-20×10⁶, 3-4×10⁶, 3-5×10⁶, 3-6×10⁶, 3-7×10⁶, 3-8×10⁶, 3-9×10⁶, 3-10×10⁶, 3-12×10⁶, 3-14×10⁶, 3-16×10^{6, 3}-18×10⁶, 3-20×10⁶, 4-5×10⁶, 4-6×10⁶, 4-7×10⁶, 4-8×10⁶, 4-9×10⁶, 4-10×10⁶, 4-12×10⁶, 4-14×10⁶, 4-16×10⁶, 4-18×10⁶, 4-20×10⁶, 5-6×10^{6, 5}-7×10⁶, 5-8×10⁶, 5-9×10⁶, 5-10×10⁶, 5-12×10⁶, 5-14×10⁶, 5-16×10⁶, 5-18×10⁶, 5-20×10^{6, 6}-7×10⁶, 6-8×10⁶, 6-9×10⁶, 6-10×10⁶, 6-12×10⁶, 6-14×10⁶, 6-16×10⁶, 6-18×10⁶, 6-20×10⁶, 7-8×10⁶, 7-9×10⁶, 7-10×10⁶, 7-12×10⁶, 7-14×10⁶, 7-16×10⁶, 7-18×10⁶, 7-20×10⁶, 8-9×10⁶, 8-10×10⁶, 8-12×10⁶, 8-14×10⁶, 8-16×10⁶, 8-18×10⁶, 8-20×10⁶, 9-10×10⁶, 9-12×10⁶, 9-14×10⁶, 9-16×10⁶, 9-18×10⁶, 9-20×10⁶, 10-12×10⁶, 10-14×10⁶, 10-16×10⁶, 10-18×10⁶, 10-20×10⁶, 12-14×10⁶, 12-16×10⁶, 12-18×10⁶, 12-20×10⁶, 14-16×10⁶, 14-18×10⁶, 14-20×10⁶, 16-18×10⁶, 16-20×10⁶, 18-20×10⁶ engineered immune cells. In some embodiments, the mouse is administered a dose of 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶, 20×10⁶, or more engineered immune cells.

In some embodiments, the mouse is administered one dose of a therapeutic agent. In some embodiments, the mouse is administered 1-2, 1-3,1-4, 1-5, 2-3, 2-4, 2-5, 3-4, 3-5, or 4-5 doses (e.g., 2, 3, 4, 5, or more doses) a therapeutic agent. In some embodiments, the initial dose is divided into two or more smaller doses to mitigate the risk of side effects (e.g., instead of administering one initial dose, half the initial dose is administered twice). The time between administrations can be, for example, 12 hours, 1 day, 2 days, 3 days, 4 days, 5 days, 6 days, 7 days, 8 days, 9 days, 10 days, or more. In some embodiments, the time between administrations is 1-2 days, 1-3 days, 1-4 days, 1-5 days, 1-6, days, 1-7 days, 1-8 days, 1-9 days, 1-10 days, 2-3 days, 2-4 days, 2-5 days, 2-6 days, 2-7 days, 2-8 days, 2-9 days, 2-10 days, 3-4 days, 3-5 days, 3-6 days, 3-7 days, 3-8 days, 3-9 days, 3-10 days, 4-5 days, 4-6 days, 4-7 days, 4-8 days, 4-9 days, 4-10 days, 5-6 days, 5-7 days, 5-8 days, 5-9 days, 5-10 days, 6-7 days, 6-8 days, 6-9 days, 6-10 days, 7-8 days, 7-9 days, 7-10 days, 8-9 days, 8-10 days, or 9-10 days.

In some embodiments, the methods comprise assessing the efficacy and/or toxicity of any one of the a therapeutic agents described herein. In some embodiments, 2, 3, 4, 5, or 6 different types of therapeutic agents are assessed simultaneously.

In some embodiments, the human cells (e.g., PBMCs) from a T cell negative fraction are engrafted before the therapeutic agent is administered. For example, in some embodiments, the human cells (e.g., PBMCs) from a T cell negative fraction are engrafted immediately before the therapeutic agent is administered. In some embodiments, the human cells (e.g., PBMCs) from a T cell negative fraction are engrafted 0.5-10 days before the therapeutic agent is administered, e.g., 0.5 days, 1 day, 1.5 days, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, or 10 days before the therapeutic agent is administered. In some embodiments, the human cells (e.g., PBMCs) from a T cell negative fraction are administered 1-2 days, 1-3 days, 1-4 days, 1-5 days, 1-6 days, 1-7 days, 1-8, days, 1-9 days, 1-10 days, 2-3 days, 2-4 days, 2-5 days, 2-6 days, 2-7 days, 2-8 days, 2-9 days, 2-10 days, 3-4 days, 3-5 days, 3-6 days, 3-7 days, 3-8 days, 3-9 days, 3-10 days, 4-5 days, 4-6 days, 4-7 days, 4-8 days, 4-9 days, 4-10 days, 5-6 days, 5-7 days, 5-8 days, 5-9 days, 5-10 days, 6-7 days, 6-8 days, 6-9 days, 6-10 days, 7-8 days, 7-9 days, 7-10 days, 8-9 days, 8-10 days, or 9-10 days before the therapeutic agent is administered.

As will be appreciated by those of skill in the art, the cells and/or agents may be delivered to a mouse using any applicable route of administration. Exemplary routes of administration include, but not limited to, intravenous (e.g., via tail vein), subcutaneous, intrafemoral, intraventricular, intracardial, and intraperitoneal routes of administration. In some embodiments, the route of administration is intravenous injection via tail vein.

In some embodiments, the human cells (e.g., PBMCs) from a T cell negative fraction and the immune cells for engineered immune cell (e.g., T cell, B cell, or NK cell) therapy are from the same subject (the two cell types are autologous). In other embodiments, the human cells (e.g., PBMCs) from a T cell negative fraction and the immune cells are from different subjects (the two cell types are allogeneic). For example, the models described herein may be used to test a universal allogeneic engineered immune cell (e.g., T cell, B cell, or NK cell) therapy. In some embodiments, the human cells (e.g., PBMCs) from a T cell negative fraction, the immune cells (e.g., T cells, B cells, NK cells), and the tumor cells are from the same subject. In some embodiments, the human cells (e.g., PBMCs) from a T cell negative fraction, immune cells (e.g., T cells, B cells, NK cells), and tumor cells are from two or more subjects (e.g., the human cells (e.g., PBMCs) from a T cell negative fraction and immune cells are from one subject and the tumor cells are from a different subject; the human cells (e.g., PBMCs) from a T cell negative fraction and the tumor cells are from one subject and the immune cells are from a different subject; or the immune cells and the tumor cells are from one subject and the human cells (e.g., PBMCs) from a T cell negative fraction are from a different subject). In some embodiments, a subject from which the human cells (e.g., PBMCs) from a T cell negative fraction and/or immune cells (e.g., T cells, B cells, NK cells) are obtained is a human subject. Other mammals are contemplated herein.

Following administration of a therapeutic agent, a candidate agent effective for the treatment of CRS treatment (e.g., anti-cytokine treatment) may be administered (e.g., to prevent or reduce the effects of CRS). In some embodiments, the mouse models are used to determine whether a candidate CRS treatment will eliminate or reduce CRS in response to a therapeutic agent, as described herein. In some embodiments, the CRS treatment is administered simultaneously with the therapeutic agent. In some embodiments, the CRS treatment is administered 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, or more after the therapeutic agent has been administered. In some embodiments, the CRS treatment is administered 1-5 minutes, 1-10 minutes, 1-15 minutes, 1-20 minutes, 1-30 minutes, 1-45 minutes, 1-60 minutes, 2-5 minutes, 2-10 minutes, 2-15 minutes, 2-20 minutes, 2-30 minutes, 2-45 minutes, 2-60 minutes, 3-5 minutes, 3-10 minutes, 3-15 minutes, 3-20 minutes, 3-30 minutes, 3-45 minutes, 3-60 minutes, 4-5 minutes, 4-10 minutes, 4-15 minutes, 4-20 minutes, 4-30 minutes, 4-45 minutes, 4-60 minutes, 5-10 minutes, 5-20 minutes, 5-30 minutes, 5-45 minutes, 5-60 minutes, 10-20 minutes, 10-30 minutes, 10-40 minutes, 10-50 minutes, 10-60 minutes, 15-30 minutes, 15-45 minutes, 15-60 minutes, 30-45 minutes, 30-60 minutes, 1-2 hours, 1-3 hours, 1-4 hours, 1-5 hours, 1-6 hours, 1-7 hours, 1-8 hours, 1-9 hours, 1-10 hours, 1-11 hours, 1-12 hours, 2-3 hours, 2-4 hours, 2-5 hours, 2-6 hours, 2-7 hours, 2-8 hours, 2-9 hours, 2-10 hours, 2-11 hours, 2-12 hours, 3-4 hours, 3-5 hours, 3-6 hours, 3-7 hours, 3-8 hours, 3-9 hours, 3-10 hours, 3-11 hours, 3-12 hours, 4-5 hours, 4-6 hours, 4-7 hours, 4-8 hours, 4-9 hours, 4-10 hours, 4-11 hours, 4-12 hours, 5-6 hours, 5-7 hours, 5-8 hours, 5-9 hours, 5-10 hours, 5-11 hours, 5-12 hours, 6-7 hours, 6-8 hours, 6-9 hours, 6-10 hours, 6-11 hours, 6-12 hours, 7-8 hours, 7-9 hours, 7-10 hours, 7-11 hours, 7-12 hours, 8-9 hours, 8-10 hours, 8-11 hours, 8-12 hours, 9-10 hours, 9-11 hours, 9-12 hours, 10-11 hours, 10-12 hours, 11-12 hours, 12-16 hours, 12-18 hours, 12-20 hours, 12-24 hours, 1-2 days, 1-3 days, 1-4 days, 1-5 days, 1-6 days, 1-7 days, 1-8 days, 1-9 days, 1-10 days, 1-11 days, 1-12 days, 2-3 days, 2-4 days, 2-5 days, 2-6 days, 2-7 days, 2-8 days, 2-9 days, 2-10 days, 2-11 days, 2-12 days, 3-4 days, 3-5 days, 3-6 days, 3-7 days, 3-8 days, 3-9 days, 3-10 days, 3-11 days, 3-12 days, 4-5 days, 4-6 days, 4-7 days, 4-8 days, 4-9 days, 4-10 days, 4-11 days, 4-12 days, 5-6 days, 5-7 days, 5-8 days, 5-9 days, 5-10 days, 5-11 days, 5-12 days, 6-7 days, 6-8 days, 6-9 days, 6-10 days, 6-11 days, 6-12 days, 7-8 days, 7-9 days, 7-10 days, 7-11 days, 7-12 days, 8-9 days, 8-10 days, 8-11 days, 8-12 days, 9-10 days, 9-11 days, 9-12 days, 10-11 days, 10-12 days, or 11-12 days after the therapeutic agent has been administered. In some embodiments, the CRS treatment is administered prophylactically, such as 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 5 hours, 6 hours, 7 hours, 8 hours, 9 hours, 10 hours, 11 hours, 12 hours, 1 day, 2 days, 3 days, or 4 days before the therapeutic agent is administered. In some embodiments, the CRS treatment is administered 1-5 minutes, 1-10 minutes, 1-15 minutes, 1-20 minutes, 1-30 minutes, 1-45 minutes, 1-60 minutes, 2-5 minutes, 2-10 minutes, 2-15 minutes, 2-20 minutes, 2-30 minutes, 2-45 minutes, 2-60 minutes, 3-5 minutes, 3-10 minutes, 3-15 minutes, 3-20 minutes, 3-30 minutes, 3-45 minutes, 3-60 minutes, 4-5 minutes, 4-10 minutes, 4-15 minutes, 4-20 minutes, 4-30 minutes, 4-45 minutes, 4-60 minutes, 5-10 minutes, 5-20 minutes, 5-30 minutes, 5-45 minutes, 5-60 minutes, 10-20 minutes, 10-30 minutes, 10-40 minutes, 10-50 minutes, 10-60 minutes, 15-30 minutes, 15-45 minutes, 15-60 minutes, 30-45 minutes, 30-60 minutes, 1-2 hours, 1-3 hours, 1-4 hours, 1-5 hours, 1-6 hours, 1-7 hours, 1-8 hours, 1-9 hours, 1-10 hours, 1-11 hours, 1-12 hours, 2-3 hours, 2-4 hours, 2-5 hours, 2-6 hours, 2-7 hours, 2-8 hours, 2-9 hours, 2-10 hours, 2-11 hours, 2-12 hours, 3-4 hours, 3-5 hours, 3-6 hours, 3-7 hours, 3-8 hours, 3-9 hours, 3-10 hours, 3-11 hours, 3-12 hours, 4-5 hours, 4-6 hours, 4-7 hours, 4-8 hours, 4-9 hours, 4-10 hours, 4-11 hours, 4-12 hours, 5-6 hours, 5-7 hours, 5-8 hours, 5-9 hours, 5-10 hours, 5-11 hours, 5-12 hours, 6-7 hours, 6-8 hours, 6-9 hours, 6-10 hours, 6-11 hours, 6-12 hours, 7-8 hours, 7-9 hours, 7-10 hours, 7-11 hours, 7-12 hours, 8-9 hours, 8-10 hours, 8-11 hours, 8-12 hours, 9-10 hours, 9-11 hours, 9-12 hours, 10-11 hours, 10-12 hours, 11-12 hours, 12-16 hours, 12-18 hours, 12-20 hours, 12-24 hours, 1-2 days, 1-3 days, 1-4 days, 2-3 days, 2-4 days, or 3-4 days, before the therapeutic agent has been administered.

After the humanized mouse is administered the therapeutic agent (and optionally, a CRS treatment), the mouse may be observed to assess the efficacy of the therapeutic agent. As used herein, “efficacy” refers to the ability of the therapy administered to a subject to produce a therapeutic effect in the subject. In some embodiments, the therapy comprises a therapeutic agent. In some embodiments, the therapy comprises a therapeutic agent and anti-cytokine therapy. In some embodiments, the mouse models are used to determine whether a candidate CRS treatment will eliminate or reduce CRS in response to a specific therapeutic agent, as described herein. By “eliminate,” it is meant that the CRS treatment reduces the level of circulating cytokines following administration of the therapeutic agent in the mouse to the level of circulating cytokines present in the mouse prior to administration of the therapeutic agent or to the level of circulating cytokines in a control mouse that did not receive the therapeutic agent. By “reduce,” it is meant that the CRS treatment reduces the level of circulating cytokines following administration of the therapeutic agent in the mouse to a level that is lower than would be found in a mouse administered the therapeutic agent without the CRS treatment. In some embodiments, the CRS treatment reduces the circulating cytokine level (e.g., the cytokine level of one or more cytokines) 5%, 10%, 15%, 20%, 25%, 30%, 35%, 40%, 45%, 50%, 55%, 60%, 65%, 70%, 75%, 80%, 85%, 90%, 95%, or more. In some embodiments, the circulating cytokine level (e.g., one or more cytokines) in the mouse is reduced 10-20%, 10-30%, 10-40%, 10-50%, 10-60%, 10-70%, 10-80%, 10-90%, 10-100%, 20-30%, 20-40%, 20-50%, 20-60%, 20-70%, 20-80%, 20-90%, 20-100%, 30-40%, 30-50%, 30-60%, 30-70%, 30-80%, 30-90%, 30-100%, 40-50%, 40-60%, 40-70%, 40-80%, 40-90%, 40-100%, 50-60%, 50-70%, 50-80%, 50-90%, 50-100%, 60-70%, 60-80%, 60-90%, 60-100%, 70-80%, 70-90%, 70-100%, 80-90%, 80-100%, or 90-100%.

To determine the efficacy of the therapeutic agent, tumor growth can be monitored, for example, with in vivo bioluminescence imaging (BLI) as described herein, genomic studies, histology studies, or with any other method of measuring or approximating the volume of a tumor. Efficacy may be determined by using the Response Evaluation Criteria in Solid Tumors (RECIST) criteria, the 3-category method, the 4-response mRECIST criterion, and the 5-category method (Eisenhauer et al., Eur J Cancer, 2009, 45 (2): 228-247; Bertotti et al., Nature. 2015;526 (7572): 263-7; Gao et al., Nat Med. 2015;21 (11): 1318-25; Houghton et al., Pediatr Blood Cancer. 2007; 49 (7): 928-40). Other measurements of efficacy relate to tumor volume and include, but are not limited to, progression-free survival, tumor volume doubling time, relative tumor volume (RTV), tumor growth inhibition (changes in tumor volume relative to initial tumor volume), and tumor growth rate. Progression-free survival is the length of time during and following treatment when the subject has the disease, but it does not get worse (e.g., the amount of time, during and after engineered immune cell (e.g., T cell, B cell, or NK cell) therapy, that the tumor does not grow). Tumor volume doubling time

(DT) is the amount of time it takes the tumor volume to double (faster doubling times indicate a more malignant tumor) and typically determined from two volume estimations with measurement time intervals comparable with or shorter than DT. Relative tumor volume is the relative in tumor volume over time and is calculated as: (absolute tumor volume on day X)×(100/absolute tumor volume on day 0). Day 0 is the day the engineered immune cell (e.g., T cell, B cell, or NK cell) therapy begins. Similarly, tumor growth inhibition (TGI), which is expressed as a percentage, examines the changes in tumor volume relative to the initial tumor volume using the formula: (1-(mean volume of treated tumors)/(mean volume of control tumors))×100%. Tumor growth rate is estimated using a variety of different models. For an exponentially growing tumor, the growth rate is proportional to its volume: (1/V)×(dV/dt), where V is the volume of the tumor, and dV and dt are the change in volume and time, respectively.

The tumor volume can be measured any number of times throughout the time course experiment (e.g., 1, 2, 3, 4, 5, 6, 7, 8, 9, 10 or more times). In some embodiments, the tumor volume is measured 1-2 times, 1-3 times, 1-4 time, 1-5 times, 1-6 times, 1-7 times, 1-8 times, 1-9 times, 1-10 times, 2-4 times, 2-6 times, 2-8 times, 2-10 times, 3-6 times, 3-8 times, 3-10 times, 4-6 times, 4-8 times, 4-10 times, 5-8 times, 5-10 times, 6-8 times, 6-10 times, 7-10 times, 8-10 times, or 9-10 times. The tumor volume may be measured over time, for example 1, 2, 3, 4, 5, 6, 7, 8, 9, 10, 11, 12, 13, 14, 15, 16, 17, 18, 19, 20, 21 or more days after engraftment with the tumor cells. In some embodiments, the tumor volume is measured 1-2 days, 1-3 days, 1-4 days, 1-5 days, 1-6 days, 1-7 days, 1-8 days, 1-9 days, 1-10 days, 1-11 days, 1-12 days, 2-3 days, 2-4 days, 2-5 days, 2-6 days, 2-7 days, 2-8 days, 2-9 days, 2-10 days, 2-11 days, 2-12 days, 3-4 days, 3-5 days, 3-6 days, 3-7 days, 3-8 days, 3-9 days, 3-10 days, 3-11 days, 3-12 days, 4-5 days, 4-6 days, 4-7 days, 4-8 days, 4-9 days, 4-10 days, 4-11 days, 4-12 days, 5-6 days, 5-7 days, 5-8 days, 5-9 days, 5-10 days, 5-11 days, 5-12 days, 6-7 days, 6-8 days, 6-9 days, 6-10 days, 6-11 days, 6-12 days, 7-8 days, 7-9 days, 7-10 days, 7-11 days, 7-12 days, 8-9 days, 8-10 days, 8-11 days, 8-12 days, 9-10 days, 9-11 days, 9-12 days, 10-11 days, 10-12 days, 10-14 days, 10-16 days, 10-18 days, 10-20 days, 10-21 days, 12-14 days, 12-16 days, 12-18 days, 12-20 days, 12-21 days, 14-16 days, 14-18 days, 14-20 days, 14-21 days, 15-16 days, 15-18 days, 15-20 days, 15-21 days, 16-18 days, 16-20 days, 16-21 days, 17-19 days, 17-21 days, 18-19 days, 18-20 days, 18-21 days, 19-20 days, 19-21 days, or 20-21 days.

In some embodiments, the change in tumor volume is indicative of the efficacy of the therapeutic agent (and, optionally, the CRS treatment). In some embodiments, the tumor volume in a mouse treated with the therapeutic agent may be compared to the tumor volume in a mouse that was not treated with the therapeutic agent. In some embodiments, the tumor volume is reduced 5%, 10%, 15%, 20%, 25%, 30%, 35%, 40%, 45%, 50%, 55%, 60%, 65%, 70%, 75%, 80%, 85%, 90%, 95%, or more relative to a mouse that did not receive the therapeutic agent or relative to an earlier time point. In some embodiments, the tumor volume is reduced 10-20%, 10-30%, 10-40%, 10-50%, 10-60%, 10-70%, 10-80%, 10-90%, 10-100%, 20-30%, 20-40%, 20-50%, 20-60%, 20-70%, 20-80%, 20-90%, 20-100%, 30-40%, 30-50%, 30-60%, 30-70%, 30-80%, 30-90%, 30-100%, 40-50%, 40-60%, 40-70%, 40-80%, 40-90%, 40-100%, 50-60%, 50-70%, 50-80%, 50-90%, 50-100%, 60-70%, 60-80%, 60-90%, 60-100%, 70-80%, 70-90%, 70-100%, 80-90%, 80-100%, or 90-100% relative to a mouse that did not receive the therapeutic agent or relative to an earlier time point.

CRS induction may also be monitored through body weight measurement, as acute toxicity relates to significant mouse body weight loss. Further, clinical observations may be indicative of CRS. Examples of clinical observations relevant to CRS include: a hunched posture with tiptoe/abnormal gait, reduced activity (e.g., not moving unless being stimulated), and/or non-responsiveness to touch. Survival rate (and duration) may also be used to evaluate the efficacy of a human engineered immune cell (e.g., T cell, B cell, or NK cell) therapy (and/or anti-CRS treatment).

Efficacy may also be evaluated by examining cytokine-induced liver and kidney damage. This may be determined, for example, by a serum biochemical analysis of liver-kidney function, such as measuring levels of aspartate transaminase (AST), albumin, total bilirubin, creatinine and blood urea nitrogen.

In some embodiments, the change in liver weight of preclinical mouse model is indicative of the efficacy of the therapeutic agent (and, optionally, the CRS treatment). A healthy mouse's liver weight is approximately 5% of its body weight. Injection of tumor cells (e.g., Raji_Luc cells) leads to dissemination of the tumor cells to the liver, increasing the liver weight. By measuring the weight of the liver after administration of the therapeutic agent, one may determine whether the treatment effectively eliminated or reduced tumor cell accumulation in the liver.

Methods for Assessing Side Effects of Therapeutic Agents In some embodiments, the methods described herein may be used to assess the possible side effects of a therapeutic agent. Possible side effects include but are not limited to cytokine release syndrome (CRS), macrophage activation syndrome (MAS), neurotoxicity (encephalopathy syndrome), tumor lysis syndrome (TLS), anaphylaxis, on-target, off-tumor toxicity, and B cell aplasia.

With respect to CRS, certain cytokines can be measured in a blood sample from the humanized mouse model following administration of the therapeutic agent and/or CRS treatment. For example, the cytokine may be selected from the group consisting of IFN-γ, IL-10, 10, IL-6, IL-2, IL-4, and TNFα. The level of cytokine measured is indicative of the severity of immunotoxicity of the therapeutic agent. In some embodiments, the method further comprises determining that the severity of immunotoxicity of the therapeutic agent is high (e.g., the likelihood of CRS induction is high) when: an IFN-γ level in the mouse is ≥1,800 pg/ml±10%; an IL-10 level in the mouse is ≥120 pg/ml±10%; an IL-6 level in the mouse is ≥25 pg/ml±10%; an IL-2 level in the mouse is >80 pg/ml±10%; an IL-4 level in the mouse is ≥120 pg/ml±10%; TNFα level in the mouse is ≥120 pg/ml±10%; MCP-1 level in the mouse is ≥120 pg/ml±10%; GM-CSF level in the mouse is ≥600 pg/ml±10%; and IL8 level in the mouse is ≥15 pg/ml±10%.

Macrophage activation syndrome (MAS) or hemophagocytic lymphohistiocytosis (HLH), which clinically manifests as liver dysfunction, increased ferritin levels and, in some cases, decreased fibrinogen levels, may also be examined using the mouse models described herein. Macrophages mediate the major production of cytokines including IL-6, IL-1, and IFN-γ, and their activation (MAS) is thought to play a role in CRS (Hao et al., Experimental Hematology & Oncology, 2020, 9:15). Therefore, elevated levels of the three cytokines in the mouse model may indicate that that the engineered immune cell (e.g., T cell, B cell, or NK cell) therapy may result in MAS. Likewise, the mouse may be administered a MAS treatment to determine whether the treatment would prevent or reduce MAS in the human subject. Examples of MAS treatments include but are not limited to, glucocorticoids (e.g., methylprednisone, dexamethasone), cyclosporin A, etoposide, immunoglobulins, and cyclophosphamide.

Neurotoxicity (encephalopathy syndrome), or immune effector cell-associated neurotoxicity syndrome (ICANS) can develop approximately 5-17 days after engineered immune cell (e.g., T cell, B cell, or NK cell) therapy in humans, for example (Herlopian et al., Neurology, 2018, 91 (5): 227-229). It is characterized by global encephalopathy, aphasia, seizure/seizure-like activity, obtundation, tremor/myoclonus, and hallucinations. Subjects with neurotoxicity also have high levels of IFN-γ, IL-6, and TNF-α. Elevated levels of the three cytokines in the mouse model may indicate that the engineered immune cell (e.g., T cell, B cell, or NK cell) therapy may result in neurotoxicity. Therefore, in some embodiments, the mouse may be administered a neurotoxicity treatment to determine whether the treatment would prevent or reduce neurotoxicity in the human subject. Examples of neurotoxicity treatments include but are not limited to corticosteroids (e.g., dexamethasone, prednisone), anti-IL-6 antibodies (e.g., siltuximab), and platelet hypertransfusion.

### Assays

In some embodiments, the methods further comprise assaying one or more effect(s) of the therapeutic modality on the human cells and/or the mouse.

In some embodiments, the assaying comprises assaying for cell death (e.g., necrosis and/or apoptosis), inflammation, oxidative stress, alterations in cell morphology, alterations in cell function, accumulation of toxic substances, and changes in enzyme activity.

In some embodiments, the methods comprise assaying for cell death, which can lead to tissue damage and dysfunction. Cell death assays are used to measure and quantify different forms of cell death, such as apoptosis, necrosis, and autophagy. These assays help one understand the mechanisms and extent of cell death in various biological processes. Several commonly used cell death assays include the Annexin V/Propidium Iodide (PI) Assay, TUNEL Assay, Caspase Activity Assay, LDH Release Assay, MTT Assay, PI Exclusion Assay, and Caspase-Glo® Assays. The Annexin V/PI Assay distinguishes between early apoptotic and late-stage apoptotic or necrotic cells. Annexin V, labeled with a fluorescent marker, binds to phosphatidylserine, a marker for early apoptosis. Propidium iodide (PI) stains cells with compromised membranes, indicating late-stage apoptosis or necrosis. Flow cytometry is typically used to analyze the distribution of stained cells. The TUNEL (Terminal deoxynucleotidyl transferase dUTP nick end labeling) Assay detects DNA fragmentation, a characteristic of apoptosis. It involves labeling DNA strand breaks using a modified nucleotide that can be visualized using fluorescence microscopy or flow cytometry. This assay allows for the quantification of apoptotic cells within a population. Caspase Activity Assays measure the activity of specific caspases, enzymes involved in apoptosis. Using fluorescent or colorimetric substrates, these assays detect the cleavage of substrates by active caspases, generating a measurable signal. Caspase-3,-8, or-9 activity can be measured, indicating the activation of apoptotic pathways. The LDH (Lactate Dehydrogenase) Release Assay measures the release of LDH, an enzyme, into the culture medium upon cell membrane damage or disruption, which is characteristic of necrotic cell death. This assay quantifies the amount of LDH in the culture supernatant using a colorimetric or fluorometric assay, indicating compromised membrane integrity and cell death. The MTT (3-(4,5-Dimethylthiazol-2-yl)-2,5-diphenyltetrazolium Bromide) Assay measures cell viability based on the ability of living cells to reduce MTT, a yellow tetrazolium salt, to a purple formazan product. The formazan can be quantified spectrophotometrically, and a decrease in formazan production indicates reduced cell viability. The PI Exclusion Assay uses propidium iodide (PI), a DNA-intercalating fluorescent dye, to distinguish viable cells from non-viable ones. PI cannot penetrate intact cell membranes, so it only stains cells with compromised membrane integrity, such as necrotic cells. Flow cytometry or fluorescence microscopy can be used to analyze the stained cells. Caspase-Glo® Assays are luminescent assays that utilize a luminogenic caspase substrate. Upon caspase cleavage, the substrate emits a light signal. These assays can be specific to different caspases, such as caspase-3/7 or caspase-8, providing a sensitive and quantitative measurement of caspase activity, indicating apoptotic cell death.

In some embodiments, the methods comprise assaying for inflammation, which can lead to swelling, redness, and pain. Inflammation assays are widely used to study and measure the presence and extent of inflammation, a complex immune response that occurs in various tissues and organs. These assays help one understand the underlying mechanisms of inflammation, identify potential therapeutic targets, and evaluate the efficacy of anti-inflammatory treatments. Several commonly used inflammation assays include cytokine analysis, cell migration assays, leukocyte adhesion assays, nitric oxide assays, myeloperoxidase assays, histological staining, reactive oxygen species assays, and inflammatory gene expression analysis. Cytokine analysis is a key approach to quantify inflammation. This assay involves measuring the levels of specific cytokines, such as interleukins (IL), tumor necrosis factor-alpha (TNF-α), and interferons (IFN), in biological samples using techniques like ELISA, multiplex immunoassays, or protein arrays. By assessing cytokine profiles, one can gain insights into the inflammatory processes occurring in different tissues. Cell migration assays are utilized to study the migratory capacity of immune cells, such as neutrophils or monocytes, in response to inflammatory stimuli. Transwell assays or scratch assays provide valuable information about immune cell migration and infiltration into inflamed tissues. Leukocyte adhesion assays focus on measuring the adhesion of leukocytes (white blood cells) to endothelial cells, which is a critical step in the inflammatory response. By employing flow chamber assays or static adhesion assays, one can evaluate the adhesion properties of leukocytes under inflammatory conditions, contributing to our understanding of leukocyte-endothelial interactions. Nitric oxide (NO) assays are employed to measure the production of nitric oxide, a signaling molecule involved in inflammation. Griess reagent-based assays or fluorescent probes allow one to assess the levels of nitric oxide, which serves as an indicator of inflammatory activity. Myeloperoxidase (MPO) assays are used to quantify the presence of neutrophils or the extent of inflammation in tissues. MPO is an enzyme released by activated neutrophils and macrophages during inflammation, and measuring MPO activity provides insights into the level of neutrophil infiltration and inflammatory activity. Histological staining techniques, such as hematoxylin and eosin (H&E) staining, play a crucial role in visualizing and assessing inflammatory changes in tissue samples. By examining cellular and tissue alterations, including immune cell infiltration, tissue damage, and edema, one can identify and characterize inflammatory responses. Reactive oxygen species (ROS) assays detect the presence of reactive oxygen species generated during inflammation. Fluorescent probes, such as dichlorofluorescein diacetate (DCFDA), enable the measurement of ROS production in cells or tissues, indicating the presence and extent of inflammation. Inflammatory gene expression analysis involves quantifying the expression levels of specific inflammatory genes, including cytokines, chemokines, and adhesion molecules. Techniques such as quantitative real-time polymerase chain reaction (qPCR) or gene expression microarrays allow one to assess gene expression patterns, providing insights into the molecular aspects of the inflammatory response.

In some embodiments, the methods comprise assaying for oxidative stress, which can damage cellular components and cause tissue dysfunction. Oxidative stress assays are valuable tools used to measure and assess the levels of reactive oxygen species (ROS) and oxidative damage within cells and tissues. These assays provide insights into the oxidative stress status, which is implicated in various physiological and pathological conditions. Several commonly used oxidative stress assays include the DCFDA assay, NBT assay, total antioxidant capacity assay, lipid peroxidation assay, protein carbonyl assay, glutathione assay, DNA oxidation assay, and mitochondrial membrane potential assay. The DCFDA assay is a widely used fluorometric assay that measures intracellular ROS levels. DCFDA, a non-fluorescent probe, is oxidized by ROS to form the fluorescent compound dichlorofluorescein (DCF). The fluorescence intensity of DCF is proportional to the level of ROS within the cells and can be quantified using fluorescence microscopy or flow cytometry.

The NBT assay detects superoxide anions, a type of ROS, by their ability to reduce NBT to formazan crystals. The intensity of the resulting blue formazan precipitate is proportional to the level of superoxide anions generated. This assay is commonly used in histochemical analysis to visualize and quantify superoxide production in tissues. Total antioxidant capacity assays measure the overall antioxidant capacity of biological samples, encompassing both enzymatic and non-enzymatic antioxidants. These assays evaluate the sample's ability to scavenge free radicals or prevent oxidative damage. Methods such as the Trolox equivalent antioxidant capacity (TEAC) assay and the ferric reducing antioxidant power (FRAP) assay are employed to determine the total antioxidant capacity. Lipid peroxidation assays assess the levels of lipid peroxidation products, such as malondialdehyde (MDA), as an indicator of oxidative damage to lipids. The thiobarbituric acid reactive substances (TBARS) assay or MDA assay is commonly used to measure lipid peroxidation, a common consequence of oxidative stress. Protein carbonyl assays detect the presence of carbonylated proteins, which result from protein oxidation due to oxidative stress. These assays derivatize the carbonyl groups with 2,4-dinitrophenylhydrazine (DNPH) and quantify the protein-bound DNPH, providing a measurement of protein oxidation using spectrophotometry. Glutathione assays evaluate the levels of reduced (GSH) and oxidized (GSSG) forms of glutathione, an important intracellular antioxidant. These assays, such as the enzymatic recycling method or Ellman's reagent-based assay, provide insights into the cellular antioxidant capacity and redox balance. DNA oxidation assays detect and quantify DNA damage resulting from oxidative stress. The comet assay (single-cell gel electrophoresis) and 8-hydroxy-2′-deoxyguanosine (8-OHdG) assay are commonly used to assess DNA damage, including oxidized bases and DNA strand breaks caused by oxidative stress. Mitochondrial membrane potential assays measure changes in mitochondrial function resulting from oxidative stress. Fluorescent dyes such as JC-1 or TMRE (tetramethylrhodamine ethyl ester) are employed to evaluate alterations in mitochondrial membrane potential using fluorescence microscopy or flow cytometry.

In some embodiments, the methods comprise assaying for alterations in cell morphology, for example, changes in the size, shape, and structure of cells, which can lead to tissue dysfunction. Assaying for alterations in cell morphology is an approach to study cellular changes associated with various biological processes or pathological conditions. By examining the structural characteristics and shape of cells, one can gain insights into cellular function, differentiation, disease progression, and response to treatments. Several commonly used methods enable the assessment of alterations in cell morphology. Light microscopy is a fundamental technique for visualizing and assessing cell morphology. Brightfield microscopy provides high-resolution images that allow one to examine overall cell shape, size, and features such as organelles and cytoplasmic structures. Phase contrast microscopy and differential interference contrast (DIC) microscopy enhance contrast and improve the visualization of cellular details, especially for transparent or unstained cells. Fluorescence microscopy utilizes fluorescent dyes or genetically encoded fluorescent proteins to label specific cellular components or structures. By targeting specific molecules, one can visualize and study alterations in cell morphology, such as changes in cytoskeletal organization, organelle distribution, or nuclear morphology. Techniques like immunofluorescence staining and live cell imaging provide valuable information about cellular dynamics and structural changes. Electron microscopy (EM) offers high-resolution imaging of cellular structures at the ultrastructural level. Transmission electron microscopy (TEM) provides detailed views of cellular organelles, membranes, and cytoplasmic components. Scanning electron microscopy (SEM) enables three-dimensional visualization of cell surfaces and can reveal alterations in cell shape, surface morphology, or the presence of cellular protrusions. Cytospin and cell smear techniques involve spreading cells onto glass slides, followed by fixation and staining. These methods allow one to examine cell morphology under a microscope and assess features such as cell size, shape, nuclear characteristics, and the presence of cellular inclusions or abnormalities. Staining techniques like Giemsa, Wright-Giemsa, or Papanicolaou stains can be employed to enhance cellular details and facilitate the identification of specific cell types. High-content imaging combines automated microscopy with image analysis software to quantitatively assess alterations in cell morphology and subcellular structures. This approach enables large-scale screening of cellular phenotypes, measuring parameters such as cell shape, size, texture, or fluorescent intensity. High-content imaging is particularly useful for studying cellular responses to treatments, genetic perturbations, or disease-related processes. Advanced image analysis software tools are available to quantify alterations in cell morphology from microscopy images. These tools allow one to measure parameters such as cell area, perimeter, circularity, aspect ratio, and intensity distribution. By comparing these morphological parameters between different experimental conditions or cell populations, one can identify and quantify changes in cell shape or structure.

In some embodiments, the methods comprise assaying for alterations in cell function, which can lead to tissue dysfunction and organ failure. Assaying for alterations in cell function is crucial for understanding cellular processes, evaluating the effects of treatments or genetic modifications, and investigating disease mechanisms. Various techniques and assays are available to assess changes in cellular function. These methods provide valuable insights into cellular behavior, signaling pathways, metabolism, and overall cellular health. Enzyme activity assays measure the activity levels of specific enzymes involved in various cellular processes. By employing specific substrates that undergo measurable changes upon enzymatic reactions, one can assess alterations in metabolic pathways, signal transduction, or other enzymatic processes. Calcium imaging techniques enable the monitoring of intracellular calcium levels, which play a critical role in cellular signaling and the regulation of various cellular functions. Fluorescence microscopy using calcium-sensitive dyes allows one to assess alterations in calcium dynamics, providing insights into processes such as neuronal signaling, muscle contraction, or cell communication. Electrophysiological techniques, such as patch-clamp recordings, measure the electrical activity of cells. These techniques assess alterations in membrane potential, ion channel activity, action potentials, synaptic transmission, or other electrical properties of cells. Electrophysiology is widely used in neuroscience and cardiac research to study cellular excitability and function. Metabolic assays measure various aspects of cellular metabolism, such as glucose uptake, ATP production, or oxygen consumption. By utilizing specific substrates or indicators, these assays allow one to quantify alterations in cellular energy metabolism or metabolic pathways. Cell proliferation and viability assays evaluate changes in cell growth, division, or survival. Techniques like MTT assays, cell counting, or live/dead staining provide quantitative or qualitative measurements of alterations in cell proliferation or viability in response to treatments, genetic modifications, or environmental conditions. Analyzing cell signaling pathways reveals changes in cellular responses or signaling cascades. Techniques such as Western blotting, immunofluorescence staining, or ELISA can be used to analyze protein expression, phosphorylation levels, or activation states of specific signaling molecules. These methods elucidate alterations in signaling pathways involved in processes like cell growth, differentiation, or immune responses. Functional imaging techniques, such as fMRI or PET, are used to study alterations in cell function in living organisms or tissues. These non-invasive imaging methods provide insights into functional changes in organs, tissues, or specific cell types and are commonly used in neuroscience, cardiovascular research, or oncology. Flow cytometry allows for the simultaneous analysis of multiple cellular parameters. By using fluorescently labeled antibodies or dyes, flow cytometry assesses alterations in cell surface markers, intracellular protein expression, cell cycle distribution, or apoptosis. It provides quantitative information on alterations in various cellular functions within complex cell populations.

In some embodiments, the methods comprise assaying for accumulation of toxic substances, which can lead to tissue damage and dysfunction. Assaying for the accumulation of toxic substances is essential for studying the impact of various chemicals, pollutants, or drugs on cells and organisms. These assays provide valuable insights into toxicological mechanisms, the potential adverse effects of substances, and the efficacy of detoxification or protective interventions. Several commonly used methods enable the assessment of toxic substance accumulation. Analytical techniques such as HPLC (High-Performance Liquid Chromatography) and GC-MS (Gas Chromatography-Mass Spectrometry) allow for the identification and quantification of toxic substances. HPLC separates and quantifies a wide range of compounds, providing information about their accumulation levels. GC-MS combines gas chromatography and mass spectrometry to detect and characterize toxic substances based on their mass-to-charge ratio, particularly for volatile or semi-volatile compounds. Fluorescence spectroscopy measures the emission of fluorescent light from a sample upon excitation with specific wavelengths. By using fluorescent probes or dyes, fluorescence spectroscopy can assess the accumulation of toxic substances by monitoring changes in fluorescence intensity or emission spectra. These probes selectively bind to or react with specific toxic compounds, offering a direct readout of their accumulation. Enzyme activity assays evaluate alterations in enzyme function caused by toxic substances. These assays employ specific substrates and indicators to measure enzyme activity, providing insights into the impact of toxic substances on cellular processes. Some toxic compounds can interfere with cellular enzymes, inhibiting their activity or leading to abnormal enzymatic reactions. Immunohistochemistry and immunofluorescence techniques use specific antibodies to detect and visualize the accumulation of toxic substances in tissues or cells. By targeting specific antigens or epitopes related to toxic compounds, these techniques allow for the spatial identification and localization of accumulated toxic substances. Cell-based assays utilize specific fluorescent dyes or probes to assess the accumulation of toxic substances in cultured cells. These assays employ fluorescence microscopy or flow cytometry to quantify the accumulation of toxic compounds, offering insights into their cellular uptake, distribution, and metabolism. Tissue analysis can be employed to study the accumulation of toxic substances in vivo. Tissue analysis involves the extraction and quantification of toxic compounds from organs or biological fluids, enabling one to evaluate their accumulation levels and distribution patterns in different tissues or body compartments. Indirect assays target specific physiological or biochemical changes caused by toxic substances. Assays measuring oxidative stress markers, DNA damage, or metabolic alterations can indirectly infer the presence and accumulation of toxic compounds. These changes serve as indicators of the effects of toxic substances on cells or organisms.

In some embodiments, the methods comprise assaying for changes in enzyme activity, which can lead to tissue dysfunction and organ failure. Assaying for changes in enzyme activity can be used for studying enzymatic processes, assessing the impact of various factors on enzyme function, and identifying potential disease-related alterations. Several commonly used methods allow one to quantitatively measure the catalytic activity of enzymes and detect changes in their function. Spectrophotometric assays utilize the measurement of absorbance or color change to quantify enzyme activity. These assays often involve enzymatic reactions that produce or consume specific substrates, resulting in changes in light absorption. By monitoring the absorbance or color intensity, one can determine the rate of enzymatic activity. Examples include the use of substrates such as NADH or NADPH, which exhibit changes in absorbance upon enzymatic reactions. Fluorometric assays rely on the detection of fluorescence emitted by a substrate or product of an enzymatic reaction. Fluorescent molecules can be designed to interact specifically with certain enzymes, generating fluorescence signals upon enzymatic activity. By measuring the fluorescence intensity, one can quantify enzyme activity. Fluorometric assays are highly sensitive and often used in high-throughput screening. Radiometric assays involve the use of radioactive isotopes to track enzymatic reactions. Radioactive substrates or cofactors are used in the enzymatic reaction, and the radioactivity of the reaction products is measured using techniques such as liquid scintillation counting. These assays provide high sensitivity but require special precautions due to the use of radioactive materials. Enzyme-Linked Immunosorbent Assay (ELISA) utilizes the specificity of antibodies to detect and quantify enzyme activity. In these assays, enzymes are conjugated to antibodies or antigens, and their activity is measured through the detection of an enzymatic reaction product. ELISA is widely used for the quantification of various enzymes or enzyme activities in biological samples. Gel electrophoresis techniques, such as zymography or native gel electrophoresis, are used to assess changes in enzyme activity based on their mobility in a gel matrix. Enzymes are separated based on their size, charge, or activity, and subsequent staining or activity-based detection methods reveal alterations in enzyme activity. Kinetic assays measure the rate of enzyme-catalyzed reactions under varying substrate concentrations or reaction conditions. These assays determine key kinetic parameters such as the Michaelis-Menten constant (Km) and maximum reaction velocity (Vmax), providing insights into enzyme-substrate interactions and the impact of factors on enzyme activity. Common kinetic assays include the Lineweaver-Burk plot and steady-state kinetic analysis. Mass spectrometry can be utilized to quantify enzyme activity by measuring the consumption or production of metabolites involved in enzymatic reactions. Isotope-labeled substrates or reactants can be introduced, and the change in isotopic ratio is detected using mass spectrometry. This approach allows for precise measurements of enzyme activity and can be applied to complex enzymatic pathways. Activity-based probes are small molecules that selectively react with active enzyme sites. These probes covalently modify active enzymes, allowing for their subsequent detection or isolation. Activity-based probes provide a powerful method for profiling enzyme activity in complex biological systems.

In some embodiments, the assaying comprises assaying for reduced tumor volume, tumor necrosis, changes in tumor density, decreased tumor markers, and slowed tumor growth.

In some embodiments, the methods comprise assaying for reduced tumor volume. One of the primary goals of cancer treatment is to shrink the tumor size, and a reduction in tumor volume is often considered a sign of treatment success. Assaying for reduced tumor volume is a critical aspect of evaluating the effectiveness of anti-cancer therapies and monitoring the progression of cancer treatment. Several methods are used to assess changes in tumor size and volume, providing valuable information about treatment response and disease progression. Imaging techniques, including computed tomography (CT), magnetic resonance imaging (MRI), or ultrasound, are commonly employed to visualize and measure tumor size and volume. These non-invasive imaging modalities generate detailed images of the tumor and surrounding tissues, enabling accurate measurements of tumor dimensions and volume changes over time. Tumor biopsy and histopathological assessment play a crucial role in evaluating tumor size reduction. Tumor biopsy involves the removal of a tissue sample from the tumor for microscopic examination. Histopathological analysis allows for the evaluation of tumor size, growth patterns, and cellular characteristics. Reduction in tumor size and changes in cellular features observed through histopathology can indicate response to treatment. Caliper measurements are frequently used in preclinical models, where tumors are directly accessible and measurable. This method involves manually measuring tumor size using calipers or rulers. By measuring the longest diameter (length) and the perpendicular diameter (width) of the tumor, the volume can be estimated using mathematical formulas. Tumor weight serves as an indirect measure of tumor volume reduction. Tumors can be surgically excised and weighed before and after treatment, allowing for the determination of changes in tumor mass. Reduction in tumor weight indicates a decrease in tumor volume. Positron Emission Tomography (PET) imaging utilizes radiolabeled tracers that are selectively taken up by metabolically active tissues, including tumors. PET scans provide functional information on tumor metabolism and can be used to assess changes in tumor size and metabolic activity following treatment. The Response Evaluation Criteria in Solid Tumors (RECIST) is a standardized method for assessing tumor response in clinical trials. RECIST defines specific criteria based on changes in tumor size to categorize treatment response as complete response, partial response, stable disease, or progressive disease. It considers the longest diameter of target lesions to evaluate changes in tumor size. Advanced imaging software allows for 3D reconstruction and volumetric analysis of tumors. By segmenting the tumor region in imaging data, volumetric analysis provides a more precise and comprehensive assessment of tumor volume changes over time. Tumor markers are specific proteins or substances produced by tumor cells that can be measured in blood samples. Monitoring changes in tumor marker levels, such as PSA for prostate cancer or CEA for colorectal cancer, can provide indirect information about tumor response to treatment.

In some embodiments, the methods comprise assaying for tumor necrosis, which occurs when cancer cells die due to treatment, and can be seen as dark, dead tissue within the tumor on imaging studies. Assaying for tumor necrosis is an important aspect of evaluating the response to cancer treatments and understanding the pathological characteristics of tumors. Several methods are used to assess tumor necrosis and its extent, providing valuable information about treatment response and the nature of the tumor microenvironment. Histopathological examination of tumor tissue sections is a primary method for assessing tumor necrosis. Hematoxylin and eosin (H&E) staining allows pathologists to visualize tissue morphology and identify areas of necrosis within the tumor. Necrotic areas appear as pink amorphous material within the tumor sections. Analyzing the extent and patterns of necrosis provides important insights into the response to treatment. Immunohistochemistry (IHC) utilizes specific antibodies to detect and characterize proteins or markers associated with necrotic tissue. Antibodies against markers such as cleaved caspase-3, involved in the apoptosis pathway, can be used to identify apoptotic cells within necrotic areas. Other markers, such as hypoxia-inducible factor-1 alpha (HIF-1α), may indicate areas of tumor hypoxia, which can contribute to necrosis. Imaging techniques, including CT, MRI, or PET, provide non-invasive visualization of necrotic regions within tumors. Imaging features such as decreased enhancement, areas of low signal intensity, or reduced metabolic activity can indicate tumor necrosis. These techniques allow for the assessment of necrotic changes over time and can be useful for monitoring treatment response. Tumor markers, such as lactate dehydrogenase (LDH), can provide indirect information about tumor necrosis. Elevated levels of LDH in blood samples can indicate extensive tumor cell death, including necrosis. Monitoring changes in tumor marker levels can provide insights into the extent of tumor necrosis and treatment response. The analysis of circulating tumor DNA (ctDNA) in blood samples can also provide information about tumor necrosis. As tumor cells die, fragments of their DNA are released into the bloodstream. Detecting and quantifying ctDNA can be used to assess the presence of necrotic tumor cells and monitor their response to treatment. Molecular profiling techniques, such as gene expression analysis or genomic sequencing, can be employed to identify specific molecular signatures associated with tumor necrosis. These techniques provide insights into the molecular pathways and cellular processes underlying necrotic changes in tumors. Molecular profiling contributes to a better understanding of the mechanisms involved in tumor necrosis and can guide treatment strategies.

In some embodiments, the methods comprise assaying for changes in tumor density. Imaging studies such as CT or MRI can show changes in the density of the tumor after treatment, indicating the tumor is responding to treatment. Assaying for changes in tumor density is important for evaluating tumor characteristics, monitoring treatment response, and assessing disease progression. Tumor density refers to the composition and structural properties of the tumor mass, including the distribution of cells, extracellular matrix, blood vessels, and other components. Several methods are used to assess changes in tumor density, providing valuable insights into tumor biology. Computed Tomography (CT) imaging is commonly used to assess tumor density. It provides cross-sectional images of the tumor, allowing for the visualization and quantification of tissue density based on X-ray attenuation. CT scans can differentiate between different tissue densities, such as solid tumor regions, areas of necrosis, or cystic components. By analyzing the density distribution within the tumor, changes in tumor density can be identified. Magnetic Resonance Imaging (MRI) techniques can also provide information about tumor density. MRI utilizes magnetic fields and radio waves to generate detailed images of tissues. Different MRI sequences, such as T1-weighted or T2-weighted images, can provide insights into tissue composition and density. Areas of high cellularity, hemorrhage, or edema can be identified, contributing to the assessment of tumor density. Positron Emission Tomography (PET) imaging, combined with radiolabeled tracers, can assess tumor density by measuring metabolic activity. PET scans detect the distribution and concentration of radiotracers, which are taken up by metabolically active cells. Areas of high metabolic activity can indicate regions of increased cellularity and higher tumor density. PET scans often provide functional and metabolic information in conjunction with anatomical imaging modalities. Histopathological examination of tumor tissue sections is crucial for assessing tumor density at the cellular level. Hematoxylin and eosin (H&E) staining allows for the visualization of tumor architecture and cellularity. Pathologists examine the tissue sections under a microscope to identify areas of high cell density or stromal components, providing insights into tumor density and composition. Image analysis software can be used to quantitatively analyze tumor density based on imaging data. These tools can segment the tumor region and calculate parameters such as the fraction of high-density regions, low-density regions, or overall tumor density. Automated image analysis allows for standardized and objective assessments of tumor density. Radiomics, the extraction and analysis of quantitative features from medical images, can also provide information about tumor density. Texture analysis, a subset of radiomics, focuses on quantifying spatial variations in pixel intensities. Texture analysis can provide insights into tumor heterogeneity and density patterns, helping to characterize tumor density and its changes over time.

In some embodiments, the methods comprise assaying for decreased tumor markers. Some types of cancer produce specific biomarkers that can be measured in the blood, and a reduction in these markers after treatment can indicate a positive response. Assaying for decreased tumor markers is an important aspect of cancer diagnosis, monitoring treatment response, and assessing disease progression. Tumor markers are substances produced by tumor cells or the body in response to cancer. Elevated levels of specific tumor markers in blood or other biological samples can indicate the presence of cancer. Monitoring changes in tumor marker levels, particularly a decrease in their concentration, can provide valuable insights into treatment effectiveness and disease status. Blood tests are commonly used to measure tumor marker levels. Techniques such as ELISA or radioimmunoassay (RIA) allow for the quantification of specific tumor markers in the bloodstream. A decrease in tumor marker concentration over time can indicate a positive response to treatment. These blood tests provide a non-invasive and relatively accessible method for assessing tumor marker levels. In addition to individual tumor markers, panels of multiple biomarkers may be assessed to provide a comprehensive assessment of cancer status. These panels can include various tumor markers that are associated with a specific type of cancer or provide complementary information. By monitoring the levels of multiple tumor markers within a panel, a decrease in their concentrations can provide a more robust assessment of treatment response. Imaging modalities, such as CT, MRI, or PET, can be used to visualize tumors and assess changes in tumor size or metabolic activity. A decrease in tumor size or metabolic activity observed through imaging can be correlated with a decrease in tumor marker levels. Combining imaging techniques with tumor marker assays offers a multi-dimensional assessment of treatment response and disease progression. Tumor marker levels can also be assessed through biopsy and histopathological analysis of tumor tissue. Tissue samples obtained through biopsy can be analyzed using techniques such as immunohistochemistry or in situ hybridization to detect and quantify tumor marker expression. A decrease in tumor marker staining intensity or extent can indicate treatment response at the tissue level. Molecular testing techniques, such as polymerase chain reaction (PCR) or next-generation sequencing (NGS), can be employed to assess tumor marker expression at the genetic or molecular level. These techniques allow for the quantification of specific genetic alterations or gene expression changes associated with tumor markers. A decrease in the expression or presence of these markers can indicate treatment response at the molecular level.

In some embodiments, the methods comprise assaying for slowed tumor growth. In some cases, cancer treatments may not shrink the tumor but can slow its growth, which can still be considered a positive response to treatment. Assaying for slowed tumor growth is a key component of assessing treatment efficacy and monitoring the progression of cancer. Various methods are employed to assay for slowed tumor growth, providing valuable insights into treatment effectiveness and disease management. Imaging techniques, such as CT, MRI, or PET, play a crucial role in visualizing tumors and assessing changes in their size over time. By comparing tumor measurements taken at different time points, the rate of tumor growth can be evaluated. Imaging also allows for the identification of new lesions or the absence of tumor progression, indicating successful treatment and slowed tumor growth. Tumor volume measurements provide quantitative assessments of tumor size using manual or automated methods. This can be done through caliper measurements, 3D reconstruction of imaging data, or volumetric analysis. Tracking changes in tumor volume over time enables the evaluation of tumor growth rate and identification of any deceleration or stabilization, indicating slowed tumor growth. Monitoring specific biomarkers associated with tumor growth can provide additional insights into treatment response. Tumor markers, such as PSA or carbohydrate antigen 125 (CA-125), can be measured in blood samples. A decrease or stabilization in the levels of these markers suggests a reduced rate of tumor growth, indicating a positive response to therapy. Histopathological analysis of tumor tissue obtained through biopsies or surgical resections allows for direct examination of tumor characteristics. Pathologists assess the size and cellular features of tumor cells under a microscope. Decreased cellular proliferation, reduced mitotic activity, or a decrease in tumor grade can indicate a slowing of tumor growth, providing evidence of treatment effectiveness. Immunohistochemical staining for proliferation markers, such as Ki-67, provides insights into the rate of cell division within tumors. A decrease in the proportion of proliferating cells indicates slowed tumor growth. This approach allows for the assessment of treatment response at the cellular level and provides valuable information about the biological activity of the tumor. Molecular profiling techniques, such as gene expression analysis or genomic sequencing, offer insights into alterations in gene expression patterns associated with tumor growth. Decreased expression of genes associated with cell proliferation or tumor progression suggests a reduction in tumor growth rate, indicating a positive response to treatment.

In some embodiments, the methods comprise assaying for elimination of metastases. Some treatments, such as radiation therapy or targeted therapies, may be able to eliminate cancer that has spread to other parts of the body. Assaying for the elimination of metastases is a crucial aspect of cancer management and monitoring the effectiveness of treatments. Metastases, which refer to the spread of cancer cells from the primary tumor to distant sites in the body, pose significant challenges in cancer treatment. Detecting and assessing the elimination of metastases are essential for evaluating treatment response and determining disease progression. Imaging techniques, such as CT, MRI, or PET, play a key role in visualizing metastatic lesions in various organs. Regular imaging scans allow for the assessment of the size, number, and location of metastatic lesions. The elimination of metastases is indicated by the disappearance or significant reduction in the size and number of lesions over time, providing evidence of successful treatment response. Monitoring specific tumor markers associated with metastatic disease is another valuable approach. Tumor markers, such as CEA or PSA, can be measured in blood samples. A decrease or normalization of tumor marker levels indicates the elimination or effective control of metastatic disease. Tracking changes in tumor marker concentrations provides insights into treatment response and the elimination of metastatic lesions. Biopsy or surgical removal of metastatic lesions allows for direct examination of the tissue to confirm the absence of cancer cells. Histopathological analysis of the biopsy samples helps determine if the metastatic lesions have been eliminated. The absence of malignant cells in the biopsy specimen indicates successful treatment and elimination of metastases, providing definitive evidence of treatment efficacy. Molecular testing techniques, such as circulating tumor DNA (ctDNA) analysis or next-generation sequencing (NGS), can be employed to detect the presence or absence of specific genetic alterations associated with metastatic disease. The absence of genetic mutations or alterations previously detected in metastatic lesions indicates the elimination of those metastatic clones, further supporting the notion of treatment success.

In some embodiments, the methods comprise assaying for increased survival. In some embodiments, the methods comprise assaying for improved symptoms. If cancer is causing symptoms, such as pain or difficulty breathing, successful treatment can lead to an improvement in these symptoms. In some embodiments, the methods comprise assaying for improved overall health. Cancer treatments can improve overall health, such as improving blood counts or reducing inflammation, even if the tumor size remains stable.

### Additional Aspects

Additional aspects of the disclosure are provided by the following numbered paragraphs:

1. A method comprising:
administering cells from a T cell-negative fraction to an immunodeficient mouse, wherein the immunodeficient mouse has been engrafted with diseased human cells.

2. A method comprising:
administering diseased human cells to an immunodeficient mouse; and administering cells from a T cell-negative fraction to the immunodeficient mouse.

3. The method of paragraph 1 or 2, wherein the cells are human peripheral blood mononuclear cells (PBMCs), optionally wherein the human PBMCs are isolated from the T cell-negative fraction.

4. The method of any one of paragraphs 1-3, further comprising administering to the immunodeficient mouse a therapeutic agent, optionally about 3 to about 10 days, preferably about 6 days, after administering the cells of the T cell-negative fraction.

5. The method of paragraph 4, wherein the therapeutic agent is selected from an engineered immune cell, a recombinant protein, a nucleic acid, and a small molecule drug.

6. The method of paragraph 5, wherein the therapeutic agent is an engineered immune cell that comprises a receptor that specifically binds to a cell surface antigen on the diseased human cells.

7. The method of any one of paragraphs 6, wherein the receptor is a chimeric antigen receptor (CAR) or a T cell receptor.

8. The method of paragraph 7, wherein the engineered cell is a T cell, an NK cell, or a B cell.

9. The method of paragraph 8, wherein the T cell is a regulatory T cell (Treg) or a tumor infiltrating lymphocyte (TIL).

10. The method of paragraph 5, wherein the recombinant protein is an antibody, optionally an antibody fragment.

11. The method of paragraph 10, wherein the nucleic acid is an antisense oligonucleotide (ASO), a short interfering RNA (siRNA), a messenger RNA (mRNA), or a viral vector, optionally an adeno-viral vector (AAV).

12. The method of any one of paragraphs 4-11, further comprising assaying the immunodeficient mouse for symptoms of cytokine release syndrome (CRS) and/or efficacy of the therapeutic agent.

13. The method of any one of the preceding paragraphs, wherein the diseased human cells are selected from blood cells, muscle cells, and neuronal cells.

14. The method of any one of the preceding paragraphs, wherein the diseased human cells are tumor cells.

15. The method of paragraph 14, wherein the tumor cells are primary tumor cells.

16. The method of any one of paragraphs 1-15, wherein the diseased human cells are non-cancerous cells.

17. The method of any one of paragraphs 1-15, wherein the diseased human cells are cancerous cells.

18. The method of paragraph 17, wherein the cancerous cells are pediatric cancerous cells.

19. The method of paragraph 1 or 18, wherein the cancerous cells are from a subject having Stage 3 or Stage 4 cancer.

20. The method of paragraph 19, wherein the T cell-negative fraction is obtained from a subject having Stage 3 or 4 cancer.

21. The method of paragraph 19 or 20, wherein the subject has undergone a first-line cancer therapy and/or a second-line cancer therapy.

22. The method of any one of the preceding paragraphs, wherein the diseased human cells and the human PBMCs or human T cell-negative fraction cells are autologous relative to each other.

23. The method of any one of paragraphs 1-22, wherein the diseased human cells and the human PBMCs or human T cell-negative fraction cells are allogeneic relative to each other.

24. The method of any one of the preceding paragraphs, further comprising administering a myeloablative treatment to the immunodeficient mouse, prior to administering the human PBMCs or the human T cell-negative fraction cells to the immunodeficient mouse, optionally wherein the myeloablative treatment comprises a myeloablative chemical treatment or sublethal irradiation.

25. The method of any one of paragraphs 3-19, comprising administering to the immunodeficient mouse simultaneously: (1) the therapeutic agent; and (2) the human PBMCs or human T cell-negative fraction cells.

26. The method of any one of paragraphs 12-25, further comprising administering to the immunodeficient mouse a candidate agent for treating CRS prior to the assaying.

27. The method of any one of the preceding paragraphs, wherein the mouse has a non-obese diabetic (NOD) genetic background.

28. The method of any one of the preceding paragraphs, wherein the mouse comprises a null mutation in a Prkdc gene, optionally a Prkdc^{scid} allele, and a null mutation in an Il2rg gene, optionally a IL2rg^{tmlwJl} allele.

29. The method of paragraph 28, wherein the mouse has a NOD-Cg.-Prkdc^{scid} IL2rg^{tmlwJl}/SzJ genetic background.

30. The method of any one of the preceding paragraphs, wherein the mouse lacks functional major histocompatibility complex I (MHC I) and major histocompatibility complex II (MHC II).

31. The method of any one of the preceding paragraphs, wherein mouse comprises a null H2-Abl gene, optionally a H2-Abl^{emlMvw} allele, a null MHC Class I H2-Kl gene, optionally a H2-Kl^{tmlBpe} allele, and/or a null MHC Class I H2-Dl gene, optionally a H2-Dl^{tmlBpe} allele.

32. The method of any one of paragraphs 27-31, wherein the mouse has a NOD.Cg-Prkdc^{scid} H2-Kl^{tmlBpe} H2-Abl^{emlMvw} H2-Dl^{tmlBpe} Il2rg^{tmlWjl}/SzJ mouse (NSG-(Kb Db)^{null} (LA^{null}) mouse) genetic background.

33. The method of any one of paragraphs 1-32, wherein the immunodeficient mouse comprises a transgene encoding human interleukin-3 (IL-3), a transgene encoding human granulocyte/macrophage-colony stimulating factor 2 (GM-CSF), a transgene encoding human stem cell factor (SCF), and optionally further comprises a transgene encoding human macrophage colony-stimulating factor 1 (CSF1) and/or a transgene encoding human IL-15.

34. The method of paragraph 33, wherein the immunodeficient mouse has a NOD-scid IL2Rgammanull genetic background.

35. The method of paragraph 34, wherein the mouse has a NOD.Cg-Prkdc^{scid} Il2rg^{tmlWjl} Tg (CMV-IL3,CSF2,KITLG) 1Eav Tg (IL15) 1Sz/J genetic background.

36. The method of any one of paragraphs 1-35, wherein the immunodeficient mouse comprises human FLT3L protein and/or a null mutation in a mouse Flt3 gene.

37. The method of paragraph 36, wherein the immunodeficient mouse has a NOD.Cg-Flt3^{em2Mvw} Prkdc^{scid} Il2rg^{tmlWjl} Tg (FLT3LG) 7Sz/SzJ genetic background.

38. The method of any one of the preceding paragraphs, wherein the administering is intravenous, optionally by tail vein injection, or intraperitoneal.

39. The method of any one of the preceding paragraphs, wherein about 1×10\hat{ }6 to 1×10\hat{ }8, optionally about 1.5×10\hat{ }7, cells from the T cell-negative fraction are administered.

40. The method of any one of the preceding paragraphs, wherein the T cell-negative fraction is a CD3-T cell-negative fraction, optionally a CD3-CD4-T cell-negative fraction and/or CD3-CD8-T cell-negative fraction.

41. The method of any one of paragraphs 12-40, wherein the assaying comprises measuring a circulating level of a cytokine selected from the group consisting of: interleukin (IL)-6, IL10, interferon (IFN)-γ, monocyte chemoattractant protein 1 (MCP-1), granulocyte-macrophage colony-stimulating factor (GM-CSF), tumor necrosis factor (TNF), IL-1, IL-2, IL-2-receptor alpha (IL-2Rα), IL-8, IL-4, IL-18, and macrophage inflammatory protein (MIP) 4, preferably IL-6, IL-10, and/or IFN-γ.

### EXAMPLES

### Example 1—Effects of T Cell-negative Fraction on Different Strains of Mice

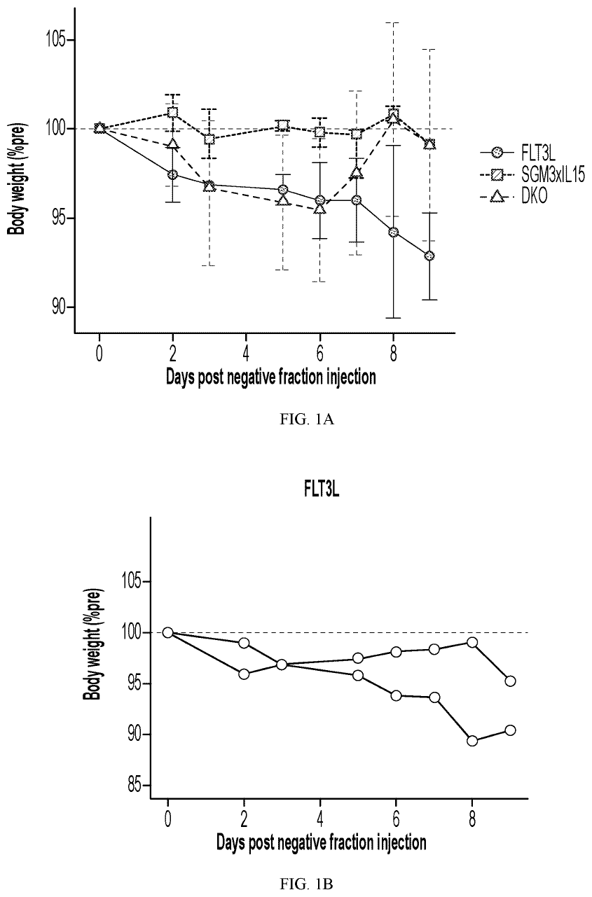

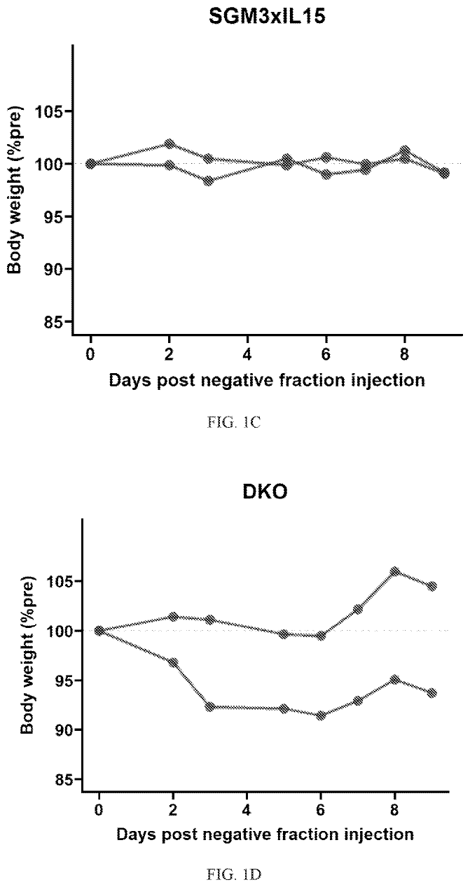

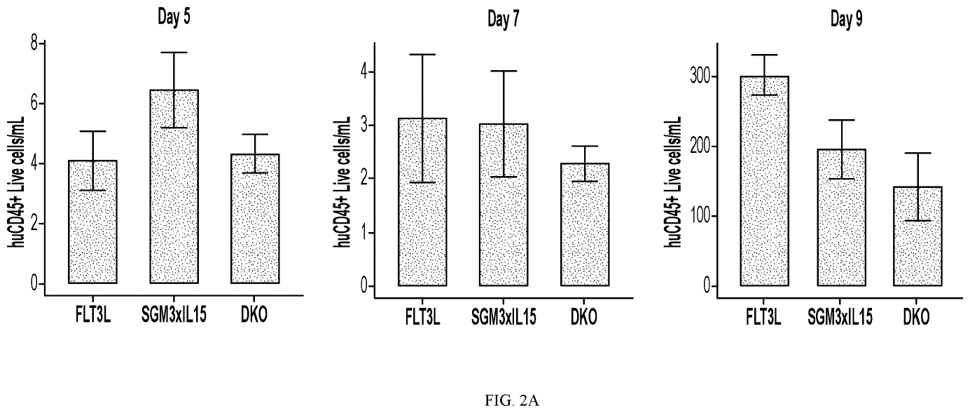

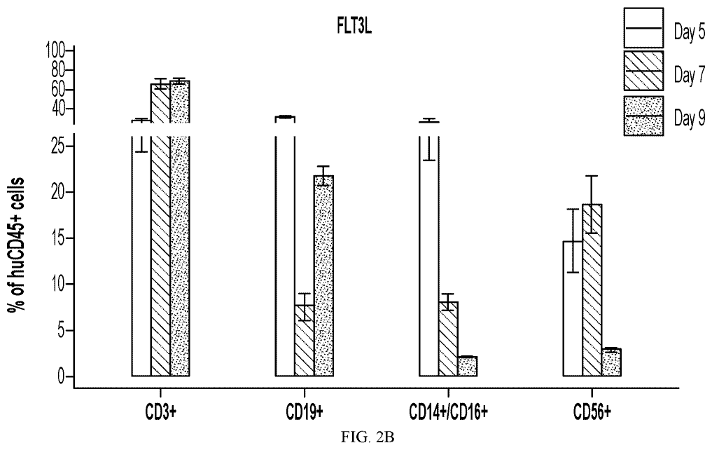

The effects of T cell-negative fraction on three different mouse strains (NSG™-MHC class I/II DKO (“DKO”) (NOD.Cg-Prkdc^{scid} H2-Kl^{tmlBpe} H2-Abl^{emlMvw} H2-Dl^{tmlBpe} Il2rg^{tmlWjl}/SzJ; The Jackson Laboratory Strain #: 025216), SGM3xIL15 (NOD.Cg-Prkdc^{scid} Il2rg^{tmlWjl} Tg (CMV-IL3,CSF2,KITLG) 1Eav Tg (IL15) 1Sz/J; The Jackson Laboratory Strain #: 033216), and FLT3L (NOD.Cg-Flt3^{em2Mvw} Prkdc^{scid} Il2rg^{tmlWjl} Tg (FLT3LG) 7Sz/SzJ; The Jackson Laboratory Strain #: 033367)) were examined. Briefly, mice (n=2/group) were engrafted with human patient CD3-negative fraction (1.5×10⁷cells/mouse). Body weight was measured over time in different strains. As can be seen in FIGS. 1A-1D, both SMG3xIL 15 mice maintained their body weight throughout the time course (FIGS. 1A and 1C), while the body weights fell following injection of the negative fraction in the DKO (FIG. 1D) and FLT3L (FIG. 1B) mice.

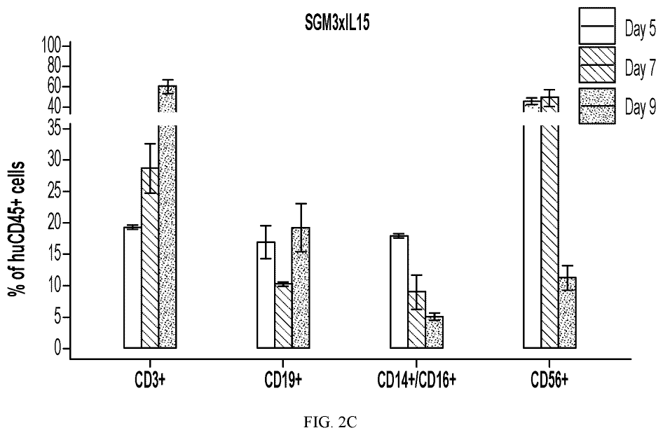

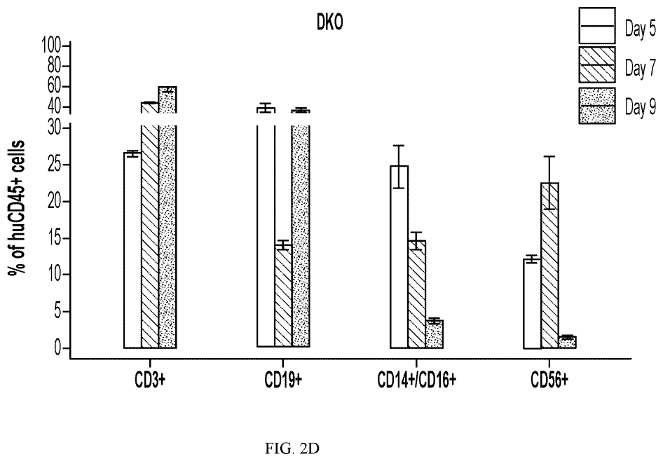

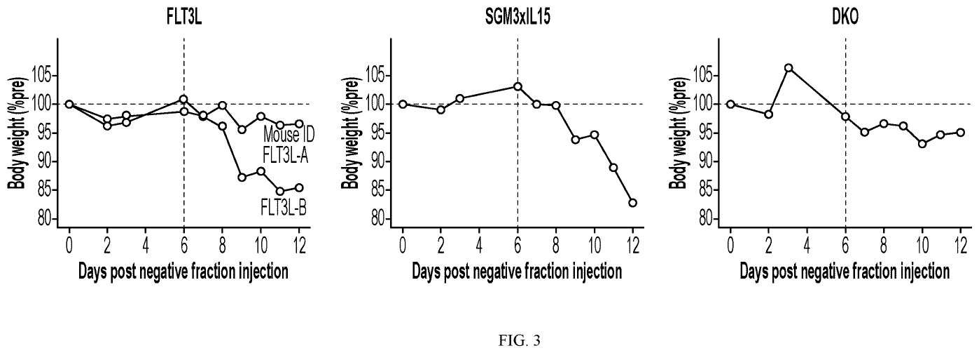

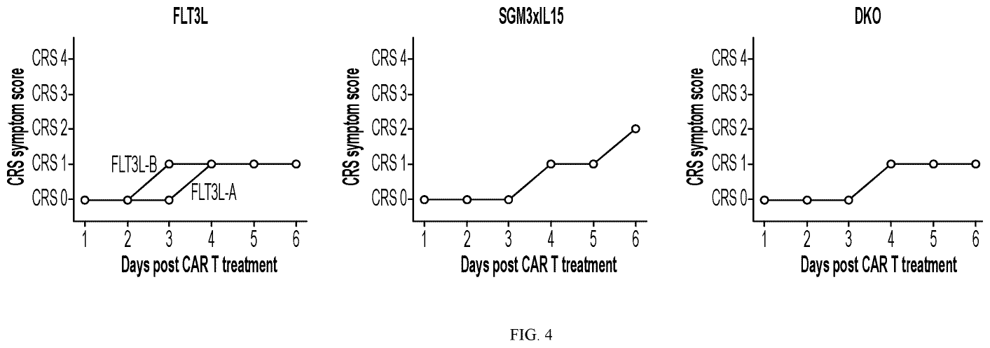

Blood samples were taken from the mice 5, 7, and 9 days after engraftment with human patient CD3-negative fraction (1.5×10⁷cells/mouse). Using flow cytometry, the samples were analyzed for immune cell subsets (CD3+, CD19+, CD14+/16+, CD56+) and total human CD45+ cell counts per microliter of blood. As shown in FIG. 2A, the FLT3L mice had the highest concentration of live human CD45+ cells (as a marker of humanization) at day 9, while the SGM3xIL15 mice had the highest concentration of live human CD45+ cells at day 5 post-engraftment. With respect to the immune cell subsets, the CD45+ cells were found to be mainly CD3+ or CD19+ cells in FLT3L mice (FIG. 2B), SGM3xIL15 mice (FIG. 2C), and DKO mice (FIG. 2D).

### Example 2

### Effects of CAR T Treatment on Different Strains of Mice

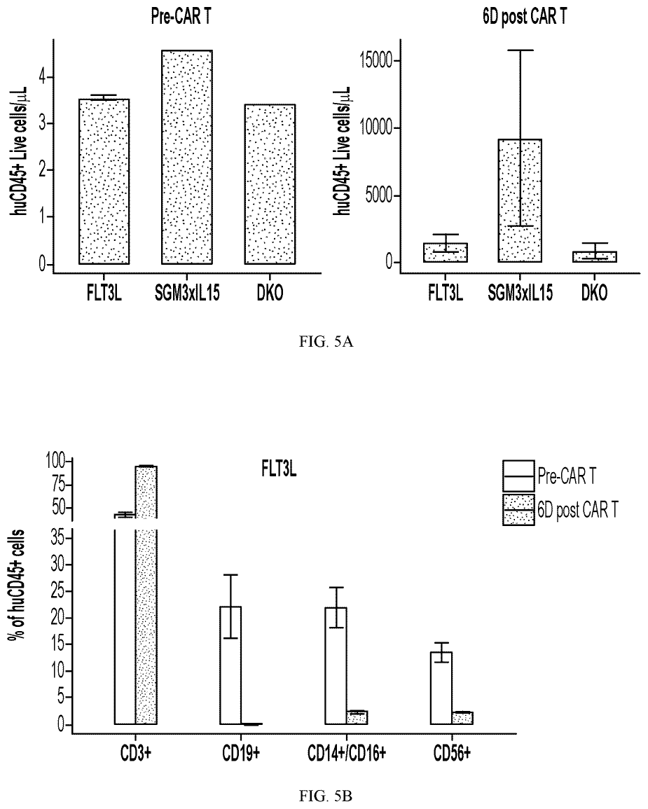

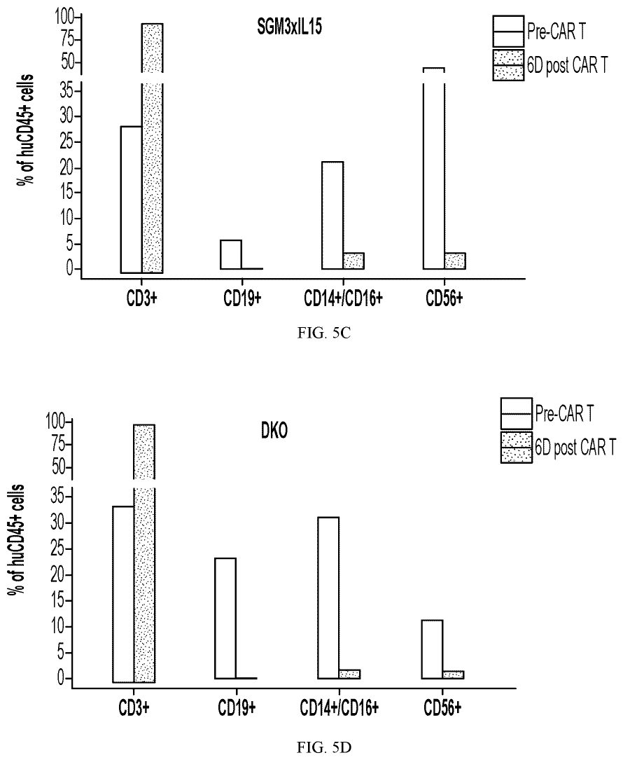

The effects of CAR T treatment following humanization with the T cell-negative fraction were examined. Two FLT3L mice, one SGM3xIL 15 mouse, and one DKO mouse were each engrafted with human negative fraction cells (1.5×10⁷ cells/mouse). Six days after engraftment, each mouse was treated with allogeneic CAR T cells (5×10⁶ cells/mouse). Body weight was measured over time, and one FLT3L mouse and one SGM3xIL15 mouse showed significant body weight loss 3 days after the CAR T treatment (FIG. 3). The mice were also given clinical scores for cytokine release syndrome (CRS) symptoms following CAR T treatment (FIG. 4).

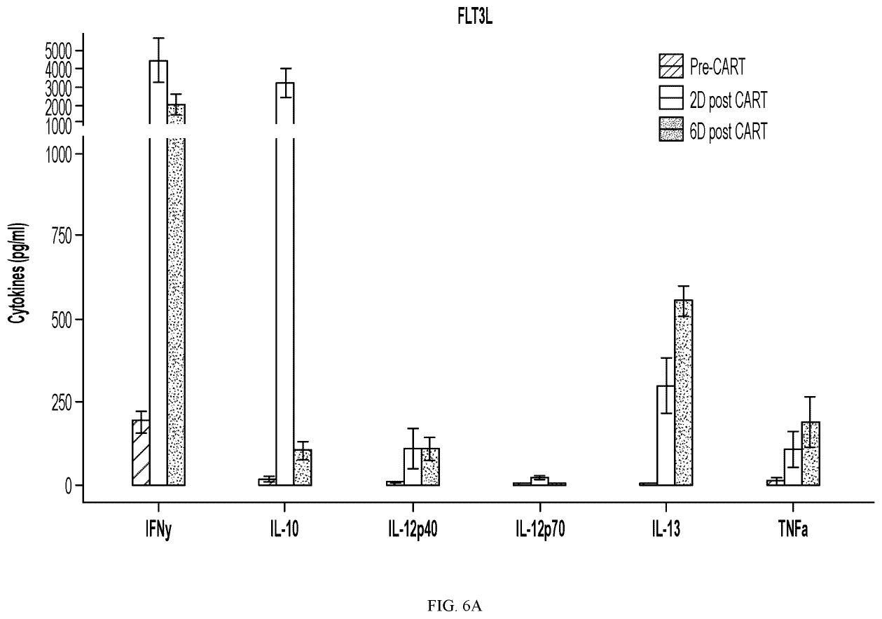

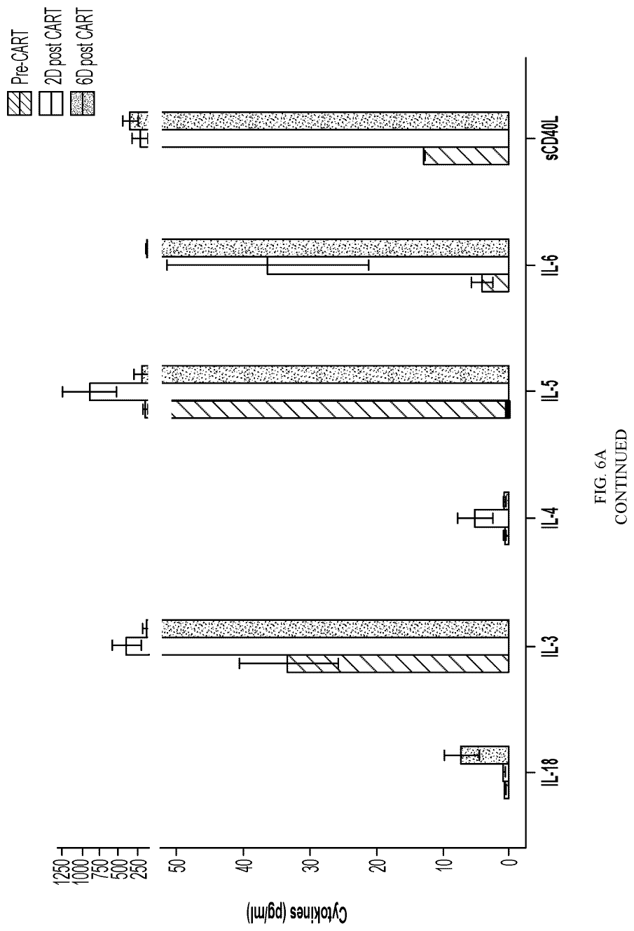

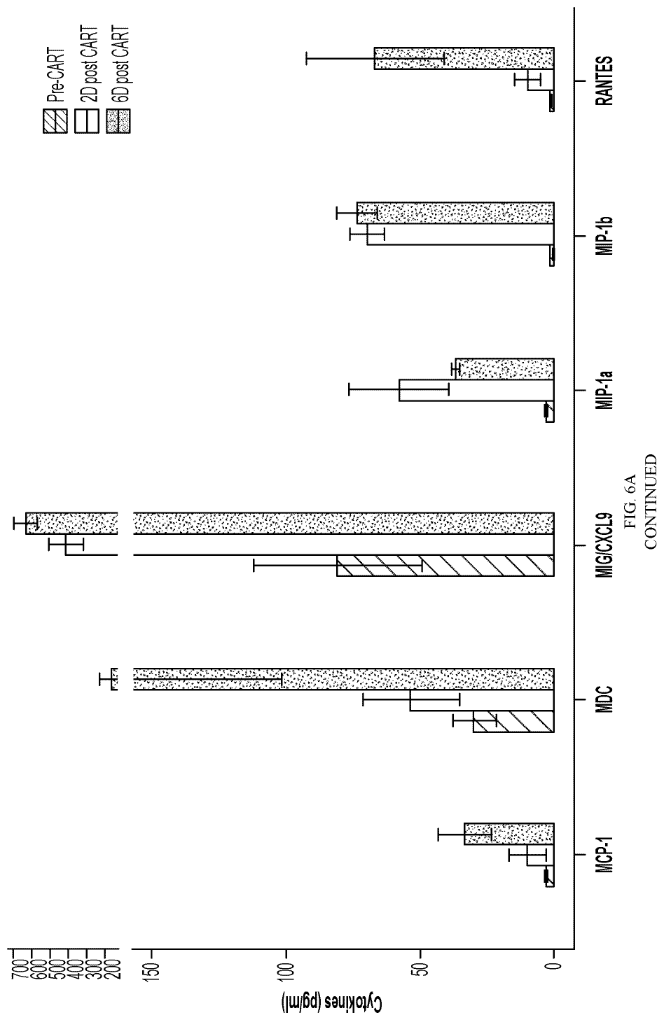

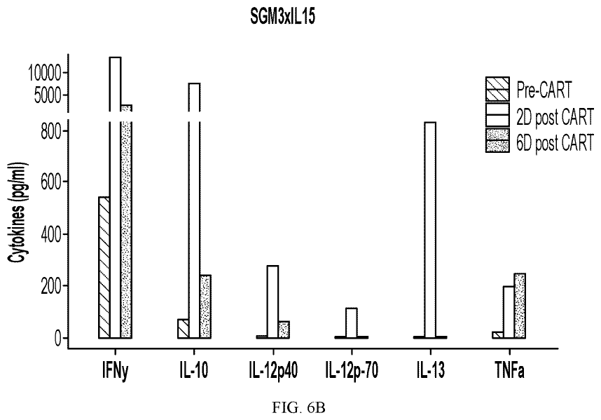

In addition, blood samples were collected on the day of treatment (4 hour prior to the treatment) and 6 days post-treatment and analyzed for immune cell subsets (CD3+, CD19+, CD14+/16+, CD56+) and total human CD45+cell counts per microliter of blood by flow cytometry. The CAR T treatment was found to be efficacious, as shown from the B cell population (CD19+), which is almost 0% 6 days post-CAR T treatment (FIGS. 5B-5D). Correspondingly, the percentage of T cells (CD3+) increased significantly as a function of CAR T cell expansion, particularly in the SGM3xIL15 mouse (FIG. 5C).

Two FLT3L mice, one SGM3xIL15 mouse, and one DKO mouse were each engrafted with human negative fraction cells (1.5×10⁷ cells/mouse). Six days after engraftment, each mouse was intravenously injected with CAR T cells (5×10⁶ cells/mouse). Blood samples were taken four hours prior to treatment (Pre-CAR T), two days post-treatment (2D post-CAR T), and six days post-treatment (6D post-CAR T), and circulating human cytokine concentrations were measured by Milliplex Human Cytokine/Chemokine/Growth Factor

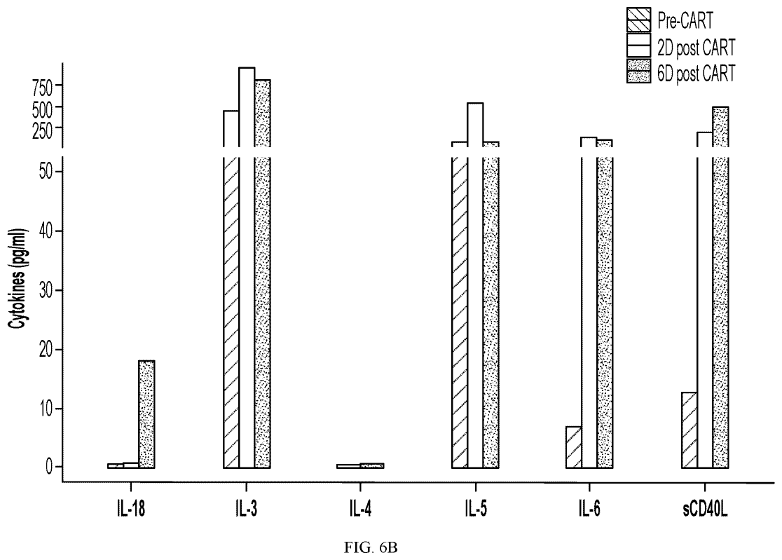

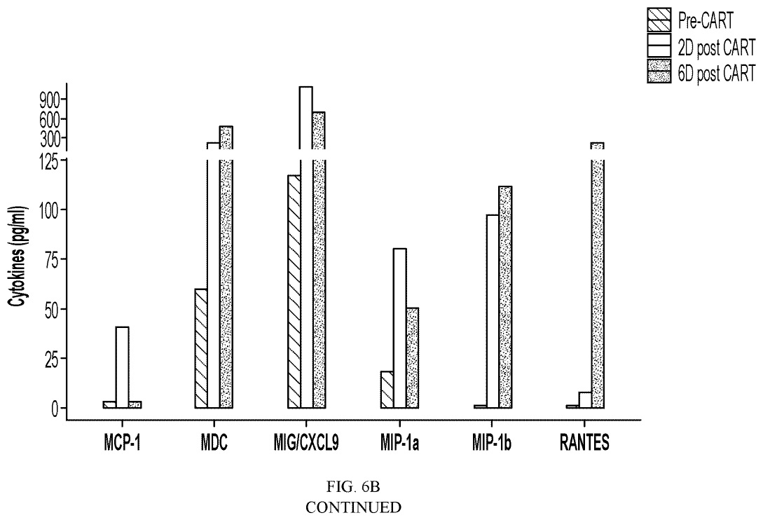

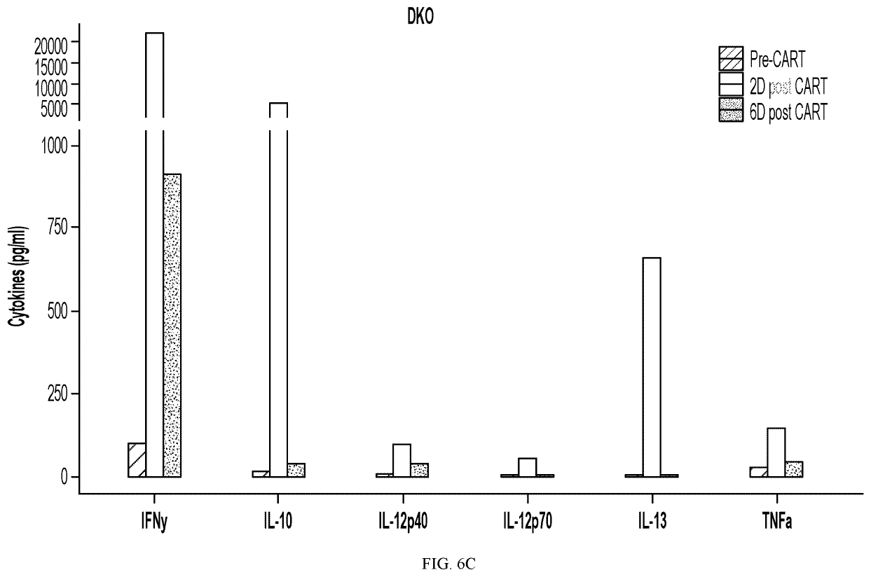

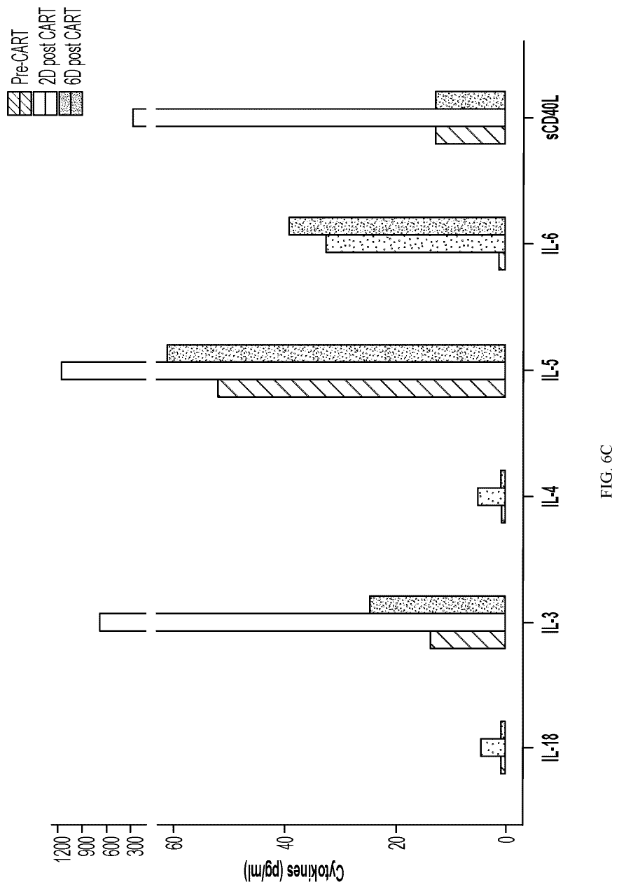

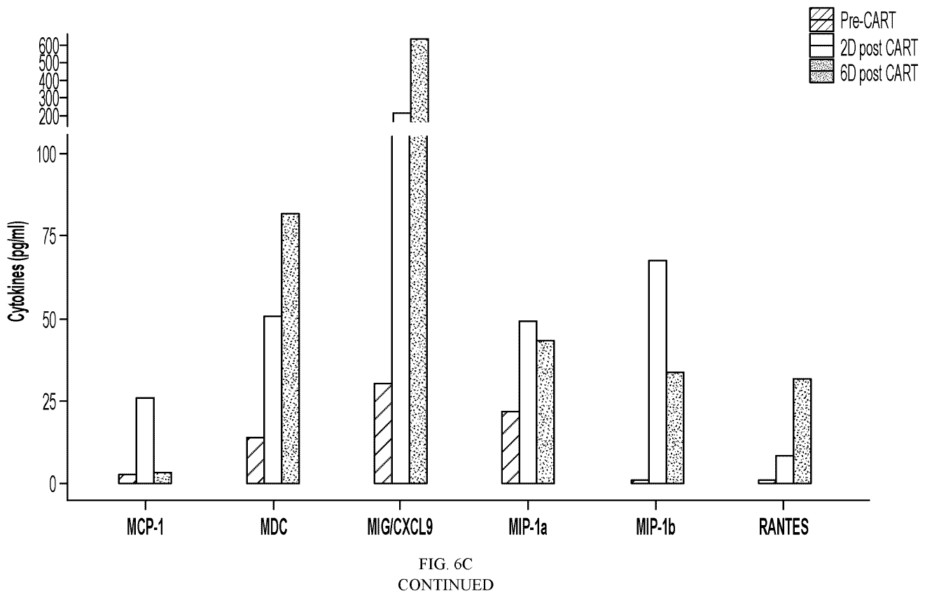

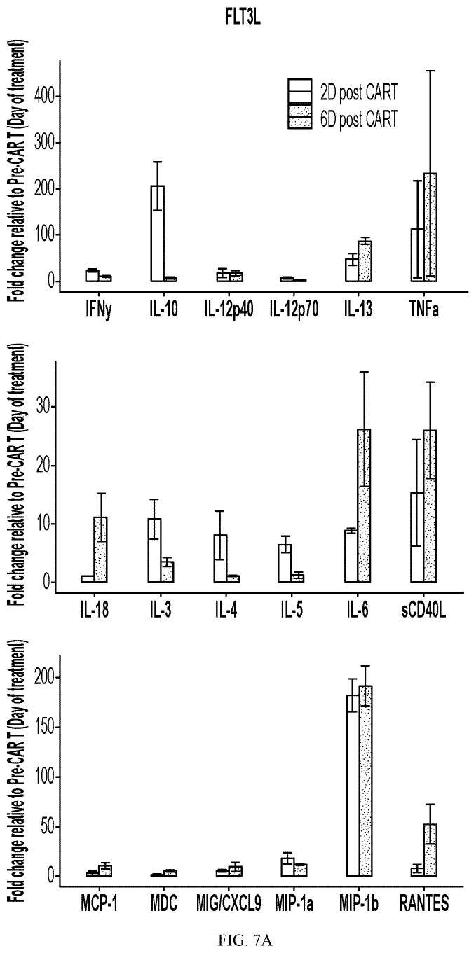

Panel A 48 Plex kit. The results are shown in FIGS. 6A-6C. The fold-change of cytokine levels measured two and six days after the CAR T treatment compared to the baseline level are shown in FIGS. 7A-7C.

### Example 3—Effects of Raw Negative Fraction and Processed PBMC Negative Fraction

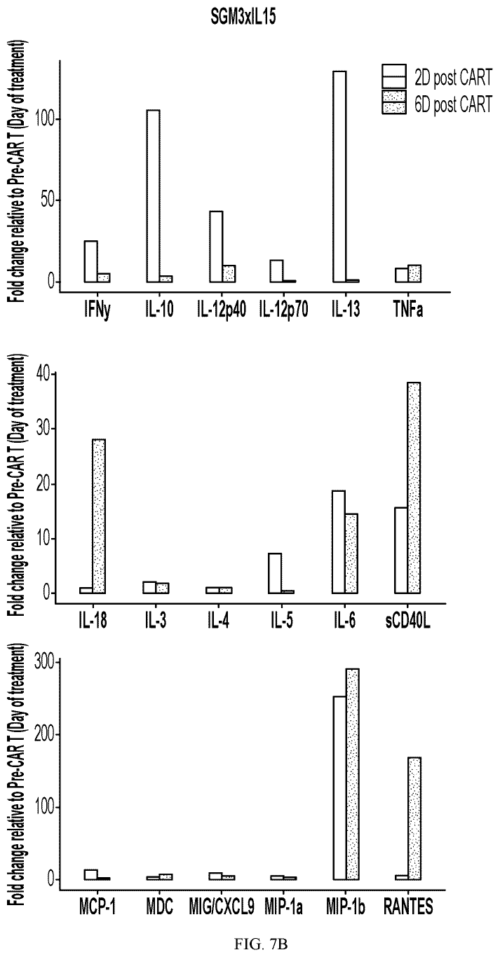

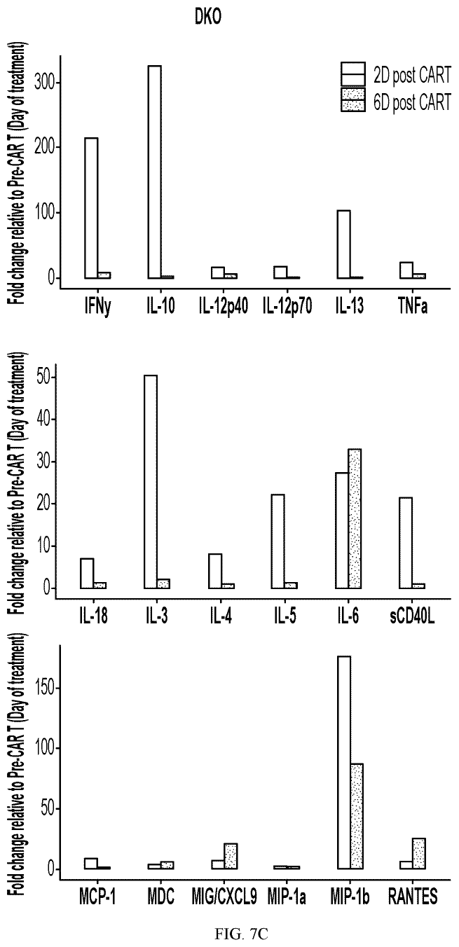

The effects of humanizing SGM3xIL15 mice with raw negative fractions (“raw”) and negative fractions processed to only include PBMCs (“PBMC”) were examined. With respect to body weight, the injection of 1.5×10⁷ raw negative fraction cells or processed PBMCs did not have a different effect over time (FIG. 8A). When mice were intravenously injected with allogeneic CAR T cells (5×10⁶ cells/mouse) six days after engraftment, the results were similar (FIG. 8B).

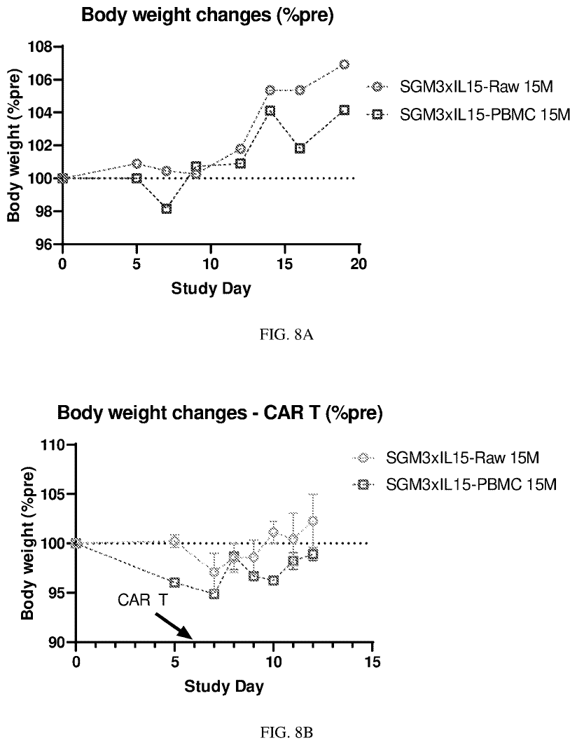

The number of live cells were examined on day 6 (prior to any CAR T treatment), and the results are shown in FIG. 9. The SGM3xIL15 mice engrafted with 1.5×10⁷ processed PBMCs had higher human immune cell engraftment than the mice engrafted with a 1.5×10⁷ raw negative fraction. In addition, the hCD19 cells (a target population for CAR T therapies) were notably high in the processed PBMC group.

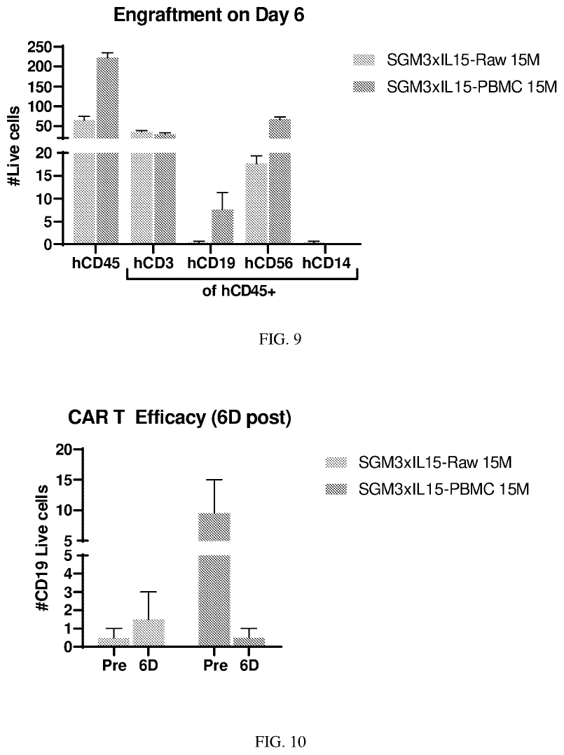

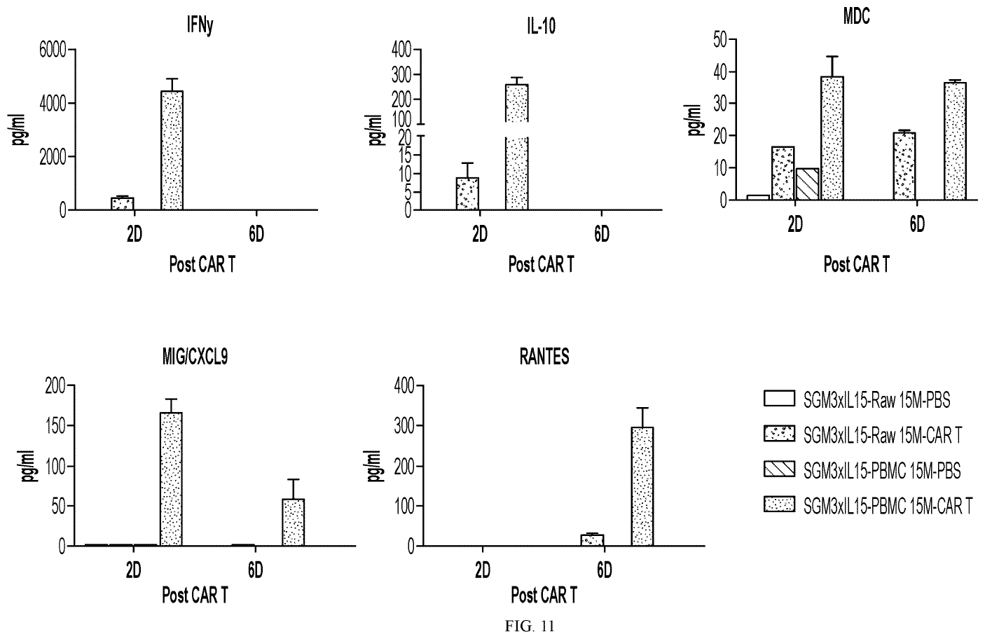

Next, the effects of allogenic CAR T therapy were examined. Allogeneic CAR T treatment showed great efficacy only in processed PBMC group, six days post-treatment (FIG. 10). Cytokine levels were also examined two and six days after allogeneic CAR T therapy. CAR T treatment induced human cytokine release in mice humanized with both raw negative fraction and processed PBMCs; however, the cytokine levels were much higher with processed PBMCs, and some cytokines (e.g. MIG/CXCL9) were only induced in mice humanized with processed PBMCs (FIG. 11).

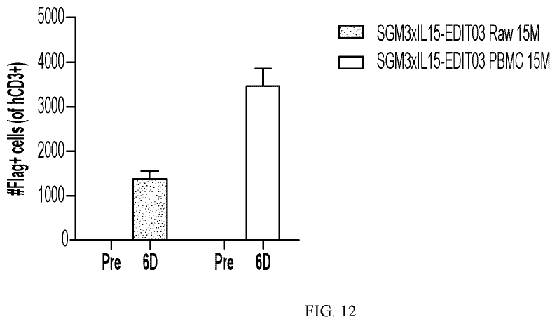

Allogeneic CAR T therapy was also investigated, and T cell expansion was measured before CAR T therapy and six days post-autologous CAR T therapy. Allogeneic CAR T cells expanded well in both groups at the post-treatment time point; however, the degree of expansion was greater in mice humanized with processed PBMCs (FIG. 12).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein. What is claimed is:

1. A method comprising:
administering a T cell-negative fraction, or cells from the T cell-negative fraction, to a mouse, optionally an immunodeficient mouse, wherein the T cell-negative fraction is from a cancer patient.

2. The method of claim 1 further comprising administering human cells to the mouse, optionally wherein the human cancer cells are from the cancer patient.

3. The method of claim 1 or 2 further comprising administering a therapeutic agent to the mouse.

4. The method of any one of the preceding claims further comprising assaying the mouse for one or more human cytokines prior to the onset of graft-versus-host disease in the mouse.

5. The method of any one of the preceding claims, wherein about 1×10\hat{ }6 to about 1×10\hat{ }8 cells, optionally about 0.5×10\hat{ }7 to about 3×10\hat{ }7 cells, from the T cell-negative fraction are administered to the mouse.

6. The method of any one of the preceding claims, wherein the T cell-negative fraction comprises about 1×10\hat{ }6 to about 1×10\hat{ }8 cells, optionally about 0.5×10\hat{ }7 to about 3×10\hat{ }7 cells.

7. The method of any one of the preceding claims, wherein the cells are human peripheral blood mononuclear cells (PBMCs).

8. The method of any one of the preceding claims, wherein the cancer patient is a Stage 3 or Stage 4 cancer patient, optionally wherein the cancer patient has undergone one or more anti-cancer therapies.

9. The method of any one of the preceding claims, wherein the cancer patient is younger than 18 years old.

10. The method of any one of the preceding claims, wherein the therapeutic agent is administered within 10 days of administering the T cell-negative fraction, or the cells from the T cell-negative fraction, to the mouse.

11. The method of any one of the preceding claims, wherein the therapeutic agent is selected from an engineered immune cell, a recombinant protein, a nucleic acid, and a small molecule drug.

12. The method of claim 11, wherein the therapeutic agent is an engineered immune cell.

13. The method of claim 12, wherein the engineered immune cell is a T cell, an NK cell, or a B cell.

14. The method of claim 13, wherein the engineered immune cell is a T cell, optionally a regulatory T cell (Treg) or a tumor infiltrating lymphocyte (TIL).

15. The method of any one of claims 12-14, wherein the engineered immune cell comprises a chimeric antigen receptor (CAR) or a T cell receptor.

16. The method of claim 11, wherein the recombinant protein is an antibody, optionally an antibody fragment.

17. The method of claim 11, wherein the nucleic acid is an antisense oligonucleotide (ASO), a short interfering RNA (siRNA), a messenger RNA (mRNA), or a viral vector, optionally an adeno-viral vector (AAV).

18. The method of any one of the preceding claims, wherein the assaying is within 10 days of administering the therapeutic agent to the mouse.

19. The method of any one of the preceding claims, wherein the one or more human cytokines is selected from interleukin-6 (IL-6), IL-10, and interferon (IFN)-γ.

20. The method of any one of the preceding claims, wherein the mouse has undergone a myeloablative treatment, optionally gamma irradiation.

21. The method of any one of the preceding claims, wherein the mouse is an immunodeficient mouse.

22. The method of any one of the preceding claims, wherein the mouse has a non-obese diabetic (NOD) genetic background.

23. The method of any one of the preceding claims, wherein the mouse comprises a null mutation in a Prkdc gene, optionally a Prkdc^{scid} allele, and a null mutation in an Il2rg gene, optionally a IL2rg^{tmlwJl} allele.

24. The method of any one of the preceding claims, wherein the mouse comprises a null H2-Abl gene, optionally a H2-Abl^{emlMvw} allele, a null MHC Class I H2-Kl gene, optionally a H2-Kl^{tmlBpe} allele, and/or a null MHC Class I H2-Dl gene, optionally a H2-Dl^{tmlBpe} allele.

25. The method of any one of the preceding claims, wherein the mouse comprises a transgene encoding human interleukin-3 (IL-3), a transgene encoding human granulocyte/macrophage-colony stimulating factor 2 (GM-CSF), a transgene encoding human stem cell factor (SCF), and optionally further comprises a transgene encoding human macrophage colony-stimulating factor 1 (CSF1) and/or a transgene encoding human IL-15.

26. The method of any one of the preceding claims, wherein the mouse comprises a nucleic acid encoding human FLT3L protein and/or a null mutation in a mouse Flt3 gene, optionally a Flt3^{em2Mvw} allele.

27. The method of any one of the preceding claims, wherein the administering is intravenous, optionally by tail vein injection, or intraperitoneal.

28. The method of any one of the preceding claims, wherein the T cell-negative fraction is a CD3⁻T cell-negative fraction, optionally a CD3⁻CD4⁻T cell-negative fraction and/or CD3⁻CD8⁻T cell-negative fraction.
